(19) United States
(12) Patent Application Publication
(10) Pub. No.: US 2024/0024301 A1
(43) Pub. Date: Jan. 25, 2024

(54) METHODS OF TREATING HEAD AND NECK SQUAMOUS CELL CARCINOMA

(71) Applicant: Kura Oncology, Inc., San Diego, CA (US)

(72) Inventors: Francis Burrows, Solana Beach, CA (US); Mollie Leoni, Dover, MA (US); Shivani Malik, San Diego, CA (US); Vishnu S. Mishra, San Diego, CA (US); Alison Smith, San Diego, CA (US); Harris Soifer, San Diego, CA (US)

(21) Appl. No.: 18/355,850

(22) Filed: Jul. 20, 2023

Related U.S. Application Data
(63) Continuation of application No. PCT/US2023/065417, filed on Apr. 6, 2023.
(60) Provisional application No. 63/479,683, filed on Jan. 12, 2023.
(60) Provisional application No. 63/380,908, filed on Oct. 25, 2022.
(60) Provisional application No. 63/329,257, filed on Apr. 8, 2022.

(51) Int. Cl. A61K 31/4439; A61P 35/00; A61K 31/4709

(52) U.S. Cl. CPC A61K 31/4439; A61P 35/00; A61K 31/4709

(57) ABSTRACT

Provided herein are methods of tipifarnib in combination with a PI3K inhibitor, such as alpelisib, for treating, preventing or managing head and neck squamous cell carcinoma (HNSCC).

### 1. CROSS REFERENCE

This application claims the benefit of priority from U.S. Provisional Application No. 63/479,683, filed Jan. 12, 2023, U.S. Provisional Application No. 63/380,908, filed Oct. 25, 2022, and U.S. Provisional Application No. 63/329,257, filed Apr. 8, 2022. Each of the foregoing related applications, in its entirety, is incorporated herein by reference.

### 2. FIELD

Provided herein are novel methods of using an active farnesyltransferase inhibitor comprising tipifarnib in combination with a phosphatidylinositol-3-kinase (PI3K) inhibitor. More specifically, the methods provided herein include the use of tipifarnib and alpelisib for treating, preventing, or managing head and neck squamous cell carcinoma (HNSCC). Further provided herein are methods of using tipifarnib and alpelisib for mitigating, slowing the progression of, or overcoming cetuximab resistance in HNSCC subjects currently or previously being treated with cetuximab. Pharmaceutical compositions, kits, and related products are also embodied within this disclosure.

### 3. BACKGROUND

Head and neck squamous cell carcinoma (HNSCC) is the sixth most common invasive carcinoma, accounting for nearly 900,000 new diagnoses annually worldwide (Johnson et al., Nat. Rev. Dis. Primers 6:92 (2020)). Exposure to carcinogens (e.g., tobacco, alcohol) and infection with the human papillomavirus (HPV) are described as the two major etiological causes of HNSCC, with the incidence of HPV-positive tumors rapidly on the rise (Chaturvedi et al., J. Clin. Oncol., 26:612-9 (2008)).

HNSCC is a complex group of malignancies with tumors arising from epithelial linings of the oral cavity, pharynx, and larynx, posing several challenges to treating physicians. Most patients are diagnosed with locally advanced disease and treated with strategies integrating surgery, chemotherapy, and radiotherapy. About 50% of these treated patients will experience a recurrence of disease. About 55% of patients with localized disease survive at least five years (American Cancer Society, Survival rates for oral cavity and oropharyngeal cancer (2020)). Regardless of etiology, once the disease has become advanced, or recurrent and/or metastatic (R/M), rates of survival decrease dramatically. Patients having R/M HNSCC have poor prognosis with a median survival of about 12 months despite treatments, with only an estimated 40% of patients surviving at least 5 years (American Cancer Society (2020)).

While surgical excision, chemotherapy, and radiotherapy remain mainstays of treatment, in the R/M setting, immunotherapies and targeted therapies are quickly gaining favor, especially with the approvals of pembrolizumab and nivolumab for the treatment of R/M HNSCC (Chow, N. Engl. J. Med., 382:60-72 (2020)). Importantly, successful implementation of these therapies requires understanding of HNSCC tumor biology, and therefore biopsy and tumor genetic sequencing are increasingly considered standard of care to determine appropriate treatment options.

Numerous genetic mutations are commonly seen in HNSCC. According to the Cancer Genome Atlas (TCGA) Network, some of the most common mutations include TP53 (84%), CDKN2A (58%), CCDN1 (31%), and PIK3CA (34%) (Cancer Genome Atlas Network, Nature, 517:576-82 (2015)). While mutations are important drivers of tumor biology, there are other factors, including overexpression of non-mutated oncogenic signaling proteins and the influence of the tumor microenvironment, that may regulate tumor growth and survival. For instance, The Cancer Genome Atlas (TCGA) shows the overexpression of HRAS in 25% to 30% of HNSCC patients, indicating a potential dependence on HRAS that may be analogous to HRAS as a driver oncogene to a broader population of HNSCCs (‘cBioPortal for Cancer Genomics’ (2020), https://www.cbioportal.org/). Furthermore, mutant HRAS requires PI3K for activity, and is insufficient to be tumorigenic in isolation (Gupta et al., Cell, 129:957-68 (2007)). Similarly, mutant PI3K requires Ras protein in order to drive tumor biology (Zhao and Vogt, Oncogene, 27:5486-96 (2008)). Understanding the interdependencies of key cellular pathways, e.g., the codependency of HRAS and PI3K pathways, may be particularly important in designing combination regimens. Interestingly, HRas protein preferentially activates PI3K five-fold more efficiently than KRas protein, while KRas protein is a more efficient activator of Raf protein (Yan et al., J. Biol. Chem., 273:24052-6 (1998)).

Among RAS isoforms, inhibition of the farnesylation of KRas protein and NRas protein leads to their geranylgeranylation and unchanged membrane localization. HRas protein however, cannot be geranylgeranylated and its membrane localization and cellular function is suppressed by farnesyltransferase inhibitors (FTIs) (Whyte et al., J. Biol. Chem., 272:14459-64 (1997)). Consequently, HRAS-mutant and HRAS-driven tumors are highly sensitive to FTIs.

PIK3CA is the most commonly dysregulated oncogene in HNSCC and HRas-MAPK and PI3K pathways may be interdependent in squamous cell carcinomas (“SCC”). For example, HRas protein requires PI3Kα to transform squamous epithelial cells (Gupta et al., Cell, 129:957 (2007)) and helicase domain PIK3CA mutants must bind Ras to cause cancer (Zhao et al., PNAS 105:2652 (2008)). HRAS and PIK3CA gene expression are negatively correlated in the TCGA cohort (International Cancer Genome Consortium, Nat. Commun., 4:2873 (2013)).

Tipifarnib is a selective, potent, non-peptidomimetic, farnesyl transferase inhibitor (FTI). Tipifarnib has shown activity across indications in several clinical trials (advanced breast cancer, metastatic pancreatic cancer, melanoma, small cell lung cancer [SCLC], myelodysplastic syndromes [MDS], multiple myeloma, urothelial tract transitional cell carcinoma, colorectal cancer, and non-small cell lung cancer [NSCLC]) (See, e.g., Adjei et al., J. Clin. Oncol., 21:1760-6 (2003); Cohen et al., J. Clin. Oncol., 21:1301-6 (2003); Johnston et al., J. Clin. Oncol., 21:2492-9 (2003); Alsina et al., Blood, 103:3271-7 (2004); Heymach et al., Ann. Oncol., 15:1187-93 (2004); Kurzrock et al., J. Clin. Oncol., 22-1287-92 (2004); Rao et al., J. Clin. Oncol., 22:3950-7 (2004); Rosenberg et al., Cancer, 103:2035-41 (2005); Hong et al., J. Clin. Endocrinol. Metab., 96:997-1005 (2011); and Gajewski et al., J. Transl. Med., 10:246 (2012)). However, methods to predict increased likelihood of response were not apparent, as these trials occurred before the advent of personalized medicine approaches. Importantly, tipifarnib is well tolerated in humans as evidenced by over two decades of safety and tolerability data and has shown encouraging clinical activity in HRAS-mutant squamous cell carcinomas (M. Gilardi et al., Mol. Cancer Ther. 19, 1784-1796 (2020); A. L. Ho et al., J. Clin. Oncol. 39, 1856-1864 (2021)).

Tipifarnib has been shown in vitro and in vivo to be a potent and selective inhibitor of farnesyltransferase. Isoprenylation catalyzed by geranylgeranyltransferase type 1 is not sensitive to inhibition by tipifarnib. The mechanism of action of tipifarnib is context dependent and appears to include angiogenesis inhibition, induction of apoptosis, and direct antiproliferative effects. Data from patient-derived xenograft (PDX) models of HRAS-driven cancers suggested that tipifarnib modifies tumor-host interactions important to the malignant process (End et al., Cancer Res., 61:131-7 (2001)).

Alpelisib is a potent and selective inhibitor of phosphatidylinositol-3-kinase (PI3K) with inhibitory activity predominantly against PI3Kα. PIQRAY® (also referred to as alpelisib or BYL719; see PIQRAY®'s Jul. 20, 2021 label, available at https://www.accessdata.fda.gov/drugsatfda_docs/label/2021/212526Orig1s0041bl.pdf. Also posted on Novartis' website at https://www.novartis.us/sites/www.novartis.us/files/piqray.pdf) was approved by the United States Food and Drug Administration (“US FDA”) in 2019 for treating, in combination with fulvestrant, postmenopausal women, and men, with hormone receptor (HR)-positive, human epidermal growth factor receptor 2 (HER2)-negative, PIK3CA-mutated, advanced or metastatic breast cancer as detected by an FDA-approved test following progression on or after an endocrine-based regimen. In vivo, alpelisib inhibited the PI3K/Akt-signaling pathway and reduced tumor growth in xenograft models, including models of breast cancer (Fritsch et al., Mol. Cancer Ther., (2014) 13.5: 1117-1129) and HNSCC (Sheng et al., Cancer Res., 73:4261 (2013)). In HNSCC cell lines, the combination of alpelisib and cetuximab induced over 50% growth inhibition in 85% of the HNSCC lines tested, regardless of PIK3CA mutation status (Sheng et al., Cancer Res., 73:4261 (2013)). In mouse xenograft models, antitumor activity was observed with alpelisib, both as a single agent or in combination with cetuximab. Alpelisib has been further studied in preclinical models with HNSCC cell lines and has showed activity against mutated and wild type cells (Sheng et al., Cancer Res., 73:4261 (2013); Keam et al., Anticancer Res., 35:175-82 (2015); Rucci et al., Int. J. Cancer, 145:2100-06 (2019); Meister et al., Sci. Rep., 9:9130 (2019)). While alpelisib has shown some promise in HNSCC in a phase I setting, its single agent efficacy will likely be limited by feedback re-activation of PI3K or compensatory parallel pathways, necessitating the development of rational combination strategies.

Key nonclinical data and PDX models have indicated additive and most often synergistic effects of the combination of tipifarnib and alpelisib in HRAS- and/or PIK3CA-dependent tumors (International Application No. PCT/US2020/025149, published as WO 2020/205486), that when translated to the clinic would mean a significant new therapeutic option for what is estimated to be approximately 50% of R/M HNSCC patients.

There remains a need for the treatment of cancers, in particular, treatment of HNSCC. There also remains a need in the art to treat cetuximab-resistant HNSCC, for example to overcome cetuximab resistance in HNSCC patients treated with cetuximab, and even to prevent or slow the emergence of resistance to cetuximab in HNSCC cetuximab-naïve patients. There is also a need for therapeutic options with enhanced efficacy, increased durability of response, and/or faster onset of antitumor response, compared to existing therapies, or compared to a PI3K inhibitor, such as alpelisib, alone. There is also a need for effective therapies in relapsed or refractory settings, such as second or third line treatments. The methods and compositions provided herein, comprising a PI3K inhibitor, such as alpelisib, and tipifarnib, expand the treatment, management, and prevention of HNSCC and/or drug resistance development during treatment. The methods and compositions provided herein provide benefit to HNSCC patients beyond HRAS mutational status to include, for example, HRAS- and PIK3CA-dependent tumors. Additionally, the methods and compositions provided herein, mitigates one or more of the above-noted resistance problems including the interdependencies of key cellular pathways associated with HNSCC.

### 4. SUMMARY

HRAS-MAPK and PI3K-AKT-mTOR are important oncogenic pathways in squamous cell carcinomas (SCCs) including those of the head and neck (HNSCC). Although HRas protein mutations occur at a rate of ˜5% in HNSCCs, overexpression of wild-type HRas protein is present in up to 30% of HNSCC tumors, raising the possibility that some wild-type HRas protein HNSCCs may also display a degree of HRAS dependence. PI3Kα (the catalytic subunit of PI3K), another prominent driver in HNSCC, is activated by PIK3CA mutations or PIK3CA gene amplification in about 30% of HNSCC patients. Overexpression of mutant or wild-type (“WT”) HRAS can drive resistance to PI3K inhibition in PIK3CA-mutant HNSCC cells. Feedback reactivation of PI3K or compensatory parallel pathways limits the single agent efficacy of PI3K inhibitors, necessitating development of rational combination strategies. Cooperation and crosstalk of the HRas protein and PI3K pathways can drive tumor progression and resistance to targeted therapies in SCCs.

Provided herein is tipifarnib, which is an active farnesyltransferase inhibitor that blocks hyperactivated growth factor signaling at multiple nodes, including farnesylation-dependent proteins, such as HRas protein and Rheb protein, which depend on farnesylation for their activation and/or cellular localization (and as Rheb is a non-redundant TORC1 activator, indirectly blocks mTOR activity as well). The addition of tipifarnib to alpelisib, according to the methods disclosed herein, provides increased efficacy, provides durable pathway inhibition, and/or provides an enhanced tumor cell death, or combinations thereof, in both PIK3α- and HRAS-dysregulated models. By impacting both HRAS-MAPK and PI3K-AKT-mTOR signaling, two major pathways driving PI3Ki resistance following alpelisib exposure, the combination of tipifarnib with alpelisib, according to the methods disclosed herein, blocks the feedback (re)activation of MAPK and mTOR that drives alpelisib resistance and relapse.

The present disclosure provides for use of tipifarnib in combination with a phosphatidylinositol-3-kinase (PI3K) inhibitor, such as alpelisib, for treating or managing HNSCC in a patient, such as in a patient having dysregulated PIK3CA-dependent HNSCC. Without being limited by any theory, the use of the two agents in HNSCC patients provides a more effective therapy then the use of either single agent alone. Indeed, in some embodiments, the combined use of the two agents is synergistic. In some embodiments, the combination of the two agents, according to the methods disclosed herein, provides increased efficacy, provides increased durability of response, provides a more rapid onset of antitumor response, prevents or delays development of PI3K inhibitor or alpelisib resistance, or prevents or delays relapse or disease progression, or combinations thereof, as compared to use of either agent alone.

In another important embodiment, provided herein is the use of tipifarnib before, during, or after the administration of alpelisib, to mitigate or reduce cetuximab-resistance. For instance, the use of tipifarnib in combination with alpelisib, in treating HNSCC can mitigate, slow the progression of, or overcome cetuximab-resistance in an HNSCC patient currently being treated or previously treated with cetuximab.

In another important embodiment, provided herein is using the combination of tipifarnib and alpelisib, before cetuximab therapy and continuing the use of the combination during cetuximab therapy. These methods help to improve cetuximab therapy and mitigate or reduce cetuximab resistance.

In the embodiments disclosed herein, there are therapy cycles or dosing regimens where tipifarnib and the alpelisib, are administered in a certain manner, such as continuously, or with days of administration followed by days of rest (or non-administration) of tipifarnib.

In another important embodiment, provided herein are pharmaceutical compositions, kits, and related products that include tipifarnib and/or alpelisib.

### 5. BRIEF DESCRIPTION OF THE DRAWINGS

FIGS. 1A-1G: Combination treatment of tipifarnib and a second agent alpelisib (BYL719) inhibited tumor growth in HNSCC PDX Models. FIG. 1A: HN2579 model having an HRAS mutation (G12S); FIG. 1B: HN3504 model having an HRAS mutation and a PIK3CA mutation (K117L and H1047R, respectively); FIG. 1C: HN2593 model having high wild-type HRAS expression levels (referred to as HRAS^{WT-high}) and having a PIK3CA mutation (G118D); FIG. 1D: HN3067 model having high wild-type HRAS expression levels (HRAS^{WT-high}) and PIK3CA amplification (referred to as PIK3CA^{AMP}); FIG. 1E: HN2594 model having high wild-type HRAS expression levels (HRAS^{WT-high}) and wild-type PIK3CA expression (PIK3CA^{WT}); FIG. 1F: dosing regimens for administering tipifarnib, alpelisib, and vehicle in the five (5) dosing regimen groups in FIGS. 1A-E and G during a four (4) week (i.e., 28 day) cycle, including four simultaneous combination dosing regimen variations such as tipifarnib dosed BID for 1 week alternating with 1 week of rest in a 28-day cycle; and alpelisib dosed QAM each day of the 28-day cycle; FIG. 1G: HN3690 model having a PIK3CA mutation (E545K) and HRAS^{WT-high}.

FIGS. 2A-2D: FIG. 2A: Immunoblots of MAPK/PI3K pathway components and apoptotic markers in PIK3CA-mutant CAL33 cells treated with alpelisib (BYL-719) for 0, 1, 2, 6, and 24 hours in the absence or presence of tipifarnib (48-hour treatment). FIGS. 2B and 2D: Immunoblots of MAPK/PI3K pathway components and apoptotic markers in HRas high HNSCC cell line SCC9 cells (FIG. 2B) and PIK3CA copy gain BICR22 cells (FIG. 2D) treated with alpelisib (BYL-719) for 0, 1, and 24 hours in the absence or presence of tipifarnib (48-hour treatment). FIG. 2C: Combination of tipifarnib and alpelisib in PIK3CA-mutant CAL33 cell line compared to single agents or vehicle, wherein cytotoxicity was measured using a dye to evaluate the loss of membrane integrity on Incucyte live cell imager.

FIGS. 3A-3C: HSC3 cell line model without PIK3CA alteration or elevated HRAS activity treated with the tipifarnib-alpelisib combination. FIG. 3A: Immunoblot of the indicated signaling proteins in HSC3 cells following exposure. FIGS. 3B-3C: Apoptosis (Annexin V) (FIG. 3B) and cytotoxicity (DNA stain/loss of membrane integrity) (FIG. 3C) over time in HSC3 cells treated with DMSO, 250 nM alpelisib, 1 μM tipifarnib, or the combination for 72 hours measured via Incucyte live cell imaging. 100 nM staurosporine was used as a positive control. Data represent means+/−SD of three biological replicates.

FIG. 4: Tipifarnib treatment markedly diminished the lysosomal localization of Rheb protein. Density gradient ultracentrifugation was utilized to extract lysosomes from CAL33 cells treated with DMSO or tipifarnib. Lysosomes were lysed and subjected to immunoblot analysis alongside whole-cell lysate (WCL). LAMP1 is a lysosome-specific marker and was used in this assay.

FIGS. 5A-5B: FIG. 5A: Plot depicting the distribution of HRAS expression by primary tissue site from analysis of Tempus Explore Database. FIG. 5B: Venn diagram depicting the genetic alterations or overexpression of the HRAS gene and the PIK3CA gene in HNSCC from analysis of Tempus Explore Database (OE=overexpression; Amp=amplification).

FIGS. 6A-6D: HRAS immunohistochemistry (IHC) assay using HRAS (ARC0098) & Rabbit IgG Staining in H&N Cancer. FIG. 6A: Percent Score ≥3+: Plasma Membrane 100, Cytoplasmic 0; FIG. 6B: Percent Score ≥3+: Plasma Membrane 60, Cytoplasmic 0; FIG. 6C: Percent Score ≥3+: Plasma Membrane 0, Cytoplasmic 0; FIG. 6D: Negative Control.

FIG. 7: Graph illustrating distribution of HRAS overexpression from 46 evaluable H&N cancer samples: 35% were positive (16/46), with 50% or more cells having a Percent Score ≥3+ Plasma Membrane or Cytoplasmic, and 65% were negative (30/46), according to the HRAS immunohistochemistry (IHC) assay.

FIGS. 8A-8C: The combination of tipifarnib and alpelisib inhibits mTOR signaling and induces apoptosis and tumor regression in PIK3CA mutant cell line-derived xenograft tumors. Growth of CAL33 (FIG. 8A) and HSC3 (FIG. 8B) cell line-derived xenograft tumors treated with vehicle, tipifarnib, alpelisib, or the combination. FIG. 8C: Immunoblots of signaling proteins in CAL33 tumors treated with tipifarnib, alpelisib, or the combination.

FIGS. 9A-C: Combined, synchronous tipifarnib-alpelisib treatment robustly inhibited the growth of PIK3CA- and HRAS-dysregulated HNSCC patient-derived xenograft models. FIGS. 9A-C: Growth of HNSCC PDX tumors harboring PIK3CA mutations as indicated (FIG. 9A), PIK3CA amplification (AMP) (FIG. 9B), or HRAS overexpression (FIG. 9C).

FIGS. 10A-C: Results demonstrating toxicity and mechanistic impacts of tipifarnib and alpelisib treatment on various cell lines. FIG. 10A: Inhibition of spheroid growth of HRAS-mutant cell line UM-SCC-17B (Q61L) in the presence of tipifarnib and/or alpelisib; FIG. 10B: Immunoblot showing the combination of tipifarnib and alpelisib inhibited activity of phospho-S6K and S6 and induced PARP cleavage in the PIK3CA-mutant HNSCC cell line HSC2 (H1047R); FIG. 10C: Immunoblot of levels of mTOR substrate phosphorylation at baseline and after 1 h or 24 h of alpelisib exposure in RHEB- and HRAS-expression depleted CAL33 (PIK3CA-mutant, H1047R), BICR22 (PIK3CA copy gain), and SCC9 (HRAS high) cells.

### 6. DETAILED DESCRIPTION

Provided herein are novel methods of using active farnesyltransferase inhibitor tipifarnib in combination with a phosphatidylinositol-3-kinase (PI3K) inhibitor, such as alpelisib. More specifically, methods provided herein include administering (a) tipifarnib and (b) alpelisib, for treating or managing HNSCC. In some embodiments, the methods provided herein mitigates, slows the progression of, or overcomes cetuximab-resistance in an HNSCC patient currently being treated, or previously treated, with cetuximab.

Provided herein are pharmaceutical compositions comprising tipifarnib and a pharmaceutically acceptable carrier, diluent or excipient. Provided herein are pharmaceutical compositions comprising (a) tipifarnib and a pharmaceutically acceptable carrier, diluent or excipient, and (b) alpelisib.

Provided herein are pharmaceutical kit or packaging comprising (a) a pharmaceutical composition comprising tipifarnib and a pharmaceutically acceptable carrier, diluent or excipient, and (b) a pharmaceutical composition comprising alpelisib, and a pharmaceutically acceptable carrier, diluent or excipient. In some embodiments, the pharmaceutical kit or packaging further comprises instructions details a dosing regimen or a color-coded system that details a dosing regimen.

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

As used herein, and in the specification and the accompanying claims, the indefinite articles “a” and “an” and the definite article “the” include plural as well as single referents, unless the context clearly indicates otherwise.

As used herein, and unless otherwise specified, the terms “about” and “approximately,” when used in connection with doses, amounts, or weight percentages of ingredients of a composition or a dosage form, mean a dose, amount, or weight percent within 30%, within 20%, within 15%, within 10%, or within 5%, of the specified dose, amount, or weight percent.

As used herein, the term “HNSCC” refers to head and neck squamous cell carcinoma (HNSCC). Head and neck squamous cell carcinoma (HNSCC) is the seventh most common invasive carcinoma worldwide, with about 830,000 new diagnoses annually worldwide and 200,000 deaths per year worldwide, and about 54,000 new cases per year in the US. It is also the most common cancer in central Asia. HNSCC has 2 different etiologies and corresponding tumor types. The first subtype is associated with tobacco smoking and alcohol consumption, and unrelated to Human papillomavirus (HPV− or HPV negative). The second subtype is associated with infection with high-risk HPV (HPV+ or HPV positive). The second subtype is largely limited to oropharyngeal cancers. HPV+ tumors are distinct entity with better prognosis and may require differential treatments. Significant proportion of HNSCC, particularly oropharyngeal cancers, are caused by HPV infection. High-risk HPV subtype 16 accounts for 85% of all HPV+ tumors in HNSCC. P16 can be used as surrogate marker of HPV infection in HNSCC, particularly in the oropharynx. More accurate HPV testing is available and based on E6/E7 detection (Liang et al. Cancer Res., 2012; 72:5004-5013).

HPV+ HNSCCs show significantly lower EGFR expression levels than HPV− HNSCC. EGFR amplification only occurs in HPV− HNSCC. High EGFR gene copy number and protein expression are associated with poor clinical outcome in advanced HNSCC.

Currently, first-line therapy for recurrent/metastatic HNSCC include platinum-based doublet (e.g., cisplatin/5-FU or carboplatin/paclitaxel), optionally in combination with anti-EGFR antibody therapy (e.g., cetuximab, panitumumab, afatinib). Second-line therapy includes taxanes, methotrexate, and/or cetuximab. Anti-EGFR antibody therapy, such as cetuximab (a chimeric IgG1) or panitumumab can be used as a single agent, with chemotherapy (e.g., platinum/5-FU, cisplatin; see EXTREME study NCT00122460), or with radiation therapy. Despite high EGFR expression levels in HNSCC, the single-agent response rate for cetuximab is only 13% with stable disease (SD) rate of 33%, and there is currently no predictive biomarker available.

Drugs in development for HNSCC include those targeting PI3K pathway: BKM120 (buparlisib)+ cetuximab, alpelisib+ cetuximab, Temsirolimus+ cetuximab, and Rigosertib+ cetuximab. Other therapeutic options for HNSCC include immunotherapy, such as anti-PD1 or anti-PDL1 antibodies. While high cure rates have been achieved for localized and loco-regional disease using surgery, radiation, chemoradiation, and induction chemotherapy, survival rates for recurrent/metastatic diseases remain very poor, and better treatment options are necessary.

As used herein, the term “first-line therapy” refers to therapies for treating HNSCC that include the use of a platinum-based doublet chemotherapy (e.g., cisplatin or carboplatin, such as cisplatin/5-FU or carboplatin/paclitaxel), optionally in combination with anti-EGFR antibody therapy (e.g., cetuximab, panitumumab, afatinib). In some embodiments, the first-line therapy includes pembrolizumab monotherapy or pembrolizumab in combination with the platinum-based doublet chemotherapy. In some embodiments, the first-line therapy is in the context of a patient having R/M HNSCC or an HNSCC patient only having received a localized or loco-regional disease therapy. First-line therapy of a R/M HNSCC patient is the first time a patient is treated after recurrence or diagnosis of metastatic disease. (See Haddad, J. Natl. Compr. Canc. Netw., 18(7.5):982-984 (2020); https://doi.org/10.6004/jnccn.2020.5009 (“Haddad (2020)”); and Borcoman et al., Cancers 13, 2573 (2021), https://doi.org/10.3390/cancers13112573 (“Borcoman (2021)”).

As used herein, the term “second-line therapy” refers to therapies for treating HNSCC in a patient having R/M HNSCC, or for treating HNSCC wherein at least one prior treatment has failed to mitigate or reduce the severity of at least one symptom associated with the HNSCC in the patient. For example, a second-line therapy can include the use of taxanes, methotrexate, and/or cetuximab. Second-line therapy of a R/M HNSCC patient is treatment of the patient after they have progressed on or after their first-line treatment. (See Haddad (2020) and Borcoman (2021).)

As used herein, the term “anti-EGFR antibody therapy” refers to therapies for treating HNSCC that include cetuximab (a chimeric IgG1) or panitumumab used as a single agent, used with chemotherapy (e.g. platinum/5-FU, cisplatin; see EXTREME study NCT00122460), or used with radiation therapy. (See Haddad (2020) and Borcoman (2021).)

As used herein, the term “immunotherapy” refers to therapies for treating HNSCC that include the use of anti-PD1 or anti-PDL1 antibodies. (See Haddad (2020) and Borcoman (2021).)

As used herein, the terms “localized regional disease therapy,” “loco-regional disease therapy,” “localized regional disease therapies” or “loco-regional disease therapies” refer to therapies for treating HNSCC that include the use of surgery, radiation, chemoradiation, or induction chemotherapy, or combinations thereof. (See Haddad (2020) and Borcoman (2021).)

The term “isomer” as used herein comprises a stereoisomer or tautomer as defined herein. As used herein, the term “stereoisomers” is understood to mean isomers that differ only in the way the atoms are arranged in space. As used herein, the term “isomer” includes any and all geometric isomers and stereoisomers. For example, “isomers” include geometric double bond cis- and trans-isomers, also termed E- and Z-isomers; R- and S-enantiomers; diastereomers, (d)-isomers and (l)-isomers, racemic mixtures thereof, and other mixtures thereof, as falling within the scope of this disclosure.

The term “isotopologue” refers to isotopically-enriched compounds that are identical to those recited herein, but for the fact that one or more atoms are replaced by an atom having an atomic mass or mass number different from the atomic mass or mass number usually found in nature. Unless otherwise stated, structures depicted herein are also meant to include compounds which differ only in the presence of one or more isotopically enriched atoms. Examples of isotopes that can be incorporated into compounds described herein include isotopes of hydrogen or carbon, such as ²H (deuterium) or ¹⁴C, respectively, each of which is also within the scope of this description. When the compounds are enriched with deuterium, the deuterium-to-hydrogen ratio on the deuterated atoms of the molecule substantially exceeds the naturally occurring deuterium-to-hydrogen ratio.

As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Remington's Pharmaceutical Sciences, 18th eds., Mack Publishing, Easton PA (1990) or Remington: The Science and Practice of Pharmacy, 19th eds., Mack Publishing, Easton PA (1995). Pharmaceutically acceptable salts of the compounds provided herein include those derived from suitable inorganic and organic acids and bases, such as suitable inorganic and organic addition acids and bases.

As used herein, the term “pharmaceutically acceptable carrier, excipient or diluent” means a carrier, excipient or diluent approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which a therapeutic agent is administered. Such pharmaceutical carriers can be sterile liquids, such as water and oils, including those of petroleum, animal, vegetable or synthetic origin, such as peanut oil, soybean oil, mineral oil, sesame oil, and the like. Water is a specific carrier for intravenously administered pharmaceutical compositions. Saline solutions and aqueous dextrose and glycerol solutions can also be employed as liquid carriers, particularly for injectable solutions. For example, the term pharmaceutically acceptable carrier, excipient, or diluent includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active ingredient, its use in the therapeutic compositions as disclosed herein is contemplated. Supplementary active ingredients can also be incorporated into the pharmaceutical compositions. Examples of excipients that can be used in oral dosage forms provided herein include, but are not limited to, binders, fillers, disintegrants, and lubricants.

As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. In some embodiments, the solvate is a hydrate. Pharmaceutically acceptable solvates and hydrates are complexes that, for example, can include 0.1, 0.25, 0.50, 0.75, or 1 solvent or water molecules, or can include 1 to about 100, or 1 to about 10, or one to about 2, about 3 or about 4, solvent or water molecules. It will be understood that the term “compound” as used herein encompasses the compound and solvates of the compound, as well as mixtures thereof.

As used herein and unless otherwise indicated, the term “stereoisomer” or “stereoisomerically pure” means one stereoisomer of a compound that is substantially free of other stereoisomers of that compound. For example, a stereoisomerically pure compound having one chiral center will be substantially free of the opposite enantiomer of the compound. For example, a stereoisomerically pure tipifarnib is substantially free of S enantiomer tipifarnib. A stereoisomerically pure compound having two chiral centers will be substantially free of other diastereomers of the compound. A typical stereoisomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments provided herein, including mixtures thereof.

The use of stereoisomerically pure forms of such compounds, as well as the use of mixtures of those forms, are encompassed by the embodiments provided herein. For example, mixtures comprising equal or unequal amounts of the enantiomers of a particular compound may be used in methods and compositions provided herein. These isomers may be asymmetrically synthesized or resolved using standard techniques such as chiral columns or chiral resolving agents. See, e.g., Jacques, J., et al., (Wiley-Interscience, New York, 1981); Wilen, S. H., et al., Tetrahedron 33:2725 (1977); Eliel, E. L., Stereochemistry of Carbon Compounds (McGraw-Hill, N Y, 1962); Wilen, S. H., Tables of Resolving Agents and Optical Resolutions p. 268 (E. L. Eliel, Ed., Univ. of Notre Dame Press, Notre Dame, I N, 1972); Todd, M., Separation Of Enantiomers: Synthetic Methods (Wiley-VCH Verlag GmbH & Co. KGaA, Weinheim, Germany, 2014); Toda, F., Enantiomer Separation: Fundamentals and Practical Methods (Springer Science & Business Media, 2007); Subramanian, G. Chiral Separation Techniques: A Practical Approach (John Wiley & Sons, 2008); Ahuja, S., Chiral Separation Methods for Pharmaceutical and Biotechnological Products (John Wiley & Sons, 2011).

It should be noted that if there is a discrepancy between a depicted structure and a name for that structure, the depicted structure is to be accorded more weight.

As used herein and unless otherwise indicated, the term “therapeutically effective amount” or “effective amount” in connection with a compound means an amount capable of treating, preventing, or managing a disorder, disease, or condition, or symptoms thereof.

As used herein and unless otherwise indicated, the term “subject” to which administration is contemplated, can be an animal, including, but not limited to, a human (e.g., a male or female of any age group, such as an adult subject or an adolescent subject); primates (e.g., cynomolgus monkeys, rhesus monkeys), and/or other mammals, including commercially relevant mammals such as cattle, pigs, horses, sheep, goats, cats, dogs, rabbits, rodents, and/or birds. In some embodiments, the subject is a mammal. In some embodiments, the subject is a human. In some embodiments, the subject is an adolescent human. In some embodiments, the subject is an adult human. In some embodiments wherein the subject is a human, the subject is a smoker. In some embodiments, the subject is a non-smoker. In some embodiments, the subject is a non-smoker who had previously been a smoker. In some embodiments, the subject is a patient, for example, a human patient or a patient having cancer, such as a human patient having a cancer. In some embodiments, the subject is a human patient having HNSCC.

In some embodiments, the subject is a human patient having HNSCC that has been previously treated with first-line therapy, such as platinum-based doublet therapy (e.g., cisplatin/5-FU or carboplatin/paclitaxel), optionally in combination with anti-EGFR antibody therapy (e.g., cetuximab, panitumumab, afatinib). In some embodiments, the subject is a human patient having HNSCC that has been previously treated with second-line therapy, such as taxanes, methotrexate, and/or cetuximab. In some embodiments, the subject is a human patient having HNSCC that has been previously treated with anti-EGFR antibody therapy, such as cetuximab or panitumumab used as a single agent, used with chemotherapy (e.g., platinum/5-FU, cisplatin), or used with radiation therapy. In some embodiments, the subject is a human patient having HNSCC that has been previously treated with immunotherapy, such as anti-PD1 or anti-PDL1 antibodies. In some embodiments, the subject is a human patient having HNSCC that has been previously treated with localized or loco-regional disease therapies, such as surgery, radiation, chemoradiation, or induction chemotherapy, or combinations thereof. In some embodiments, the subject is a human HNSCC patient that has R/M HNSCC. In some embodiments, the subject is a human HNSCC patient that has received at least one prior treatment. In some embodiments, the subject is a human HNSCC patient that has received at least one prior treatment, and the at least one prior treatment has failed to treat the HNSCC, has failed to delay, halt, or prevent progression of the HNSCC, or has failed to mitigate or reduce the severity of at least one symptom associated with the HNSCC. In some embodiments, the subject is a human patient having HNSCC that has received at least one prior treatment. In some embodiments, the at least one prior treatment is a first-line therapy. In some embodiments, the at least one prior treatment is a second-line therapy. In some embodiments, the at least one prior treatment is an anti-EGFR antibody therapy. In some embodiments, the at least one prior treatment is an immunotherapy. In some embodiments, the at least one prior treatment is a localized or loco-regional disease therapy.

As used herein and unless otherwise indicated, the terms “treat,” “treating,” “treatment,” and “ameliorating” are used interchangeably herein, and means an alleviation, in whole or in part, of a disorder, disease or condition, such as HNSCC, or one or more of the symptoms associated with a disorder, disease, or condition, such as HNSCC, or slowing or halting of further progression or worsening of those symptoms, or alleviating or eradicating the cause(s) of the disorder, disease, or condition itself, such as HNSCC. In some embodiments, these terms refer to an approach for obtaining beneficial or desired results including, but not limited to, a therapeutic benefit or a prophylactic benefit. A therapeutic benefit resulting from the methods of treatment provided herein includes the eradication or amelioration of the underlying disorder, such as HNSCC, being treated, the eradication or amelioration of one or more of the physiological symptoms associated with the underlying disorder (e.g., HNSCC) such that an improvement is observed in the patient, notwithstanding that the patient can still be afflicted with the underlying disease or disorder (e.g., HNSCC). For example, when used in reference to a patient having HNSCC, refers to an action that reduces the severity of the HNSCC, or retards or slows the progression of the HNSCC, including (a) inhibiting the HNSCC growth, or arresting development of the HNSCC, and (b) causing regression of the HNSCC, or delaying or minimizing one or more symptoms associated with the presence of the HNSCC.

As used herein, the terms “prevention” and “preventing” refer to an approach for obtaining beneficial or desired results including, but not limited, to prophylactic benefit. For prophylactic benefit, the compounds and pharmaceutical compositions disclosed herein can be administered to a patient at risk of developing HNSCC, to a patient reporting one or more of the physiological symptoms of HNSCC, even though a diagnosis of the HNSCC, may not have been made, or to a patient in remission from HNSCC. A prophylactic benefit resulting from the methods of treatment provided herein includes delaying or eliminating the appearance of a disease or disorder (e.g., HNSCC), delaying or eliminating the onset of symptoms of a disease or disorder (e.g., HNSCC), slowing, halting, or reversing the progression of a disease or disorder (e.g., HNSCC), or any combination thereof.

As used herein and unless otherwise indicated, the term “relapsed” refers to a disorder, disease, or condition that responded to treatment (e.g., achieved a complete response) then had progression. The treatment can include one or more lines of therapy. For example, “relapsed” HNSCC may refer to HNSCC that has been previously treated with one or more lines of therapy. In one embodiment, the relapsed HNSCC is HNSCC that has been previously treated with one, two, three or four lines of therapy. In one embodiment, the relapsed HNSCC is HNSCC that has been previously treated with two or more lines of treatment.

As used herein and unless otherwise indicated, the term “refractory” refers to a disorder, disease, or condition that has not responded to prior treatment that can include one or more lines of therapy. In some embodiments, the disorder, disease, or condition has been previously treated one, two, three or four lines of therapy. In some embodiments, the disorder, disease, or condition has been previously treated with two or more lines of treatment, and has less than a complete response (CR) to most recent systemic therapy containing regimen. For example, the disorder, disease, or condition is HNSCC.

As used herein and unless otherwise indicated, the terms “dysregulated PIK3CA” or “PI3KCA dysregulation,” refers to tumors that are dependent upon PIK3CA due to an oncogenic alteration in the PI3K pathway, including, but not limited to, oncogenic PIK3CA mutations, oncogenic amplification of the PIK3CA gene, oncogenic copy gain of the PIK3CA gene, and loss of PTEN function, or combinations thereof.

As used herein and unless otherwise indicated, the term “PIK3CA alteration” refers to tumors that are dependent upon a modified PIK3CA gene, such as a mutated PIK3CA gene or an amplified PIK3CA gene.

As used herein and unless otherwise indicated, the term “copy gain” refers to amplification of a gene between diploid (n=2) and the designated cutoff for “amplification” of the particular gene (for example, n=4 or 5). For example, the designated cutoff for amplified PIK3CA gene may be n=4 or 5, and thus copy gain of the PIK3CA gene would cover n=2 up to n=4 or 5, respectively.

As used herein and unless otherwise indicated, the term “Duration of Response” or “DoR” is the time from achieving a response until relapse or disease progression. In some embodiments, DoR is the time from achieving a response ≥partial response (PR) until relapse or disease progression. In some embodiments, DoR is the time from the first documentation of a response until the first documentation of progressive disease or death. In some embodiments, DoR is the time from the first documentation of a response ≥partial response (PR) until to the first documentation of progressive disease or death.

As used herein and unless otherwise indicated, the term “Event-Free Survival” or “EFS” means the time from treatment onset until any treatment failure, including disease progression, treatment discontinuation for any reason, or death.

As used herein and unless otherwise indicated, the term “Overall Response Rate” or “ORR” means the percentage of patients who achieve a response. In some embodiments, ORR means the sum of the percentage of patients who achieve complete and partial responses. In some embodiments, ORR means the percentage of patients whose best response ≥partial response (PR).

As used herein and unless otherwise indicated, the term “Overall Survival” or “OS” means the time from treatment onset until death from any cause.

As used herein and unless otherwise indicated, the term “Progression Free Survival” or “PFS” means the time from treatment onset until tumor progression or death. In some embodiments, PFS means the time from the first dose of compound to the first occurrence of disease progression or death from any cause. In some embodiments, PFS rates are computed using the Kaplan-Meier estimates.

As used herein and unless otherwise indicated, the term “Time To Progression” or “TTP” means the time from treatment onset until tumor progression; TTP does not include deaths.

As used herein and unless otherwise indicated, the term “Time To Response” or “TTR” means the time from the first dose of compound to the first documentation of a response. In some embodiments, TTR means the time from the first dose of compound to the first documentation of a response partial response (PR).

As used herein, the terms “HRAS immunohistochemistry assay,” “HRAS immunohistochemistry (IHC) assay,” or “HRAS IHC assay,” refer to a method of determining HRAS protein expression level intensity and cellular location in a tumor tissue using IHC and an HRAS antibody. In certain embodiments, the HRAS IHC assay disclosed herein uses an antibody, such as a monoclonal antibody, to detect HRAS expression in a tumor tissue sample collected from a subject having or suspected of having HNSCC. In certain embodiments, the HRAS IHC assay disclosed herein can detect HRAS expression, such as HRAS overexpression, at a plasma membrane of a cell within a tumor tissue sample. In certain embodiments, the HRAS IHC assay disclosed herein can detect HRAS expression, such as HRAS overexpression, in the cytoplasm of a cell within a tumor tissue sample. In certain embodiments, the HRas protein detected is wild-type HRas protein. In certain embodiments, the tumor tissue sample is an HNSCC tissue sample, such as from a subject having or suspected of having HNSCC. An embodiment of the HRAS IHC assay is disclosed in Example 8.

In certain embodiments, the HRAS IHC assay disclosed herein can be performed to screen HNSCC patients for HRAS expression levels, and in particular, to determine if the patient has an HRAS-overexpressing HNSCC. In some examples, the HRAS IHC assay is performed on a paraffin-embedded tumor tissue sample collected from a subject having or suspected of having HNSCC. Tumor tissue samples are subjected to an immunohistochemistry method using an antibody, such as a monoclonal antibody, that has binding affinity for human HRAS in the tissue sample. Any method known in the art can be used to detect the monoclonal antibody bound to HRAS in the tumor tissue sample. In some examples, a dual link system can be used that detects the monoclonal antibody and generates a chemical reaction which can be visualized after incubation of the tissue in a chromogen solution containing 3,3′-diaminobenzidine (DAB). Optionally, the tumor tissue can be stained with hemotoxin and eosin (H&E) after performing the HRAS IHC assay. Staining the tumor tissue sample with H&E may be useful in assisting the viewer (e.g., a pathologist) in assessing HRAS protein expression.

In certain embodiments, the HRAS IHC assay disclosed herein can be subjected to scoring for HRAS expression. Scoring for HRAS expression includes assessing the number of tumor cells having HRAS staining (i.e., HRAS protein expression). HRAS staining is recorded at a corresponding differential intensity on a four-point semi-quantitative scale (0, 1+, 2+, 3+). On this scale: 0=null, negative or non-specific staining, 1+=low or weak staining, 2+=medium or moderate staining, and 3+=high or strong staining. Percentages of cells having the differential intensities of HRAS expression can be used to determine Percent Scores and H-Scores. H-Scores are calculated by summing the percentage of cells with intensity of expression (positive staining) multiplied by their corresponding differential intensity on a four-point semi-quantitative scale (0, 1+, 2+, 3+). Thus, H-scores range from 0 to 300. Percent Scores are calculated by summing the percentages of intensities at either ≥1+, ≥2+ or ≥3+. Thus, percent scores range from 0 to 100. In some aspects, Plasma Membrane Percent Scores can be determined by summing the percentages of tumor cells having plasma membrane HRAS staining intensities at either ≥1+, ≥2+ or ≥3+. In other aspects, Cytoplasmic Percent Scores can be determined by summing the percentages of tumor cells having cytoplasm HRAS staining intensities at either ≥1+, ≥2+ or ≥3+.

In some embodiments, the HRAS IHC assays disclosed herein can be used to determine if an HNSCC is an HRAS-overexpressing HNSCC. In some embodiments, an HNSCC is determined to be an HRAS-overexpressing HNSCC when 50% or more of the stained HNSCC cells in a tissue sample from the HNSCC have a Plasma Membrane Percent Score of ≥3+. In some embodiments, an HNSCC is determined to be an HRAS-overexpressing HNSCC when 50% or more of the stained HNSCC cells in a tissue sample from the HNSCC have a Cytoplasmic Percent Score of ≥3+, according to the HRAS IHC assay.

### 6.1 Compounds

The methods provided herein include administering (a) a farnesyltransferase inhibitor and (b) a phosphatidylinositol-3-kinase (PI3K) inhibitor to a subject. In some embodiments, the farnesyltransferase inhibitor is a selective farnesyltransferase inhibitor, such as a compound that selectively inhibits farnesyltransferase with greater potency (lower IC₅₀ value) relative to the level of inhibition of geranylgeranyl transferase type-1. In some embodiments, the farnesyltransferase inhibitor or selective farnesyltransferase inhibitor is tipifarnib. In some embodiments, the PI3K inhibitor is a PI3Kα inhibitor. In a preferred embodiment, the PI3K inhibitor is alpelisib.

In some embodiments, the farnesyltransferase inhibitor or selective farnesyltransferase inhibitor is (R)-6-[amino(4-chlorophenyl)(1-methyl-1H-imidazol-5-yl)methyl]-4-(3-chlorophenyl)-1-methyl-2(1H)-quinolinone (referred to herein as tipifarnib; or Compound 1) of the following formula.

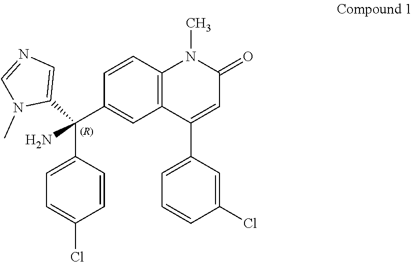

Tipifarnib is a non-peptidomimetic farnesyltransferase inhibitor (Thomas et al., Biologics, 1:415-424 (2007)) that competitively inhibits the CAAX peptide binding site of farnesyltransferase (“FTase”) and is a potent and selective inhibitor of farnesylation. Tipifarnib is not an inhibitor of geranylgeranyltransferase type 1.

In some embodiments, the PI3K inhibitor is selected from the group comprising, but is not limited to, alpelisib (PIQRAY®; BYL719), AMG319, AZD8168, AZD8835, buparlisib, B591, CH5132799, copanlisib (aliqopa), delalisib (zydelig), duvelisib (copiktra), eganelisib, GSK2636771, leniolisib, linperlisib, parsaclisib, pictilisib, pilaralisib, RIDR-PI-103, serabelisib, sonolisib, taselisib, tenalisib, TG-100-115, umbralisib, zandelisib, or ZSTK474, or a pharmaceutically acceptable form thereof. In some embodiments, the PI3K inhibitor, for example, a PI3Kα inhibitor, compound used in the methods provided herein is (2S)—N¹-[4-Methyl-5-[2-(2,2,2-trifluoro-1,1-dimethylethyl)-4-pyridinyl]-2-thiazolyl]-1,2-pyrrolidinedicarboxamide (referred to herein as alpelisib, PIQRAY®, BYL719, or Compound 2) of the following formula:

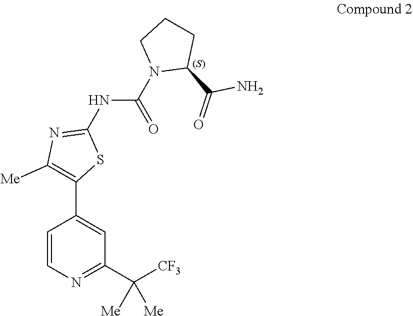

### 6.2 Pharmaceutical Compositions, Kits and Packaging

In some embodiments, provided herein is a pharmaceutical composition comprising tipifarnib and a pharmaceutically acceptable carrier, diluent or excipient. In some embodiments, provided herein is a pharmaceutical composition comprising alpelisib and a pharmaceutically acceptable carrier, diluent or excipient. In some embodiments, provided herein is a pharmaceutical composition comprising tipifarnib and alpelisib, and a pharmaceutically acceptable carrier, diluent or excipient.

In some embodiments, the pharmaceutical composition comprises tipifarnib and a pharmaceutically acceptable carrier, diluent, or excipient. For example, in some embodiments, the pharmaceutical composition comprises 1-1000 mg of tipifarnib such as an amount selected from the group consisting of 1-5 mg, 1-10 mg, 1-25 mg, 1-50 mg, 1-75 mg, 1-100 mg, 1-300 mg, 1-600 mg, 1-900 mg, 50-75 mg, 50-100 mg, 50-150 mg, 50-200 mg, 50-250 mg, 50-300 mg, 50-600 mg, 50-900 mg, 100-300 mg, 200-400 mg, 300-600 mg, 300-900 mg, 400-600 mg, 500-700 mg, 600-900 mg, and 800-1000 mg of tipifarnib. In some embodiments, the pharmaceutical composition comprises an amount selected from the group consisting of about 1 mg, about 5 mg, about 25 mg, about 50 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 650 mg, about 700 mg, about 750 mg, about 800 mg, about 850 mg, about 900 mg, about 950 mg, and about 1000 mg of tipifarnib. In some embodiments, the pharmaceutical composition comprises about 150 mg of tipifarnib. In some embodiments, the pharmaceutical composition comprises about 300 mg of tipifarnib. In some embodiments, the pharmaceutical composition comprises about 450 mg of tipifarnib. In some embodiments, the pharmaceutical composition comprises about 600 mg of tipifarnib. In some embodiments, the pharmaceutical composition comprises about 900 mg of tipifarnib.

In some embodiments, the pharmaceutical composition comprising the tipifarnib is formulated in a tablet, such as a film-coated tablet. In some embodiments, the pharmaceutical composition comprising the tipifarnib is formulated in a capsule. In some embodiments, the pharmaceutical composition comprising the tipifarnib further comprises an excipient. In some embodiments, the excipient is selected from the group consisting of lactose monohydrate, maize starch, hypromellose, microcrystalline cellulose, crospovidone, colloidal anhydrous silica, and magnesium stearate. In some embodiments, the pharmaceutical composition comprising the tipifarnib is formulated in a tablet. In some embodiments, the tablet comprises a coating. In some embodiments, the coating comprises polyvinyl alcohol, titanium dioxide, polyethylene glycol, and talc. In some embodiments, the pharmaceutical composition comprising the tipifarnib formulated in a tablet can be mixed with different media. In some embodiments, the media is selected from the group consisting of water, Ensure®, a protein shake, orange juice, apple juice, and tomato juice.

In some embodiments, the pharmaceutical composition comprises alpelisib and a pharmaceutically acceptable carrier, diluent or excipient. For example, in some embodiments, the pharmaceutical composition comprises 10-400 mg of alpelisib, such as an amount selected from the group consisting of 10-300 mg, 10-200 mg, 10-150 mg, 10-100 mg, 10-50 mg, 25-400 mg, 25-300 mg, 25-200 mg, 25-150 mg, 25-100 mg, 25-50 mg, 50-400 mg, 50-300 mg, 50-200 mg, 50-150 mg, 50-100 mg, 100-400 mg, 100-300 mg, 100-200 mg, 150-250 mg, 175-225 mg, 200-400 mg, or 200-300 mg of alpelisib. In some embodiments, the pharmaceutical composition comprises about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 260 mg, about 270 mg, about 275 mg, about 280 mg, about 290 mg, about 300 mg, about 325 mg, about 350 mg, about 375 mg or about 400 mg of alpelisib.

In some embodiments, the pharmaceutical composition comprises about 25 mg, about 50 mg, about 100 mg, about 150 mg, about 200 mg, about 250 mg, or about 300 mg of alpelisib. In some embodiments, the pharmaceutical composition comprises about 50 mg, about 150 mg, or about 200 mg of alpelisib. In some embodiments, the pharmaceutical composition comprises about 50 mg of alpelisib. In some embodiments, the pharmaceutical composition comprises about 150 mg of alpelisib. In some embodiments, the pharmaceutical composition comprises about 200 mg of alpelisib.

In some embodiments, the pharmaceutical composition comprising the alpelisib is formulated in a tablet, such as a film-coated tablet. In some embodiments, the pharmaceutical composition comprising the alpelisib further comprises an excipient. In some embodiments, the excipient is selected from the group consisting of hypromellose, magnesium stearate, mannitol, microcrystalline cellulose, and sodium starch glycolate. In some embodiments, the pharmaceutical composition comprising the alpelisib is formulated in a tablet. In some embodiments, the tablet comprises a coating. In some embodiments, the coating comprises hypromellose, iron oxide black, iron oxide red, macrogol/polyethylene glycol (PEG) 4000, talc, and titanium dioxide.

In some embodiments, the pharmaceutical compositions provided herein comprise a pharmaceutically acceptable carrier, diluent or excipient in an amount sufficient to exert a therapeutically useful effect in the absence of undesirable side effects on the patient treated.

In some embodiments, the pharmaceutical compositions are provided for administration to a subject in unit dosage forms, such as tablets, capsules, microcapsules, pills, powders, granules, troches, suppositories, injections, syrups, patches, creams, lotions, ointments, gels, sprays, sterile parenteral solutions or suspensions, and oral solutions or suspensions, and oil water emulsions containing suitable quantities of the compounds or pharmaceutically acceptable salts thereof. In some embodiments, the pharmaceutical compositions provided herein are in the form of a tablet, In some embodiments, the pharmaceutical compositions provided herein are in the form of a capsule, In some embodiments, the capsules contain a compound provided herein without an additional carrier, excipient or vehicle. Typically the compound disclosed herein is formulated into pharmaceutical compositions using techniques and procedures well known in the art (see, e.g., Ansel's Pharmaceutical Dosage Forms and Drug Delivery Systems, Twelfth Edition 2021). In some embodiments, the pharmaceutical compositions are formulated and administered in unit dosage forms or multiple dosage forms. Such dosage forms contain predetermined amounts of active ingredients, and may be prepared by methods of pharmacy well known to those skilled in the art. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

The pharmaceutical compositions provided herein may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens may be adjusted over time according to the individual need and the professional judgment of the person administering or supervising the administration of the pharmaceutical compositions, and that the concentration ranges set forth herein are exemplary only and are not intended to limit the scope or practice of the claimed pharmaceutical compositions.

The pharmaceutical compositions are intended to be administered by a suitable route, including but not limited to orally, parenterally, rectally, topically and locally. For oral administration, capsules and tablets can be formulated. The pharmaceutical compositions are in liquid, semi-liquid or solid form and are formulated in a manner suitable for each route of administration. In one embodiment, when administered orally, a compound provided herein is administered with a meal and water. In another embodiment, the compound provided herein is dispersed in water or juice (e.g., apple juice or orange juice) and administered orally as a solution or a suspension. In one embodiment, a compound provided herein is administered when the subject is fed. In one embodiment, a compound provided herein is administered when the subject is fed with high-fat and/or high-calorie food. In one embodiment, a compound provided herein is administered when the subject is fed with FDA-standard high-fat high-calorie breakfast. In one embodiment, a compound provided herein is administered when the subject is fasted. In one embodiment, a compound provided herein is administered after the subject has an at least 8-hour overnight fast. In one embodiment, a compound provided herein is administered with or without food.

The pharmaceutical compositions provided herein can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. In some embodiments, the pharmaceutical compositions provided herein are administered orally.

In some embodiments, the pharmaceutical compositions provided herein can be delayed or prolonged by proper formulation. For example, a slowly soluble pellet of the compound provided herein can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the compound provided herein in oily or emulsified vehicles that allow it to disperse slowly in the serum.

In some embodiments, provided herein is a pharmaceutical kit comprising (a) tipifarnib and (b) alpelisib. In some embodiments, provided herein is a pharmaceutical kit comprising (a) a pharmaceutical composition comprising tipifarnib and a pharmaceutically acceptable carrier, diluent or excipient, and (b) a pharmaceutical composition comprising alpelisib and a pharmaceutically acceptable carrier, diluent or excipient. In some embodiments, the pharmaceutical kit further comprises instructions details a dosing regimen for administering tipifarnib and administering the alpelisib for one or more cycles. In some embodiments, the pharmaceutical kit further comprises a color-coded system that details a dosing regimen for administering tipifarnib and administering the alpelisib for one or more cycles. In some embodiments, the pharmaceutical kit is a pharmaceutical packaging.

In some embodiments, the pharmaceutical kit or the pharmaceutical packaging further comprises instructions for administering the contents of the kit to a subject having HNSCC. For example, in some embodiments, the instructions may be color-coded detailing with one color the dosing regimen for administering tipifarnib during a 28-day treating cycle, such as administering once or twice per day on days 1-7, on days 1-7 and 15-21, on days 1-21, or each day of a 28-day treatment cycle, while detailing with a different color the dosing regimen for administering the alpelisib during a 28-day treating cycle, for example, administering the alpelisib once or twice per day on each day of a 28-day treatment cycle, such as administering the alpelisib once per day on each day of a 28-day treatment cycle. For example, in some embodiments, the instructions may be color-coded detailing an escalation dosing period, a reduction dosing period, or a loading dosing cycle, for administering tipifarnib. For example, in some embodiments, the instructions may be color-coded detailing an escalation dosing period or reduction dosing period for administering the alpelisib.

### 6.3 Methods, Dosing Regimens and Schedules

### 6.3.1 Therapeutic Uses and Methods

In some embodiments, the method provided herein is a method of treating HNSCC in a subject, comprising administering to the subject (a) tipifarnib and (b) a PI3K inhibitor. In some embodiments, the method of treating provided herein comprises administering to the HNSCC subject (a) a therapeutically effective amount of tipifarnib and (b) a therapeutically effective amount of a PI3K inhibitor. In some embodiments, the method of treating provided herein comprises administering to the HNSCC subject (a) a pharmaceutical composition comprising a therapeutically effective amount of tipifarnib and a pharmaceutically acceptable carrier, diluent or excipient, and (b) a pharmaceutical composition comprising a therapeutically effective amount of a PI3K inhibitor, and a pharmaceutically acceptable carrier, diluent or excipient. In some embodiments, the PI3K inhibitor is an PI3Kα inhibitor. In some embodiments, the PI3Kα inhibitor is alpelisib. In some embodiments, the method of treating provided herein comprises administering to the HNSCC subject tipifarnib before, after, or simultaneously with the alpelisib during one or more cycles, such as one or more 28-day cycles. In some embodiments, the method of treating further comprises administering to the HNSCC subject cetuximab, such as a pharmaceutical composition comprising cetuximab and a pharmaceutically acceptable carrier, diluent or excipient. In some embodiments, the method of treating HNSCC prevents or delays the emergence of cetuximab-resistance in a cetuximab-naïve HNSCC subject. In some embodiments, the method of treating HNSCC mitigates, slows the progression of, or overcomes, cetuximab-resistance in the HNSCC subject who is currently being treated with (or was previously treated with) cetuximab. In some embodiments, the method of treating is for a cetuximab-resistant HNSCC.

In some embodiments, the method provided herein is a method of mitigating, slowing the progression of, or overcoming cetuximab-resistance in a HNSCC subject that is currently being, or was previously, treated with cetuximab, comprising administering to the HNSCC subject (a) tipifarnib (or a pharmaceutical composition comprising the same), before, after, or simultaneously with a PI3K inhibitor, for example, a PI3Kα inhibitor, such as alpelisib, during one or more cycles, such as one or more 28-day cycles. In some embodiments, the subject is currently being treated with cetuximab. In some embodiments, the subject was previously treated with cetuximab. In some embodiments, the subject was previously (and is no longer being) treated with cetuximab. In some embodiments, the method further comprises administering to the HNSCC subject cetuximab, such as a pharmaceutical composition comprising cetuximab and a pharmaceutically acceptable carrier, diluent or excipient.

In some embodiments, the method provided herein is a method of preventing or delaying emergence of cetuximab-resistance in a cetuximab-naïve HNSCC subject, comprising administering to the HNSCC subject (a) tipifarnib (or a pharmaceutical composition comprising the same), before, after, or simultaneously with a PI3K inhibitor, for example, a PI3Kα inhibitor, such as alpelisib (or a pharmaceutical composition comprising the same), during one or more cycles, such as one or more 28-day cycles. In some embodiments, the method further comprises administering to the HNSCC subject cetuximab, such as a pharmaceutical composition comprising cetuximab and a pharmaceutically acceptable carrier, diluent or excipient.

In some embodiments, the subject has or suffers from HNSCC. In some embodiments, the subject has symptoms associated with HNSCC. In some embodiments, the subject is diagnosed as having HNSCC. In some embodiments, the subject is a cetuximab-naïve subject. In some embodiments, the subject is a cetuximab-naïve subject for HNSCC. In some embodiments, the subject is a previously treated HNSCC subject, such as a subject that has been treated previously with cetuximab. In some embodiments, the subject is currently being treated with cetuximab. In some embodiments, the HNSCC subject is a cetuximab-resistant HNSCC subject. In some embodiments, the subject is a HNSCC subject in remission. In some embodiments, the HNSCC is early stage HNSCC, metastatic HNSCC, advanced HNSCC, relapsed HNSCC, refractory HNSCC, or recurrent HNSCC. In some embodiments, the HNSCC is early stage HNSCC. In some embodiments, the HNSCC is metastatic HNSCC or advanced HNSCC. In some embodiments, the HNSCC is relapsed HNSCC or refractory HNSCC. In some embodiments, the HNSCC is recurrent and/or metastatic (R/M) HNSCC. In some embodiments, the HNSCC is human papillomavirus (HPV)-negative HNSCC. In some embodiments, the HNSCC is a solid tumor. In some embodiments, the subject is a mammal, for example, a human, such as a human patient having HNSCC.

In some embodiments, the subject is diagnosed as having HNSCC or having symptoms associated with HNSCC. In some embodiments, the subject may be diagnosed by one skilled in the art, for example, by analysis of plasma or a tissue biopsy, such as a tumor tissue biopsy, from the subject.

In some embodiments, the HNSCC subject has an HNSCC that is associated with a farnesylation-dependent protein. In some embodiments, the HNSCC subject has an HRAS-dependent HNSCC or an HRAS-mutant dependent HNSCC. In some embodiments, the HNSCC subject has a PIK3CA-dependent HNSCC. In some embodiments, the subject has an HNSCC that is dependent on HRAS and PIK3CA. In some embodiments, the HRAS-dependent and PIK3CA-dependent HNSCC overexpresses wild-type HRas protein. In some embodiments, the HNSCC is an HRAS-mutant dependent and PIK3CA-dependent HNSCC. In some embodiments, the HRAS-dependent and PIK3CA-dependent HNSCC has a PIK3CA alteration or dysregulation. In some embodiments, the PIK3CA alteration or dysregulation is or comprises a PIK3CA mutation, a PIK3CA gene amplification, or a PIK3CA copy gain, or combinations thereof. Without being bound by any one theory, it is thought that the HRAS and PIK3CA cellular pathways are interdependent, such as they show codependency in HNSCC. There exists evidence that HRAS preferentially activates PI3K five-fold more efficiently than KRAS, while KRAS is a more efficient activator of Raf protein (Yan et al., J. Biol. Chem., 273: 24052-6 (1998)). Furthermore, mutant HRAS requires PI3K for activity, and is insufficient to be tumorigenic in isolation (Gupta et al., Cell, 129:957-68 (2007)). Similarly, mutant PI3K requires RAS in order to drive tumor biology (Zhao and Vogt, Oncogene, 27: 5486-96 (2008)).

In some embodiments, the HNSCC subject has an HNSCC that is associated with a farnesylation-dependent protein. For example, in some embodiments, the farnesylation-dependent protein associated with the HNSCC is an HRas protein, such as wild-type HRas protein or an HRas protein having a mutation. For example, in some embodiments, the farnesylation-dependent protein associated with the HNSCC is a Rheb protein. In some embodiments, the HNSCC subject has an HRAS-dependent HNSCC. In some embodiments, the HRAS-dependent HNSCC overexpresses wild-type HRas protein. In some embodiments, the HRAS-dependent HNSCC is an HRAS-mutant dependent HNSCC. For example, in some embodiments, the HNSCC is an HRAS-mutant dependent HNSCC, wherein the HRAS mutation is or comprises a modification in a codon that encodes an amino acid substitution at a specific position selected from a group consisting of G12, G13, Q61, Q22, K117, A146, and any combination thereof, in the corresponding mutant HRas protein. For example, in some embodiments, the HNSCC subject has an HRAS-mutant dependent HNSCC that has two or more, or three or more, HRAS mutations, wherein the HRAS mutation is or comprises a modification in a codon that encodes an amino acid substitution at a specific position selected from a group consisting of G12, G13, Q61, Q22, K117, A146, and any combination thereof, in the corresponding mutant HRas protein. Without being bound by any one theory, in some embodiments, tipifarnib inhibits the farnesylation of a farnesylation-dependent protein, for example, a Rheb protein or a HRas protein, such as an HRas protein having a mutation, in a cell, such as in a cell of a subject. In some embodiments, the cell is a mammalian cell. In some embodiments, the cell a human cell. In some embodiments, the inhibition of the farnesyltransferase present in the cell takes place in a subject suffering from HNSCC. In some embodiments, the HRAS-dependent HNSCC is further dependent on PIK3CA.

In some embodiments, the HNSCC subject has a PIK3CA-dependent HNSCC. In some embodiments, the PIK3CA-dependent HNSCC has a PIK3CA alteration or dysregulation. In some embodiments, the PIK3CA alteration or dysregulation is or comprises a PIK3CA mutation, a PIK3CA gene amplification, or a PIK3CA copy gain, or combinations thereof. In some embodiments, the PIK3CA alteration or dysregulation is a PIK3CA mutation. In some embodiments, the PIK3CA alteration or dysregulation is a PIK3CA gene amplification. In some embodiments, the PIK3CA alteration or dysregulation is a PIK3CA copy gain. In some embodiments, the PIK3CA-dependent HNSCC is a PIK3CA-mutant dependent HNSCC. For example, in some embodiments, the HNSCC is a PIK3CA-mutant dependent HNSCC, wherein the PIK3CA mutation is or comprises a modification in a codon that encodes an amino acid substitution at a specific position selected from a group consisting of R38, E39, E78, R88, R93, E103, P104, V105, G106, R108, E109, E110, K111, G118, P124, E218, V344, N345, D350, G364, E365, P366, C378, C420, P447, P449, H450, G451, E453, P471, P539, E542, E545, Q546, D549, E579, E600, C604, S629, V638, C901, G914, D939, E970, M1004, G1007, Y1021, T1025, D1029, E1037, M1043, N1044, H1047, G1049, A1066, and N1068, and any combination thereof, in the corresponding mutant PI3K protein. For example, in some embodiments, the HNSCC is a PIK3CA-mutant dependent HNSCC, wherein the PIK3CA mutation is or comprises a modification in a codon that encodes an amino acid substitution at a specific position selected from a group consisting of G118, C420, E542, E545, Q546, H1047, and any combination thereof, in the corresponding mutant PI3K protein. For example, in some embodiments, the HNSCC subject has a PIK3CA-mutant mediated HNSCC that has two or more, or three or more, PIK3CA mutations, wherein the PIK3CA mutation is or comprises a modification in a codon that encodes an amino acid substitution at a specific position selected from a group consisting of G118, C420, E542, E545, Q546, H1047, and any combination thereof, in the corresponding mutant PI3K protein. In some embodiments, the PIK3CA-dependent HNSCC comprises a mutant PI3K protein having an activating mutation, such as an activating mutation in the helicase domain or the kinase domain. In some embodiments, the PIK3CA-dependent HNSCC comprises a mutant PI3K protein having an activating mutation in the helicase domain, for example, in Exon 7 (e.g., C420R), or in Exon 9 (e.g., E542K, E545A, E545D, E545G, E545K, Q546E, or Q546R). In some embodiments, the PIK3CA-dependent HNSCC comprises a mutant PI3K protein having an activating mutation in the kinase domain, for example, in Exon 20 (e.g., H1047L, H1047R, or H1047Y). In some embodiments, the PIK3CA-dependent HNSCC is further dependent or associated with a farnesylation-dependent protein. In some embodiments, the PIK3CA-dependent HNSCC is further dependent on HRAS or mutated HRAS.

In some embodiments, the subject may be diagnosed as having an HNSCC that is associated with a farnesylation-dependent protein, an HRAS-dependent HNSCC, such as an HRAS-mutant dependent HNSCC, or a PIK3CA-dependent HNSCC, or combinations thereof, by detection methods known in the art. For example, in some embodiments, the subject may be diagnosed by common testing practices known in the art, such as by detection and/or analysis of plasma or a tissue biopsy with detection and/or analysis of plasma or a tissue biopsy with Next Gene Sequence (NGS) testing of tumor tissue, real time quantitative reverse transcription polymerase chain reaction (qRT-PCR) or immunohistochemistry (IHC).

Without being bound by any one theory, in some embodiments, inhibiting farnesylation of a farnesylation-dependent protein, for example a Rheb protein, an HRas protein, such as a wild-type HRas protein or an HRas protein having one or more mutations, by administering tipifarnib before, after, or simultaneously with a PI3K inhibitor, such as alpelisib, during one or more cycles, according to the methods provided herein, provides a synergistic benefit, such as a therapeutic benefit to the subject, relative to the administration of the individual agents.

In some embodiments, the methods disclosed herein of administering tipifarnib and alpelisib, are effective in treating HNSCC having wild-type HRAS, for example, having high levels of HRAS (“HRAS^{WT-high}”), such as having overexpression of wild-type HRas protein (high wild-type HRas protein expression levels; “HRas^{WT-high}”) and having wild-type PIK3CA (PIK3CA^{WT}) expression levels, for example, as in a PDX model, such as in HN2594, HN3411, and/or HN2576 model.

In some embodiments, the methods disclosed herein of administering tipifarnib and alpelisib, are effective in treating HNSCC having wild-type HRAS (e.g., HRAS^{WT-high}), such as having overexpression of wild-type HRas protein (HRas^{WT-high}) and having amplified PIK3CA (PIK3CA^{AMP}), for examples, having amplified expression levels of the corresponding PI3K protein, such as amplified expression levels of wild-type PI3K protein, for example, as in a PDX model, such as in HN3067 model (HRAS^{WT-high} and PIK3CA^{AMP}).

In some embodiments, the methods disclosed herein of administering tipifarnib and alpelisib, are effective in treating HNSCC having wild-type HRAS (e.g., HRAS^{WT-high}), such as having overexpression of wild-type HRas protein (HRas^{WT-high}) and having mutated PIK3CA (PIK3CA^{mutant}), such as having expression of mutated PI3K protein, for example, as in a PDX model, such as in HN2593 (HRAS^{WT-high} and PIK3CA^{G118D}) and HN3690 (HRAS^{WT-high} and PIK3CA^{E545K}) models. For example, in some embodiments, the PIK3CA mutation is or comprises a modification in a codon of the mutant PIK3CA gene encoding an amino acid at the specified position selected from a group consisting of G118, C420, E542, E545, Q546, H1047, and any combination thereof, to provide the resulting mutated PI3K protein, such as a mutated PI3K-α protein. In some embodiments, the PIK3CA gene mutation is or comprises PIK3CA^{G118D}, PIK3CA^{E545K}, or a combination thereof. In some embodiments, the PIK3CA mutated protein is or comprises PI3K-α G118D, PI3K-α E545K, or a combination thereof.

In some embodiments, the methods disclosed herein of administering tipifarnib and alpelisib, are effective in treating HNSCC having mutated HRAS and/or mutated HRas protein, for example, as in a PDX model, such as in HN1420 (HRAS A146 (mutated HRas protein A146T), PIK3CA WT), HN2581 (HRAS G13 (mutated HRas protein G13C), PIK3CA WT), HN2579 (HRAS G12 (mutated HRas protein G12S), PIK3CA WT), and HN3504 models (HRAS K117 (mutated HRas protein K117L), PIK3CA H1047R (mutated PI3K protein H1047R).

In some embodiments, the methods disclosed herein of administering tipifarnib and alpelisib, are effective in treating HNSCC having mutated HRas protein and having mutated PIK3CA expression levels as seen in PDX models, for example, in HN3504 model. In some embodiments, the HRas protein mutation is or comprises a modification in a codon of the mutant HRAS gene encoding an amino acid at the specified position to provide the resulting mutated HRas protein comprising or consisting of HRAS G12S, HRAS G13C, HRAS K117L, HRAS A146T, or a combination thereof. In some embodiments, the PI3KCA mutation is or comprises PIK3CA H1047R protein mutation.

In some embodiments, the methods disclosed herein provides a synergistic or therapeutic benefit to the HNSCC subject, for example. improves efficacy, such as suppressing tumor growth or inducing tumor regression, better than either compound therapy alone. In some embodiments, the methods provided herein improves efficacy, such as suppressing tumor growth or inducing tumor regression, better than the sum of each single compound therapy. In some embodiments, the methods provided herein delays, halts or prevents progression of HNSCC or HNSCC tumor growth. In some embodiments, the methods provided herein reduces HNSCC tumors, such as reduces a primary HNSCC tumor, delays the appearance of primary or secondary HNSCC tumors, slows the development of primary or secondary HNSCC tumors, decreases the occurrence of primary or secondary HNSCC tumors, or arrests HNSCC tumor growth. In some embodiments, the methods provided herein relieves HNSCC tumor-related symptoms. In some embodiments, the methods provided herein inhibits HNSCC tumor secreted factors. In some embodiments, the methods provided herein slows or decreases the severity of secondary effects associated with HNSCC. In some embodiments, the methods disclosed herein provide for improved efficacy, longer duration of response, or faster onset of antitumor efficacy, or combinations thereof, compared to treatment with the individual agents, standard of care therapies, other second- or third-line therapies, or no therapy. In some embodiments, the methods provided herein increases Time To Progression (TTP), Progression Free Survival (PFS), Event-free survival (EFS), Overall Survival (OS), overall response rate (ORR), duration of response (DoR), disease control rate (DCR; complete response (CR) plus partial response (PR) plus stable disease (SD)), rate of CR, or rate of SD, better than either compound therapy alone. In some embodiments, the methods provided herein increases TTP, PFS, EFS, OS, ORR, DoR, DCR, rate of CR, or rate of SD, better than first-line therapy. In some embodiments, the methods provided herein increases TTP, PFS, EFS, OS, ORR, DoR, DCR, rate of CR, or rate of SD, better than second-line therapy. In some embodiments, the methods provided herein increases TTP, PFS, EFS, OS, ORR, DoR, DCR, rate of CR, or rate of SD, better than anti-EGFR antibody therapy. In some embodiments, the methods provided herein increases TTP, PFS, EFS, OS, ORR, DoR, DCR, rate of CR, or rate of SD, better than immunotherapy. In some embodiments, the methods provided herein increases TTP, PFS, EFS, OS, ORR, DoR, DCR, rate of CR, or rate of SD, better than localized or loco-regional disease therapies. In some embodiments, the methods provided herein decreases time to response (TTR) better than either compound therapy alone. In some embodiments, the methods provided herein decreases TTR better than first-line therapy. In some embodiments, the methods provided herein decreases TTR better than second-line therapy. In some embodiments, the methods provided herein decreases TTR better than anti-EGFR antibody therapy. In some embodiments, the methods provided herein decreases TTR better than immunotherapy. In some embodiments, the methods provided herein decreases TTR better than localized or loco-regional disease therapies.

In some embodiments, the increased TTP, PFS, OS, EFS, ORR, DoR, DCR, rate of CR, or rate of SD, provided by the methods provided herein is independently 5%, 10%, 15%, 20%, 25%, 30%, 35%, 40%, 45%, 50%, 5 to 10%, 10 to 20%, 20 to 30%, 30 to 40%, 40 to 50%, 2 fold, 3 fold, or 4 fold, or more than 4 fold, better than either compound therapy alone, better than first-line therapy, better than second-line therapy, better than anti-EGFR antibody therapy, better than immunotherapy, or better than localized or loco-regional disease therapies. In some embodiments, the methods provided herein increases TTP better than either compound therapy alone, better than first-line therapy, better than second-line therapy, better than anti-EGFR antibody therapy, better than immunotherapy, or better than localized or loco-regional disease therapies. For example, in some embodiments, the methods provided herein increases TTP 5%, 10%, 15%, 20%, 25%, 30%, 35%, 40%, 45%, 50%, 5 to 10%, 10 to 20%, 20 to 30%, 30 to 40%, 40 to 50%, 2 fold, 3 fold, or 4 fold, or more than 4 fold. For example, in some embodiments, the methods provided herein increases PFS better than either compound therapy alone, better than first-line therapy, better than second-line therapy, better than anti-EGFR antibody therapy, better than immunotherapy, or better than localized or loco-regional disease therapies. For example, in some embodiments, the methods provided herein increases PFS 5%, 10%, 15%, 20%, 25%, 30%, 35%, 40%, 45%, 50%, 5 to 10%, 10 to 20%, 20 to 30%, 30 to 40%, 40 to 50%, 2 fold, 3 fold, or 4 fold, or more than 4 fold. In some embodiments, the method provided herein increases OS better than either compound therapy alone, better than first-line therapy, better than second-line therapy, better than anti-EGFR antibody therapy, better than immunotherapy, or better than localized or loco-regional disease therapies. For example, in some embodiments, the methods provided herein increases OS 5%, 10%, 15%, 20%, 25%, 30%, 35%, 40%, 45%, 50%, 5 to 10⁰/a, 10 to 20%, 20 to 30%, 30 to 40%, 40 to 50%, 2 fold, 3 fold, or 4 fold, or more than 4 fold. In some embodiments, the methods provided herein increases PFS and/or OS better than either compound therapy alone, better than first-line therapy, better than second-line therapy, better than anti-EGFR antibody therapy, better than immunotherapy, or better than localized or loco-regional disease therapies. In some embodiments, the methods provided herein increases EFS better than either compound therapy alone, better than first-line therapy, better than second-line therapy, better than anti-EGFR antibody therapy, better than immunotherapy, or better than localized or loco-regional disease therapies. For example, in some embodiments, the methods provided herein increases EFS 5%, 10%, 15%, 20%, 25%, 30%, 35%, 40%, 45%, 50%, 5 to 10%, 10 to 20%, 20 to 30%, 30 to 40%, 40 to 50%, 2 fold, 3 fold, or 4 fold, or more than 4 fold. In some embodiments, the methods provided herein increases ORR better than either compound therapy alone, better than first-line therapy, better than second-line therapy, better than anti-EGFR antibody therapy, better than immunotherapy, or better than localized or loco-regional disease therapies. For example, in some embodiments, the methods provided herein increases ORR 5%, 10%, 15%, 20%, 25%, 30%, 35%, 40%, 45%, 50%, 5 to 10%, 10 to 20%, 20 to 30%, 30 to 40%, 40 to 50%, 2 fold, 3 fold, or 4 fold, or more than 4 fold. In some embodiments, the methods provided herein increases DoR better than either compound therapy alone, better than first-line therapy, better than second-line therapy, better than anti-EGFR antibody therapy, better than immunotherapy, or better than localized or loco-regional disease therapies. For example, in some embodiments, the methods provided herein increases DoR 5%, 10%, 15%, 20%, 25%, 30%, 35%, 40%, 45%, 50%, 5 to 10%, 10 to 20%, 20 to 30%, 30 to 40%, 40 to 50%, 2 fold, 3 fold, or 4 fold, or more than 4 fold. In some embodiments, the methods provided herein increases DCR, or an individual component thereof, better than either compound therapy alone, better than first-line therapy, better than second-line therapy, better than anti-EGFR antibody therapy, better than immunotherapy, or better than localized or loco-regional disease therapies. For example, in some embodiments, the methods provided herein increases DCR, or an individual component thereof, 5%, 10%, 15%, 20%, 25%, 30%, 35%, 40%, 45%, 50%, 5 to 10%, 10 to 20%, 20 to 30%, 30 to 40%, 40 to 50%, 2 fold, 3 fold, or 4 fold, or more than 4 fold. In some embodiments, the methods provided herein increases rate of complete response (CR), or an individual component thereof, better than either compound therapy alone, better than first-line therapy, better than second-line therapy, better than anti-EGFR antibody therapy, better than immunotherapy, or better than localized or loco-regional disease therapies. For example, in some embodiments, the methods provided herein increases rate of CR, or an individual component thereof, 5%, 10%, 15%, 20%, 25%, 30%, 35%, 40%, 45%, 50%, 5 to 10%, 10 to 20%, 20 to 30%, 30 to 40%, 40 to 50%, 2 fold, 3 fold, or 4 fold, or more than 4 fold. In some embodiments, the methods provided herein increases rate of stable disease (SD), or an individual component thereof, better than either compound therapy alone, better than first-line therapy, better than second-line therapy, better than anti-EGFR antibody therapy, better than immunotherapy, or better than localized or loco-regional disease therapies. For example, in some embodiments, the methods provided herein increases rate of SD, or an individual component thereof, 5%, 10%, 15%, 20%, 25%, 30%, 35%, 40%, 45%, 50%, 5 to 10%, 10 to 20%, 20 to 30%, 30 to 40%, 40 to 50%, 2 fold, 3 fold, or 4 fold, or more than 4 fold. In some embodiments, the methods provided herein decreases TTR better than either compound therapy alone, better than first-line therapy, better than second-line therapy, better than anti-EGFR antibody therapy, better than immunotherapy, or better than localized or loco-regional disease therapies. For example, in some embodiments, the methods provided herein decreases TRR 5%, 10%, 15%, 20%, 25%, 30%, 35%, 40%, 45%, 50%, 5 to 10%, 10 to 20%, 20 to 30%, 30 to 40%, 40 to 50%, 2 fold, 3 fold, or 4 fold, or more than 4 fold.

In some embodiments, the methods provided herein delays the progression or the time to emergence of drug resistance (such as cetuximab-resistance). In some embodiments, the methods provided herein overcomes cetuximab-resistance. In some embodiments, the methods provided herein reduces the therapeutically effective amount of the alpelisib. In some embodiments, the methods provided herein reduces alpelisib-associated toxicity, such as reduces a toxicity selected from the group consisting of reduced glucose increase, creatinine increase, diarrhea, rash, lymphocyte count decrease, GGT increase, nausea, ALT increase, fatigue, hemoglobin decrease, lipase increase, decrease appetite, stomatitis, vomiting, weight decrease, calcium decrease, glucose decrease, a PTT prolonged, and alopecia, or a combination thereof. In some embodiments, the methods provided herein reduces the therapeutically effective amount of the cetuximab. In some embodiments, the methods provided herein reduces cetuximab-associated toxicity, such as reduces a toxicity selected from the group consisting of skin toxicity, including skin rash, dry skin, hair growth disorders, pruritus, nail changes, headache, and diarrhea. In some embodiments, the methods disclosed herein provides a combination of one or more of the above mentioned benefits.

In some embodiments, the methods provided herein mitigates or ameliorates one or more symptoms of HNSCC. In some embodiments, the methods provided herein retards the progression of, delays the time to emergence of, or overcomes drug resistance, such as cetuximab-resistance in the HNSCC subject. In some embodiments, the methods provided herein reduces or delays the risk of the HNSCC relapsing in said subject.

In some embodiments, the therapeutically effective amount of tipifarnib and/or the alpelisib, in the pharmaceutical composition, or the pharmaceutical kit or pharmaceutical packaging comprising the same, can depend on absorption, tissue distribution, metabolism, excretion rates of the active compound, the dosage schedule, amount administered, particular formulation as well as other factors known to those of skill in the art. The therapeutically effective amount may be determined empirically by testing the compounds in in vitro and in vivo systems described herein and then extrapolated therefrom for dosages for humans.

In some embodiments, the methods provided herein reduces the therapeutically effective amount of the alpelisib administered per day or administered per dose. For example, in some embodiments, the therapeutically effective amount of the alpelisib is reduced relative to the therapeutically effective amount required for alpelisib monotherapy, for example is reduced from 300 mg to 250 mg, 200 mg, 150 mg, or 75 mg, once per day, such as from 300 mg to 200 mg, from 250 mg to 200 mg, from 200 mg to 150 mg, or from 150 mg to 100 mg, once per day. In some embodiments, a reduction in the therapeutically effective amount of the alpelisib administered per day or administered per dose reduces the incidence or severity of, or the risk of, one or more alpelisib-associated toxicities. In some embodiments, the methods provided herein reduces alpelisib-associated toxicity or the risk thereof, such as reduces a toxicity selected from the group consisting of reduced glucose increase, creatinine increase, diarrhea, rash, lymphocyte count decrease, GGT increase, nausea, ALT increase, fatigue, hemoglobin decrease, lipase increase, decrease appetite, stomatitis, vomiting, weight decrease, calcium decrease, glucose decrease (hypoglycemia), aPTT prolonged, and alopecia, or a combination thereof. In some embodiments, the methods provided herein delays emergence of alpelisib-resistance. In some embodiments, the methods provided herein unexpectedly delays emergence of alpelisib-resistance. In some embodiments, the delay in emergence of alpelisib-resistance comprises weeks, months, or years.

In some embodiments, the methods provided herein reduces the therapeutically effective amount of the cetuximab administered per day or administered per dose. In some embodiments, the methods provided herein reduces cetuximab-associated toxicity, such reduces a toxicity selected from the group consisting of skin toxicity, including skin rash, dry skin, hair growth disorders, pruritus, nail changes, headache, and diarrhea or combinations thereof. In some embodiments, the methods provided herein delays emergence of cetuximab-resistance. In some embodiments, the methods provided herein unexpectedly delays emergence of cetuximab-resistance. In some embodiments, the delay in emergence of cetuximab-resistance comprises weeks, months, or years.

### 6.3.2 Doses and Regimens

In some embodiments, the methods provided herein comprises administering to the subject (a) tipifarnib (or a pharmaceutical composition comprising the same), and (b) a PI3K inhibitor (or a pharmaceutical composition comprising the same). In some embodiments, the methods provided herein comprises administering to the subject (a) tipifarnib (or a pharmaceutical composition comprising the same), and (b) a dose amount of a PI3K inhibitor (or a pharmaceutical composition comprising the same). In some embodiment, the administration is concurrent, sequential, continuous, intermittent, or in cycles. In some embodiments, PI3K inhibitor is a PI3Kα inhibitor, such as alpelisib.

In some embodiments, tipifarnib is administered to the subject according to the methods provided herein at a dose of 1-2400 mg per day. In some embodiments, the dose of tipifarnib is selected from the group consisting of 1-5 mg, 1-10 mg, 1-25 mg, 1-50 mg, 1-75 mg, 1-100 mg, 1-300 mg, 1-600 mg, 1-1200 mg, 1-2400 mg, 50-100 mg, 50-150 mg, 75-100 mg, 100-200 mg, 125-200 mg, 150-300 mg, 200-250 mg, 200-400 mg, 300-600 mg, 250-500 mg, 400-600 mg, 500-750 mg, 600-900 mg, 700-900 mg, 800-1000 mg, 900-1200 mg, 1100-1300 mg, 1200-1600 mg, 1200-2400 mg, 1500-2000 mg, 1500-2400 mg, 1800-2400 mg and 2000-2400 mg per day. In some embodiments, the dose of tipifarnib is selected from the group consisting of about 1 mg, about 2.5 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, about 2000 mg, about 2050 mg, about 2100 mg, about 2150 mg, about 2200 mg, about 2250 mg, about 2300 mg, about 2350 mg, and about 2400 mg per day. In some embodiments, the dose of tipifarnib is about 150 mg per day. In some embodiments, the dose of tipifarnib is about 300 mg per day. In some embodiments, the dose of tipifarnib is about 450 mg per day. In some embodiments, the dose of tipifarnib is about 600 mg per day. In some embodiments, the dose of tipifarnib is about 900 mg per day. In some embodiments, the dose of tipifarnib is about 1200 mg per day. In some embodiments, the dose of tipifarnib is about 1800 mg per day. In some embodiments, the dose above is administered 1, 2, 3, or 4 times per day. In some embodiments, the dose above is administered once per day. In some embodiments, the dose above is administered twice per day. In some embodiments, the dose of tipifarnib is about 300 mg twice daily (BID). In some embodiments, the dose of tipifarnib is about 600 mg BID. In some embodiments, the dose above is split into two doses that are administered to the subject according to the methods provided herein.

In some embodiments, tipifarnib is administered to the subject according to the methods provided herein at a dose of 0.01-50 mg/kg body weight per day. In some embodiments, the dose of tipifarnib is selected from the group consisting of 0.01-1 mg/kg, 0.01-2.5 mg/kg, 0.01-5 mg/kg, 0.1-5 mg/kg, 0.1-10 mg/kg, 0.1-20 mg/kg, 1-30 mg/kg, 1-40 mg/kg, 5-50 mg/kg, 10-50 mg/kg, 15-50 mg/kg, 20-50 mg/kg, 25-50 mg/kg, 30-50 mg/kg, 40-50 mg/kg, 20-40 mg/kg, and 20-25 mg/kg body weight per day. In some embodiments, the dose of tipifarnib is selected from the group consisting of about 0.01 mg/kg, about 0.02 mg/kg, about 0.05 mg/kg, about 0.06 mg/kg, about 0.07 mg/kg, about 0.08 mg/kg, about 0.09 mg/kg, about 0.1 mg/kg, about 0.5 mg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, and about 50 mg/kg body weight per day. In some embodiments, the per day dose above is split into two amounts, such as two equal amounts, that are administered to the subject according to the methods provided herein. In some embodiments, the per day dose above is administered across 1, 2, 3, or 4 times per day, for example, is administered once or twice per day, such as once per day.

In some embodiments, the dose of tipifarnib is administered to the subject monthly, weekly, or daily, according to the methods provided herein. In some embodiments, the dose of tipifarnib is split into two doses that are administered to the subject according to the methods provided herein. In some embodiments, the dose of tipifarnib is administered to the subject 1, 2, 3 or 4 times per day for one or more cycles, for example, once or twice per day for one or more cycles, such as once per day for one or more cycles. In some embodiments, the dose of tipifarnib is administered to the subject 1, 2, 3 or 4 times per day continuously for unlimited days or until remission achieved in said subject. In some embodiments, tipifarnib is administered to the subject once per day (sometimes referred to as QD) for one or more cycles, such as QD for two or more cycles, QD for three or more cycles, or QD for four or more cycles. In some embodiments, tipifarnib is administered to the subject BID for one or more cycles, such as BID for two or more cycles, BID for three or more cycles, or BID for four or more cycles. In some embodiments, the cycle (sometimes referred to herein as a treating cycle or maintenance cycle) is 1 day, 7 days, 14 days, 21 days, or 28 days. In some embodiments, the 28-day cycle is preferred. In some embodiments, tipifarnib is administered to the subject once per day (QD) for one or more 28-day cycles. In some embodiments, tipifarnib is administered to the subject twice per day (BID) for one or more 28-day cycles. In some embodiments, tipifarnib is administered to the subject once or twice per day every other week during a 28-day cycle, with alternating weeks of rest.

In some embodiments, tipifarnib is administered to the subject 1, 2, 3 or 4 times per day on days 1-7, days 1-7 and 15-21, days 1-21, or each day (i.e., days 1-28) of a 28-day cycle, for one of more cycles, according to the methods provided herein. For example, in some embodiments, tipifarnib is administered to the subject QD on days 1-7, days 1-7 and 15-21, days 1-21, or each day (i.e., days 1-28) of a 28-day cycle, for one of more cycles. For example, in some embodiments, tipifarnib is administered to the subject BID on days 1-7, days 1-7 and 15-21, days 1-21, or each day (i.e., days 1-28) of a 28-day cycle, for one of more cycles. In some embodiments, tipifarnib is administered to the subject on QD on days 1-7 of a 28-day cycle, for one of more cycles. In some embodiments, tipifarnib is administered to the subject BID on days 1-7 of a 28-day cycle, for one of more cycles. In some embodiments, tipifarnib is administered to the subject QD on days 1-7 and 15-21 of a 28-day cycle, for one of more cycles. In some embodiments, tipifarnib is administered to the subject BID on days 1-7 and 15-21 of a 28-day cycle, for one of more cycles. In some embodiments, tipifarnib is administered to the subject QD on days 1-21 of a 28-day cycle, for one of more cycles. In some embodiments, tipifarnib is administered to the subject BID on days 1-21 of a 28-day cycle, for one of more cycles. In some embodiments, tipifarnib is administered to the subject QD on each day (i.e., days 1-28) of a 28-day cycle, for one of more cycles. In some embodiments, tipifarnib is administered to the subject BID on each day (i.e., days 1-28) of a 28-day cycle, for one of more cycles.

In some embodiments, the PI3K inhibitor administered to the subject according to the methods provided herein is alpelisib. In some embodiments, the alpelisib is administered to the subject at a dose of 10-400 mg per day. In some embodiments, the dose of the alpelisib administered to the subject is selected from the group consisting of 10-300 mg, 10-200 mg, 10-150 mg, 10-100 mg, 10-50 mg, 25-300 mg, 25-200 mg, 25-150 mg, 25-100 mg, 25-50 mg, 50-400 mg, 50-300 mg, 50-200 mg, 50-150 mg, 50-100 mg, 100-400 mg, 100-300 mg, 100-200 mg, 150-250 mg, 175-225 mg, 200-400 mg, or 200-300 mg per day. In some embodiments, the alpelisib is administered to the subject at a dose selected from the group consisting of about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 260 mg, about 270 mg, about 275 mg, about 280 mg, about 290 mg, about 300 mg, about 325 mg, about 350 mg, about 375 mg or about 400 mg per day. In some embodiments, the dose of the alpelisib administered to the subject is about 25 mg, about 50 mg, about 100 mg, about 150 mg, about 200 mg, about 250 mg, or about 300 mg per day. In some embodiments, the dose of the alpelisib administered to the subject is about 50 mg, about 150 mg, or about 200 mg per day. In some embodiments, the dose of the alpelisib administered to the subject is about 100 mg, about 150 mg, about 200 mg, about 250 mg or about 300 mg per day. In some embodiments, the alpelisib is administered to the subject at a dose of about 50 mg per day. In some embodiments, the alpelisib is administered to the subject at a dose of about 100 mg per day. In some embodiments, the alpelisib is administered to the subject at a dose of about 150 mg per day. In some embodiments, the alpelisib is administered to the subject at a dose of about 200 mg per day. In some embodiments, the alpelisib is administered to the subject at a dose of about 250 mg per day. In some embodiments, the alpelisib is administered to the subject at a dose of about 300 mg per day. In some embodiments, the dose above of the alpelisib is split into two doses that are administered to the subject according to the methods provided herein. In some embodiments, the alpelisib is administered to the subject 1, 2, 3 or 4 times per day, for example, is administered once or twice per day, such as once per day.

In some embodiments, the per day dose of the alpelisib is administered to the subject daily for one or more cycles according to the methods provided herein. In some embodiments, the per day dose of the alpelisib is split into two doses that are administered to the subject according to the methods provided herein. In some embodiments, the per day dose of the alpelisib is administered across 1, 2, 3 or 4 times per day for one or more cycles, for example, is administered once or twice per day for one or more cycles, such as once per day for one or more cycles. In some embodiments, the alpelisib is administered to the subject 1, 2, 3 or 4 times per day continuously for unlimited days or until remission achieved in said subject. In some embodiments, the alpelisib is administered to the subject once per day (QD) for one or more cycles, such as QD for two or more cycles, QD for three or more cycles, or QD for four or more cycles. In some embodiments, the alpelisib is split into two doses that are administered to the subject according to the methods provided herein. For example, in some embodiments, the alpelisib is administered to the subject twice per day (BID) for one or more cycles, such as BID for two or more cycles, BID for three or more cycles, or BID for four or more cycles. In some embodiments, the cycle (e.g., a treating cycle or maintenance cycle) is 1 day, 7 days, 14 days, 21 days, or 28 days. In some embodiments, the 28-day cycle is preferred. In some embodiments, the alpelisib is administered to the subject once per day for one or more 28-day cycles. In some embodiments, the alpelisib is administered to the subject twice per day for one or more 28-day cycles. In some embodiments, the alpelisib is administered to the subject once or twice per day every other week during a 28-day cycle.

In some embodiments, the methods provided herein comprises (1) an escalating dosing cycle, followed by (2) one or more treating cycles (sometimes referred to as maintenance cycles). In some embodiments, the methods provided herein comprises (1) an escalating dosing cycle, comprising administering (a) escalating daily doses of tipifarnib and (b) a dose of alpelisib, followed by (2) one or more treating cycles (sometimes referred to as maintenance cycles), comprising administering (a) a dose of tipifarnib and (b) a dose of the alpelisib. In some embodiments, tipifarnib is administered 1, 2, 3, or 4 times per day during the escalating dosing cycle, for example, once or twice per day. In some embodiments, the escalating dosing cycle is for 1 day, 2 days, 3 days, 4 days, 5 days, 6 days, 7 days, 14 days, 21 days, or 28 days. In some embodiments, inclusion of an escalating dosing cycle provides a synergistic or therapeutic benefit to the subject, including but not limited to, identifying a therapeutically effective dose of tipifarnib for the subject; identifying a therapeutically effective dose of the alpelisib for the subject; improving the efficacy of the alpelisib; improving the efficacy of cetuximab; mitigating or avoiding toxicities, adverse events or adverse symptoms, or combinations thereof, associated with tipifarnib; mitigating or avoiding toxicities, adverse events or adverse symptoms, or combinations thereof, associated with alpelisib; or mitigating or avoiding toxicities, adverse events or adverse symptoms, or combinations thereof, associated with cetuximab; or combinations thereof.

In some embodiments, the methods provided herein comprises escalating the therapeutically effective amount of the alpelisib administered per day or administered per dose. For example, in some embodiments, the therapeutically effective amount of the alpelisib is escalated from 150 mg to 200 mg, from 150 to 250 mg, from 150 to 300 mg, from 200 mg to 250 mg, from 200 mg to 300 mg, or from 250 mg to 300 mg, once per day. In some embodiments, the therapeutically effective amount of the alpelisib administered per day or administered per dose is escalated while maintaining the therapeutically effective amount of the tipifarnib administered per day or administered per dose.

In some embodiments, the methods provided herein comprises escalating the therapeutically effective amount of the tipifarnib administered per day or administered per dose. For example, in some embodiments, the therapeutically effective amount of the tipifarnib is escalated from 300 mg to 600 mg, 600 mg to 900 mg, 900 mg or 1200 mg, 1200 mg to 1500 mg, 1500 mg to 1800 mg per day, such as from 300 mg to 600 mg, from 600 mg to 900 mg, or from 900 mg to 1200 mg, or up to 1800 mg per day. In some embodiments, such total per day doses of tipifarnib are administered once daily or twice daily. For example, in some embodiments, a daily dose of tipifarnib of 600 mg is administered as 300 mg twice daily (e.g., morning and evening), a daily dose of tipifarnib of 900 mg is administered twice daily (e.g., 600 mg in the morning and 300 mg in the evening, or 300 mg in the morning and 600 mg in the evening), a daily dose of tipifarnib of 1200 mg is administered as 600 mg twice daily (e.g., morning and evening), and a daily dose of tipifarnib of 1800 mg is administered as 900 mg twice daily (e.g., morning and evening). In some embodiments, the therapeutically effective amount of the tipifarnib administered per day or administered per dose is escalated while maintaining the therapeutically effective amount of the alpelisib administered per day or administered per dose.

In some embodiments, the methods provided herein comprises (1) a loading dosing cycle, followed by (2) one or more treating cycles (sometimes referred to as maintenance cycles). In some embodiments, the methods provided herein comprises (1) a loading dosing cycle, comprising administering (a) a loading dose of tipifarnib and (b) a dose of alpelisib, followed by (2) one or more treating cycles (sometimes referred to as maintenance cycles), comprising administering (a) a dose of tipifarnib and (b) a dose of the alpelisib. In some embodiments, the loading dose (sometimes referred to as an elevated dose or a bolus dose) of tipifarnib is 1.1-10 times the dose administered during the one or more treating cycles. For example, in some embodiments, the loading dose of tipifarnib is 1.1, 1.2, 1.3, 1.4, 1.5, 1.6, 1.7, 1.8, 1.9, 2, 3, 4, 5, 6, 7, 8, 9, or 10 times the dose administered during the one or more treating cycles. In some embodiments, administration of tipifarnib during the loading dosing cycle is 1, 2, 3, or 4 times per day. In some embodiments, administration of tipifarnib during the loading dosing cycle is once per day. In some embodiments, administration of tipifarnib during the loading dosing cycle is twice per day. In some embodiments, the loading dosing cycle is for 1 day, 2 days, 3 days, 4 days, 5 days, 6 days, 7 days, 14 days, 21 days, or 28 days. In some embodiments, inclusion of a loading dosing cycle prior to treating the subject with one or more treating or maintenance cycles, provides a synergistic or therapeutic benefit to the subject, including but not limited to, mitigating or avoiding toxicities, adverse events or adverse symptoms, or combinations thereof, associated with tipifarnib; mitigating or avoiding toxicities, adverse events or adverse symptoms, or combinations thereof, associated with alpelisib; or mitigating or avoiding toxicities, adverse events or adverse symptoms, or combinations thereof, associated with cetuximab; or combinations thereof.

In some embodiments, the dose of tipifarnib and the dose of the alpelisib, are administered to the subject concurrently or sequentially. In some embodiments, the dose of tipifarnib is administered to the subject before the administration of the dose of the alpelisib. In some embodiments, the dose of tipifarnib is administered to the subject after the administration of the dose of the alpelisib. In some embodiments, the dose of tipifarnib is administered to the subject QD or BID on days 1-7, days 1-7 and 15-21, days 1-21, or each day, of a 28-day treatment cycle, and the dose of the alpelisib is administered QD or BID each day of the 28-day treatment cycle. For example, in some embodiments, the dose of tipifarnib is administered to the subject QD on days 1-7, days 1-7 and 15-21, days 1-21, or each day, of a 28-day treatment cycle, and the dose of the alpelisib is administered QD each day of the 28-day treatment cycle. In some embodiments, the dose of tipifarnib is administered to the subject BID on days 1-7, days 1-7 and 15-21, days 1-21, or each day, of a 28-day treatment cycle, and the dose of the alpelisib is administered QD each day of the 28-day treatment cycle. In some embodiments, the dose of tipifarnib is administered to the subject QD on days 1-7, days 1-7 and 15-21, days 1-21, or each day, of a 28-day treatment cycle, and the dose of the alpelisib is administered BID each day of the 28-day treatment cycle. In some embodiments, the dose of tipifarnib is administered to the subject BID on days 1-7, days 1-7 and 15-21, days 1-21, or each day, of a 28-day treatment cycle, and the dose of the alpelisib is administered BID each day of the 28-day treatment cycle.

### 7. EXAMPLES

General Methods

Cell lines were obtained from ATCC (SCC25, SCC9, FaDu, and Detroit 562), JCRB (SAS, HSC2, HSC3), DSMZ (CAL33), or Sigma (PECAP15J, BICR22, UMSCC17B) and maintained in a humidified atmosphere with 5% CO₂ at 37° C. Cells were maintained in DMEM (Detroit 562, FaDu, HSC2, HSC3, CAL33, PECAP15J, BICR22) or DMEM/F12 (SCC25, SCC9, SAS) supplemented with 10% FBS and penicillin/streptomycin. All lines tested negative for mycoplasma.

Alpelisib was purchased from MedChemExpress and dissolved in DMSO. Dharmacon ON-TARGETplus siRNA SMARTPools against RHEB and HRAS were obtained from Horizon Discovery and transfected using Lipofectamine RNAiMAX (ThermoFisher).

### Example 1: Tipifarnib and Alpelisib Combination Therapy

Trial Design

A Phase 1/2, open-label, 2 drug dose escalation trial (KO-TIP-013) evaluates the safety of the combination of tipifarnib and alpelisib and determines the recommended combination dosing regimen of the FTI tipifarnib in combination with the PI3Kα inhibitor alpelisib in adult participants with R/M HNSCC who meet criteria for at least one of 2 biomarker-defined cohort:
Tumors harboring PIK3CA (activating) mutations and/or amplifications as determined by next generation sequencing (NGS) on a tumor tissue biopsy (Cohort 1); and
Tumors with increased HRAS dependency based on HRAS overexpression as determined the Sponsor's central laboratory (Cohort 2).

PIK3CA and HRAS status should be determined using tumor tissue obtained at the time of or after the most recent progression; however, tissue obtained prior may be used when this is not possible, and with sufficient justification. If several tissue samples are available, testing should occur on the most recently obtained sample. Each cohort will be conducted in parallel and independent of the other cohort, though safety information for the combination may influence the dosing of both cohorts. Each cohort will be analyzed separately. The Bayesian logistic regression modeling (BLRM) approach will be utilized in the trial to characterize safety, tolerability, and clinical activity of the combination of tipifarnib and alpelisib with the goal of identifying an optimal biologically active combination dose (OBAD) (Neuenschwander et al., Statistical Methods in Drug Combination Studies (2014)). This approach uses joint binary toxicity and efficacy endpoint models for each individual agent (tipifarnib and alpelisib) in conjunction with a combination agent modeling approach to provide dose recommendations for both tipifarnib and alpelisib. This strategy will be used in the 2-drug dose escalation/de-escalation design in order to determine the safest and most effective dosing regimen for each defined cohort, while minimizing the potential for subtherapeutic dosing. Investigator assessment of the toxicity and efficacy for each participant who receives sufficient trial intervention will be used to inform the models, and the dose escalation/de-escalation recommendations of the safety monitoring committee (SMC).

The modeling is based upon the following endpoints:
Endpoint 1: Binary endpoint (Dose Limiting Toxicity (“DLT”)).
Endpoint 2: Binary endpoint (efficacy response based on overall response).
Endpoint 3: Binary endpoint (efficacy response based on SD).

The safe doses will be identified using the dose-toxicity models for Endpoint 1. A combination dose will be considered safe if the posterior probability of excessive toxicity at the combination dose does not exceed a pre-defined threshold of 25%. This approach to characterizing the degree of toxicity is similar to the escalation with overdose control (EWOC) principle (Babb et al., Stat. Med., 17:1103-20 (1998); Rogatko et al., J. Clin. Oncol., 25:4982-6 (2007)) that is commonly applied in dose escalation trials to control the risk of exposing participants to toxic doses.

A clinical utility index will be defined to facilitate the process of identifying safe doses with a desirable efficacy profile for the next cohort of participants. To define the index, the posterior probability of excessive toxicity will be computed based on Endpoint 1 and the posterior probability of target efficacy response will be computed based on Endpoints 2 and 3 for all eligible combination doses. The DLT period for this trial and the initial efficacy period to inform the BLRM will be considered the first 28 days (Cycle 1) following the start of trial intervention.

The BLRM model will only be run for evaluable participants. A participant will be evaluable if he/she has received at least 75% of the planned dose for each trial intervention and completed the first efficacy assessment after Cycle 1 Day 28; however, any event meeting DLT criteria (see Table 2) experienced prior to the completion of the 28 days will be considered a DLT probability of excessive toxicity will be computed based on Endpoint 1 and the posterior probability of target efficacy response will be computed based on Endpoints 2 and 3 for all eligible combination doses. The DLT period for this trial and the initial efficacy period to inform the BLRM will be considered the first 28 days (Cycle 1) following the start of trial intervention.

The BLRM model will only be run for evaluable participants. A participant will be evaluable if he/she has received at least 75% of the planned dose for each trial intervention and completed the first efficacy assessment after Cycle 1 Day 28; however, any event meeting DLT criteria (per the National Cancer Institute (NCI) Common Terminology Criteria for Adverse Events (CTCAE) v5.0) experienced prior to the completion of the 28 days will be considered a DLT.

Combination Dosages

The planned combinations of tipifarnib and alpelisib for this trial are provided in Table 1.

The projected starting dose of this combination is 600 mg/day (300 mg BID (on Days 1-7, 15-22 of 28-day cycles)) of tipifarnib and 200 mg QAM (once per day at morning) of alpelisib (continuous, Days 1-28 of 28-day cycles). The planned dose levels for tipifarnib are generally well tolerated in previous studies and are within the established therapeutic dose range. The maximum dose level for alpelisib that may be tested in this trial is the 300 mg QAM dose level which is currently marketed for combination use.

This study is designed to evaluate the safety, tolerability and preliminary efficacy of tipifarnib in combination with alpelisib in patients having HRAS-overexpressing and/or PIK3CA-mutated and/or -amplified recurrent/metastatic head and neck squamous cell carcinoma (HNSCC). Patients who meet criteria for at least one of 2 biomarker-defined cohorts:

Tumors harboring PIK3CA (activating) mutations and/or amplifications as determined by next generation sequencing (NGS) on a tumor tissue biopsy (Cohort 1); and

Tumors with increased HRAS dependency based on HRAS overexpression as determined by the Sponsor's central laboratory (Cohort 2).

According to the study design, the anticipated dose levels for the combination are defined in Table 1 below.

TABLE 1
Projected Dosing of Tipifarnib (T) and Alpelisib (A)

| | | Alpelisib |
|---|---|---|
| Combinations | Tipifarnib (T) | (A)**** |
| 1 | 1200 mg/day (600 mg BID) | 300 mg QAM |
| 2 | 1200 mg/day (600 mg BID) | 250 mg QAM |
| 3 | 1200 mg/day (600 mg BID) | 200 mg QAM |
| 4 | 1200 mg/day (600 mg BID) | 150 mg QAM |
| 5 | 900 mg/day (600 mg QAM, | 300 mg QAM |
| | 300 mg QPM)*** | |
| 6 | 900 mg/day (600 mg QAM, | 250 mg QAM |
| | 300 mg QPM)*** | |
| 7 | 900 mg/day (600 mg QAM, | 200 mg QAM |
| | 300 mg QPM)*** | |
| 8 | 900 mg/day (600 mg QAM, | 150 mg QAM |
| | 300 mg QPM)*** | |
| 9 | 600 mg/day (300 mg BID)* | 300 mg QAM |
| 10 | 600 mg/day (300 mg BID)* | 250 mg QAM |
| 11 | 600 mg/day (300 mg BID)* | 200 mg QAM |
| 12 | 600 mg/day (300 mg BID)* | 150 mg QAM |
| 13 | 300 mg/day (300 mg QD)** | 300 mg QAM |
| 14 | 300 mg/day (300 mg QD)** | 250 mg QAM |
| 15 | 300 mg/day (300 mg QD)** | 200 mg QAM |
| 16 | 300 mg/day (300 mg QD)** | 150 mg QAM |
| 17 | 1800 mg/day (900 mg BID) | 300 mg QAM |
| 18 | 1800 mg/day (900 mg BID) | 250 mg QAM |
| 19 | 1800 mg/day (900 mg BID) | 200 mg QAM |
| 20 | 1800 mg/day (900 mg BID) | 150 mg QAM |

Abbreviations: BID = twice daily; QAM = each morning; QD = once daily; QPM = each evening; T = tipifarnib.
*Initial Cohort Starting Dose
**Single daily dose should be taken at the same time each day with food.
***900 mg/day should be divided into 600 mg QAM and 300 mg QPM, though reversal of doses (300 mg QAM and 600 mg QPM) is permitted as long as consistently done throughout the cycle. The specifics of dosing should be recorded in the dosing diary
****Single daily dose should be taken orally at approximately the same time each day with food (preferably in the morning).

Dose escalations or reductions of either tipifarnib or alpelisib can be made in a stepwise fashion, by increasing or decreasing the dose of one of the two agents while maintaining the dose amount of the other agent. For example, a dose escalation of alpelisib from the above noted projected starting dose amounts of this combination (combination #11 in Table 1) would be going from combination #11 to #10 in Table 1, while a dose reduction of alpelisib from the above noted projected starting dose amounts of this combination would be going from combination #11 to #12 in Table 1. For example, a dose escalation of tipifarnib from the above noted projected starting dose amounts of this combination (combination #11 in Table 1) would be going from combination #11 to #7, or going from combination #11 to #7 and then from combination #7 to #3, in Table 1, while a dose reduction of tipifarnib from the above noted projected starting dose amounts of this combination would be going from combination #11 to #15 in Table 1.

TABLE 2
Dose Limiting Toxicity (DLT)

| TOXICITY | DLT CRITERIA |
|---|---|
| Hematologic | CTCAE Grade 4 neutropenia for more than 7 consecutive days |
| | CTCAE Grade 4 thrombocytopenia |
| | CTCAE Grade 3 thrombocytopenia with bleeding |
| | Febrile neutropenia (decrease in neutrophils associated with fever ≥38.5° C., |
| | Absolute neutrophil count (ANC) < 1.0 × 10⁹/L) |
| Metabolism and nutrition | Any CTCAE grade hyperglycemia leading to diabetic keto-acidosis, hospitalization |
| disorders | for intravenous insulin infusion, or non-ketotic coma |
| | FG > 250-500 mg/dL (confirmed with a repeat FPG within 24 hours) that does not |
| | resolve to ≤250 mg/dL within 7 days (after initiation of oral antidiabetic treatment) |
| | FG > 160-250 mg/dL (confirmed with a repeat FPG within 24 hours) that does not |
| | resolve to ≤160 mg/dL within 21 consecutive days (after initiation of oral |
| | antidiabetic treatment) |
| | Grade 3 weight loss that does not improve/resolve within 14 days with optimal |
| | medical management including nutritional intervention |
| Hepatic disorders | CTCAE Grade 2 bilirubin for more than 7 consecutive days |
| | ≥CTCAE Grade 3 total bilirubin |
| | ≥CTCAE Grade 2 ALT in conjunction with blood bilirubin Grade ≥2 of any |
| | duration in the absence of liver metastases |
| | ≥CTCAE Grade 3 ALT for more than 4 consecutive days |
| | ≥CTCAE Grade 4 ALT or AST |
| Gastrointestinal disorders | Diarrhea CTCAE Grade ≥3 for 48 hrs or more despite optimal anti-diarrhea |
| | treatment |
| | CTCAE Grade ≥3 nausea that does not improve to < Grade 3 in ≤7 days with |
| | optimal medical management |
| | Grade 3 vomiting that does not improve to < Grade 3 in ≤7 days with optimal |
| | medical management |
| | Grade 3 stomatitis that does not improve to < Grade 3 in ≤7 days with optimal |
| | medical management |
| Cardiac disorders | Cardiac toxicity ≥ CTCAE Grade 3 |
| Pulmonary disorders | Pulmonary toxicity ≥ CTCAE Grade 3 as defined by MedDRA SMQ narrow |
| | “Interstitial Lung Disease” |
| Rash | CTCAE Grade 3 rash for more than 7 days despite optimal treatment including |
| | systemic steroid use |
| | CTCAE Grade 4 rash |
| Renal | Grade 3 renal toxicity that cannot be corrected to < Grade 3 with best supportive |
| | care within 7 days |
| Non-hematologic | Any grade 4 non hematologic toxicity, except for the exclusions noted below in the |
| | “Exception for DLT criteria” field. |
| | >5 days of CTCAE Grade 3 fatigue |
| | Any grade 3 non-hematologic toxicity with >1 week of duration excluding: |
| | Alopecia |
| | Asthenia |
| | Insomnia |
| Exception for DLT criteria | Grade ≥3 laboratory abnormalities that are responsive to oral |
| | supplementation or deemed by the Investigator to be clinically |
| | insignificant |
| | Grade 3 headache that improves to < Grade 3 in ≤7 days with optimal |
| | medical management |
| | Grade 3 Loss of appetite/anorexia |
| | Grade ≥3 laboratory abnormalities that are responsive to oral |
| | supplementation or deemed by the Investigator to be clinically |
| | insignificant |

Discontinuation of either combination agent and continuation on trial with a single agent is not permitted.

### Example 2: Study Population Inclusion Criteria and Exclusion Criteria

Patients are eligible to be included in the study only if all of the following criteria apply.

Inclusion Criteria

For inclusion of a participant in the trial, all the following inclusion criteria must be fulfilled. If a participant does initially not meet any inclusion criteria, the participant may be re-screened at a later time.
1. At least 18 years of age.
2. Histologically confirmed head and neck cancer (oral cavity, pharynx, larynx, sinonasal, nasopharyngeal, or unknown primary) of squamous histology not amenable to local therapy with curative intent (surgery or radiation therapy with or without chemotherapy).
3. Documented treatment failure from at least 1 prior therapy (e.g., tumor progression, clinical deterioration, or recurrence) in the R/M setting, with the following exception:
   Participant is considered not appropriate for treatment with standard of care in this setting, with documented rationale.
There is no limit on the number of prior lines of therapy a participant may have received.
4. Measurable disease by Response Evaluation Criteria in Solid Tumors (RECIST) v1.1 that meets the criteria for selection as a target lesion according to RECIST v1.1. The presence of at least one measurable target lesion per RECIST v1.1 must be confirmed by local radiology prior to participant entry.
5. Has a tumor that per criteria outlined in the protocol, is dependent upon HRAS and/or PIK3CA.
6. At least 2 weeks or 5 half-lives, whichever is shorter, since the last systemic therapy regimen prior to Cycle 1 Day 1, with the following exception:
   Last dose of any prior checkpoint inhibitor therapy must have been administered at least 2 weeks prior to Cycle 1 Day 1.
   Participants must have recovered to NCI CTCAE v5.0<Grade 2 from all acute toxicities (excluding Grade 2 toxicities that are not considered a safety risk by the Sponsor and Investigator), or toxicity must be deemed irreversible by the Investigator.
7. At least 2 weeks since last radiotherapy to the time of first dose.
   Participants must have recovered from all acute toxicities from radiotherapy.
8. Eastern Cooperative Oncology Group (ECOG) performance status of 0-1.
9. Acceptable liver function:
   a) Bilirubin ≤1.5 times upper limit of normal (×ULN).
   b) Aspartate aminotransferase (AST) (serum glutamic oxaloacetic transaminase [SGOT]) and alanine aminotransferase (ALT) (serum glutamic pyruvic transaminase [SGPT])
      ≤1.5×ULN.
   The participant must meet/continue to meet these criteria at the time of first dosing, as confirmed by analysis within 72 hours of Cycle 1 Day 1.
10. Acceptable renal function with either serum creatinine ≤1.5×ULN or a calculated creatinine clearance ≥60 mL/min using the Cockcroft-Gault or Modification of Diet in Renal Disease formulas.
   The participant must meet/continue to meet these criteria at the time of first dosing, as confirmed by review of analysis performed within 72 hours of Cycle 1 Day 1.
11. Acceptable hematologic status:
   a) Absolute neutrophil count (ANC)≥1000 cells/μL.
   b) Platelet count ≥75,000/μL.
   c) Hemoglobin ≥8.0 g/dL.
      The participant must meet/continue to meet these criteria at the time of first dosing, as confirmed by review of analysis performed within 72 hours of Cycle 1 Day 1.
12. Participants who have Type 2 diabetes mellitus must meet criteria for acceptable diabetes control, which includes fasting glucose ≤140 mg/dL (7.7 mmol/L) and HbA1c <6.4% at the time of enrolment.
13. Fasting serum amylase ≤2×ULN.
14. Fasting serum lipase ≤ULN.
15. Must be able to take oral medications with food. Must be able to swallow alpelisib whole tablet or oral suspension containing crushed tablets.
   Note: Feeding tube may not be used for alpelisib administration.
16. Female participants of child-bearing potential and male participants with female partners of child-bearing potential must follow protocol-specified guidance for avoiding pregnancy while on treatment and for and at least 28 days after last dose of tipifarnib for females and 90 days for males. Female participants must have a negative serum pregnancy test within 72 hours prior to start of trial intervention.
17. Capable of giving signed informed consent, which includes compliance with the requirements and restrictions listed in the informed consent form (ICF) and in this protocol.

Exclusion Criteria

Participants are excluded from the trial if any of the following criteria apply. If a participant initially meets any exclusion criteria, the participant may be re-screened at a later time.
1. Has disease that is suitable for therapy administered with curative intent.
2. Histologically confirmed salivary gland, thyroid, (primary) cutaneous squamous or non-squamous histologies (eg, mucosal melanoma).
3. Ongoing treatment with an anticancer agent (excluding adjuvant hormonal therapy for breast cancer and hormonal treatment for castration sensitive prostate cancer) or any known additional malignancy that is progressing or requires active treatment (excluding non-melanoma skin cancer, adjuvant hormonal therapy for breast cancer and hormonal treatment for castration sensitive prostate cancer).
4. Prior treatment (at least 1 full treatment cycle) with an FTI.
5. Prior treatment (at least 1 full treatment cycle) with a PI3K, mTOR, or AKT inhibitor.
6. Any use of investigational therapy within 2 weeks of Cycle 1 Day 1 or 5 half-lives (whichever is longer).
   Last dose of any prior checkpoint inhibitor therapy must have been administered at least 2 weeks prior to Cycle 1 Day 1.
7. Received treatment for unstable angina, myocardial infarction, and/or cerebro-vascular attack within the prior 6 months.
   A history of New York Heart Association Grade III or greater congestive heart failure.
   Current serious cardiac arrhythmia requiring medication, except atrial fibrillation.
8. Non-tolerable Grade 2, or ≥Grade 3 neuropathy or evidence of unstable neurological symptoms within 4 weeks of Cycle 1 Day 1.
   Non-tolerable Grade 2 toxicities are defined as those with moderate symptoms that the participant is not able to endure for the conduct of instrumental activities of daily life or that persists ≥7 days.
9. Mean corrected QT interval by Fredericia's formula (QTcF) >480 ms on triplicate electrocardiograms (ECGs) performed within 5 minutes of each other.
10. Major surgery, other than diagnostic surgery, within 2 weeks prior to Cycle 1 Day 1, without complete recovery.
11. Active, uncontrolled bacterial, viral, or fungal infections requiring systemic therapy.
12. Participant with an established diagnosis of diabetes mellitus Type 1 or not controlled Type 2 (based on fasting glucose and HbA1c).
13. Participant has impairment of gastrointestinal (GI) function or GI disease that may significantly alter the absorption of the trial drugs (eg, ulcerative diseases, uncontrolled nausea, vomiting, diarrhea, malabsorption syndrome, or small bowel resection) based on Investigator discretion.
14. Participant has currently documented pneumonitis/interstitial lung disease.
15. Participant has a history of acute pancreatitis within 1 year of screening or past medical history of chronic pancreatitis.
16. Participant with unresolved osteonecrosis of the jaw.
17. Participant has a history of severe cutaneous reaction, such as Stevens-Johnson Syndrome (SJS), Erythema Multiforme (EM), Toxic Epidermal Necrolysis (TEN), or Drug Reaction with Eosinophilia and Systemic Symptoms (DRESS).
18. Participant who has history of allergic reactions to tipifarnib (or structural compounds similar to tipifarnib or to its excipients) or alpelisib (or structural compounds similar to alpelisib or to its excipients).
   This includes hypersensitivity to imidazoles, such as clotrimazole, ketoconazole, miconazole, and others in this drug class.
19. Participant is currently receiving or has received systemic corticosteroids ≤2 weeks prior to starting trial drug, or who have not fully recovered from side effects of such treatment. Note: The following uses of corticosteroids are permitted: single doses, topical applications (eg, for rash), inhaled sprays (eg, for obstructive airways diseases), eye drops or local injections (eg, intra-articular).
20. Required use of concomitant medications classified as the following that cannot be discontinued at least 7 days prior to the start of treatment:
   strong inhibitors or inducers of cytochrome P450 3A4 (CYP3A4,
   strong inhibitors or inducers uridine 5′-diphospho-glucuronosyltransferase (UDP)-glucuronosyltransferase (UGT).
   Inhibitors of BCRP
21. Concomitant disease or condition that could interfere with the conduct of the trial or that would, in the opinion of the Investigator, pose an unacceptable risk to the participant in this trial.
22. Female participant who is pregnant or lactating.
23. Unwillingness or inability to comply with the trial protocol for any reason.

### Example 3: Study Assessments and Procedures

Efficacy Assessments

The presence of at least one measurable target lesion per RECIST v1.1 must be confirmed by local radiology prior to enrollment. Participants without at least one measurable target lesion confirmed by local radiology will not be enrolled in the trial. Note: Confirmation of RECIST v1.1 measurable disease by local radiology is required for trial eligibility.

Objective response (CR and PR) as determined by the participant's best tumor response, DOR, and time to progression will be assessed using RECIST v1.1 (Appendix 3) by Investigator assessment. Confirmation of response is required according to RECIST v1.1 guidelines. Tumor response assessments will continue until disease progression, initiation of new anticancer therapy, or trial withdrawal.

Method and Timing

Radiological assessments of the tumor lesions will be made at screening (within 4 weeks prior to first study intervention administration on Cycle 1 Day 1, though if not possible scans must have been performed within 60 days of Cycle 1 Day 1), at least once approximately every 8 weeks (f 5 days) for the remainder of the first 12 months of study intervention (through and including Cycle 13), and once approximately every 12 weeks (f 5 days) for year 2 and beyond of study intervention. Additional tumor assessments may be conducted at the judgment of the Investigator but will not substitute for protocol-required assessments. Optional photographs of lesions to coincide with tumor assessments are recommended.

Radiological assessments will be discontinued at the time of trial intervention discontinuation, and completion of End of Treatment procedures (eg, completion of assessments within 4, 8, or 12 weeks before treatment discontinuation or confirmation of response required).

Lesions to be included in the tumor assessments should follow RECIST v1.1. Computed tomography scan with a contrast agent is the preferred imaging method and the same technique should be used at screening and post-treatment assessments. CT scan coverage at screening should encompass scans of the neck (including the skull base), chest and abdomen (including the liver and adrenals). Any other areas of disease involvement should be scanned based on the participant's signs and symptoms.

Participants with contrast allergy or renal insufficiency may use non-contrast CT or magnetic resonance imaging (MRI), whichever is required to adequately assess all disease. For participants who develop a contraindication to contrast after screening scans are performed with a contrast agent, the decision to use non-contrast CT or MRI (enhanced or non-enhanced) should be based on tumor type, anatomic location of disease and should be optimized to allow for comparison to the prior scans if possible. The one exception where MRI would not be recommended is for the evaluation of parenchymal lung metastases. In this instance, CT would be preferred. If imaging of the brain is indicated, MRI of the brain with and without gadolinium should be performed for optimal evaluation of the brain. If MRI is medically contraindicated, CT of the brain with and without contrast would be suggested.

Survival Follow-Up

Beginning at the time of the End of Treatment visit, follow-up contact with the participant and/or caregiver(s) (eg, electronic technology-based, telephone, or in person) is to occur approximately every 12 weeks (f 1 week) for survival and the use of subsequent therapy until either death or End of Trial. Information on survival and subsequent anticancer therapy for HNSCC will be collected including name, dates (start, end), treatment outcome (response and response criteria), DOR, and date of progression. Information will also be collected on subsequent cancer surgery for HNSCC including date of surgery. Information will be collected on subsequent radiological treatments for HNSCC including anatomical site(s) and date(s) of radiation treatment.

Pharmacokinetics

The blood samples for PK analysis will be collected from patients at the time points described in Table 3.

TABLE 3
Blood Sampling Timepoints for PK Analysis

| | Blood Sample Collection |
|---|---|
| Blood Sampling Day | Time Window |
| Cycle 1 Day 1 | Pre-dose, 0.5, 1, 2, 3, 4, 6, 8, 12 |
| | and 24 hours after dosing |
| Cycle 2 Day 1 | Pre-dose, 0.5, 1, 2, 3, 4, 6, 8, 12 |
| | and 24 hours after dosing |
| Tumor Response Assessment visits¹ | Anytime during the visit |
| Cycle 3 Day 1 | Anytime during the visit |
| Cycle 4 Day 1 | Anytime during the visit |
| Cycle 5 Day 1 | Anytime during the visit |
| Cycle 6 Day 1 | Anytime during the visit |

Abbreviations: PK = pharmacokinetics
¹If the tumor response assessment visit coincides with a Day 1 visit where a blood sample for PK is also scheduled for collection, follow the PK timepoints defined for the respective Day 1 visit.
Window:
Pre-dose: within 15 minutes prior to dose 30 minute sample: ±5 minutes
1-4 hours: ±10 minutes
6-12 hours: ±30 minutes
12 and 24 hours (but prior to next dose): ±2 hours

The time and date of blood sample collection will be collected and recorded in the eCRF. Additionally, on study visit days when a blood sample for PK analysis is collected, the time, date and dosage of the patient's last dose of tipifarnib and alpelisib (most recent dose to the PK blood sample) will be collected and recorded in the eCRF.

Up to 3 additional blood samples for the determination of tipifarnib and/or alpelisib plasma concentration may be collected at the discretion of the SMC and/or Sponsor based on review of ongoing PK and safety data. An additional PK sample may be drawn at Sponsor or Investigator discretion as part of the evaluation of specific AEs.

Biomarkers

Due to limited understanding of the biological activities induced by combining tipifarnib and alpelisib in patients who have cancer, in addition to determining the MTD, the trial will serve to evaluate patterns of biomarkers of response and resistance. To complete these exploratory objectives, enrolled participants will be required to submit archival tumor tissue obtained at the time of, or after, the most recent progression event prior to treatment (baseline tumor tissue) and blood for circulating tumor DNA (ctDNA) (at baseline and on-treatment). Samples collected at baseline may be used to develop diagnostic tests to identify patients who will benefit most from a new drug, also known as companion diagnostics. Optional tumor tissue will be collected at tumor assessments and at the end of treatment (if available and aligned with routine clinical care at the time of the assessment) to investigate potential mechanisms of response and resistance. Analysis of baseline and on-treatment samples will be used to investigate potential prognostic and/or predictive biomarkers of response to tipifarnib.

This will be performed using a combination of genomic, transcriptomic and proteomic technology, which may include profiling of mutation, amplification and/or other somatic gene alteration in DNA, RNA, or protein levels in tumor tissue. Analysis of germline DNA is not planned for this study.

### Example 4: HRAS and PIK3CA Dysregulated HNSCC PDX Models Respond to Combination Treatment

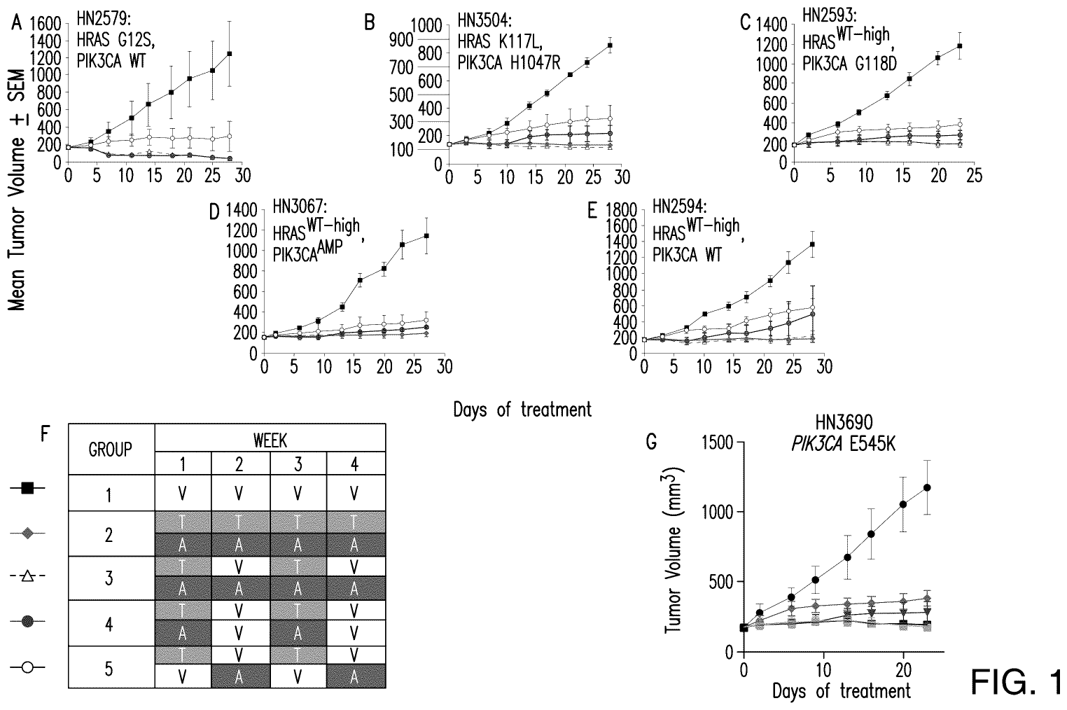

The ability of the combination of tipifarnib and alpelisib to inhibit tumor growth was determined using selected patient-derived xenograft (PDX) models of HNSCC. The selected models expressed different levels or mutations of HRAS and/or different expression levels or mutations of PIK3CA. Female BALB/c nude or nu/nu mice (6-8 weeks) were inoculated subcutaneously on the right flank with primary human tumor model fragment (2-3 mm in diameter) for tumor development. The PDX HNSCC models used were: HN2579 model (FIG. 1A; HRAS mutant), wherein the HRAS mutation is or comprises a modification in a codon that encodes an amino acid substitution at the specified position G12S in the corresponding mutant HRas protein; HN3504 model (FIG. 1B; HRAS mutant and PIK3CA mutant), wherein the HRAS mutation is or comprises a modification in a codon that encodes an amino acid substitution at the specified position K117 in the corresponding mutant HRas protein (here the mutant HRas protein has a K117L mutation), and the PIK3CA mutation is or comprises a modification in a codon that encodes an amino acid substitution at the specified position H1047 in the corresponding mutant PI3K protein (here the mutant PI3K protein has a H1047R mutation); HN2593 model (FIG. 1C; high wild-type HRAS expression levels (referred to as HRAS^{WT-high}) and PIK3CA mutant), wherein the PIK3CA mutation is or comprises a modification in a codon that encodes an amino acid substitution at the specified position G118 in the corresponding mutant PI3K protein (here the mutant PI3K protein has a G118D mutation); HN3067 model (FIG. 1D; high wild-type HRAS expression levels (HRAS^{WT-high}) and PIK3CA amplified (referred to as PIK3CA^{AMP})); HN2594 model (FIG. 1E; high wild-type HRAS expression levels (HRAS^{WT-high}) and having wild-type PIK3CA expression); HN3690 model (FIG. 1G; PIK3CA mutant, HRAS^{WT-high}), wherein the PIK3CA mutation is or comprises a modification in a codon that encodes an amino acid substitution at the specified position E545 in the corresponding mutant PI3K protein (here the mutant PI3K protein has a E545K mutation).

When average tumor size reached about 250-350 mm³, mice were randomly grouped into five (5) dosing regimen groups (FIG. 1F, and FIGS. 1A-1E and 1G), and dosed with tipifarnib (“T”),alpelisib (“A”), and/or with vehicle (20% w/v Hydroxypropyl-o-cyclodextrin), as detailed below and as depicted in FIG. 1F. Specifically, in group 1, the mice were dosed with vehicle (20% w/v Hydroxypropyl-o-cyclodextrin); in group 2, the mice were dosed with tipifarnib (at 60 mg/kg BID) continuously for 4 weeks and alpelisib (at 40 mg/kg QD) continuously for 4 weeks; in group 3, the mice were dosed with tipifarnib (at a dosing of 80 mg/kg BID) at week 1 and 3 of a 4 weeks cycle, with vehicle at weeks 2 and 4 of the 4 weeks cycle, and alpelisib (at 40 mg/kg QD) continuously for 4 weeks; in group 4, the mice were dosed with tipifarnib (at a dosing of 80 mg/kg BID) at week 1 and 3 of a 4 weeks cycle, with vehicle at weeks 2 and 4 of the 4 weeks cycle, and alpelisib (at a dose of 50 mg/kg QD) at week 1 and 3 of the 4 weeks cycle, with vehicle at weeks 2 and 4 of the 4 weeks cycle (i.e. intermittent treatment of the combination of tipifarnib and alpelisib); in group 5, the mice were dosed with tipifarnib (at a dosing of 80 mg/kg BID) at week 1 and 3 of a 4 weeks cycle, with vehicle at weeks 2 and 4 of the 4 weeks cycle, and alpelisib (at a dose of 50 mg/kg QD) at week 2 and 4 of the 4 weeks cycle (i.e. intermittent treatment of the combination of tipifarnib and alpelisib). For each group, tumor dimensions in the mice were measured three times per week. The doses used for the HN3690 PDX models (FIG. 1G) were as above: alpelisib, 40 mg/kg QD for continuous dosing and 50 mg/kg for intermittent dosing; tipifarnib, 60 mg/kg BID for continuous dosing and 80 mg/kg BID for intermittent dosing.

Statistical analysis. To compare tumor volumes of different groups at a pre-specified day, first was used Bartlett's test to check the assumption of homogeneity of variance across all groups. When the p-value of Bartlett's test is ≥0.05, one-way ANOVA was ran to test overall equality of means across all groups. If the p-value of the one-way ANOVA is <0.05, it was further performed post hoc testing by running Tukey's HSD (honest significant difference) tests for all pairwise comparisons, and Dunnett's tests for comparing each treatment group with the vehicle group. When the p-value of Bartlett's test was <0.05, Kruskal-Wallis test was ran to test overall equality of medians among all groups. If the p-value the Kruskal-Wallis test was <0.05, it was further performed post hoc testing by running Conover's non-parametric test for all pairwise comparisons or for comparing each treatment group with the vehicle group, both with single-step p-value adjustment. All statistical analyses were done in R-a language and environment for statistical computing and graphics (version 3.3.1). All tests were two-sided unless otherwise specified, and p-values of <0.05 were regarded as statistically significant. Data in FIGS. 1A-1E and 1G represent means+/−SEM, n=5 mice per group.

For survival analysis, the survival time was analyzed by the Kaplan-Meier method. The survival time was defined as the time from the day of randomization until animal death or ethical endpoint. For each group, the median survival time (MST) and the increased in life-span (ILS) were calculated. The Kaplan-Meier curves were also constructed for each group and the log-rank test was used to compare survival curves between groups.

The PDX models show that dosing regimens that include synchronous dosing of administering of both agents (tipifarnib and alpelisib) most effectively reduces tumor growth in HRAS and PIK3CA dysregulated HNSCC (see e.g., groups 2-4, particularly groups 2-3, in FIGS. 1A-1E and 1G), and appears to be superior to intermittent treatment (wherein both agents are not administered on the same day; e.g., group 5). While continuous dosing of tipifarnib and alpelisib was effective in blocking tumor growth, schedule 3 demonstrated similar tumor growth inhibition and durability of response. Dosing both agents every other week (schedule 4) slightly reduced the activity of the combination. Notably, non-synchronous intermittent dosing (schedule 5) was markedly less effective. (Although synchronous and non-synchronous intermittent dosing schedules appear similarly effective in the HRAS-high PDX model (FIG. 1G), this was due to a single outlier in the synchronous group, and median results favored the synchronous group in that model.)

### Example 5: Tipifarnib Potentiates the Antitumor Effects of PI3Kα Blockade in HNSCC Via Convergent Inhibition of mTOR Activity

The PI3K-AKT-mTOR signaling cascade is the most frequently activated pathway in squamous cell carcinoma of the head and neck (HNSCC). PIK3CA (encoding the a isoform of PI3K's catalytic subunit) is activated by gain-of-function mutation or amplification in approximately 30% of HNSCCs, making PI3Kα an attractive therapeutic candidate. While the PI3Kα inhibitor alpelisib has shown some promise in HNSCC in a phase I setting, its single agent efficacy will likely be limited by feedback re-activation of PI3K or compensatory parallel pathways, necessitating the development of rational combination strategies. Here, we utilize cell line and patient-derived xenograft (PDX) models to evaluate the therapeutic potential of tipifarnib in combination with alpelisib in the PI3K-dysregulated subset of HNSCC.

Because a farnesyltransferase inhibitor may block hyperactivated growth factor signaling at multiple nodes, including HRas protein and Rheb protein, the impact of tipifarnib on the growth of PIK3CA-altered HNSCC models and HRAS-mutant HNSCC models in vitro and in vivo was examined. For tumor spheroid growth assays, cells were resuspended in 4% Matrigel and seeded in 96-well ultralow attachment plates at a density of 1-1.5K cells/well. The following day, spheroids were treated with alpelisib and/or tipifarnib and baseline growth measured using 3D Cell Titer Glo reagent (Promega). Spheroids were incubated with drug for 7 days and a final CTG reading taken. Percentage growth was calculated by[(Ti−Tz)/(C−Tz)]×100 for concentrations for which Ti>/=Tz and [(Ti−Tz)/Tz]×100 for concentrations for which Ti<Tz, where Tz=time zero, C=control growth, and Ti=test growth at each drug concentration. In cell lines harboring PIK3CA mutation or amplification, tipifarnib reduced proliferation of both monolayer and spheroid cultures, and when combined with alpelisib, induced cytotoxicity. Consistently, in PIK3CA mutant/amplified PDX models, the tipifarnib-alpelisib doublet led to deeper antitumor responses compared to alpelisib monotherapy. Simultaneous administration of tipifarnib and alpelisib was superior to split intermittent dosing, hinting at cooperativity between the mechanistic targets of these drugs.

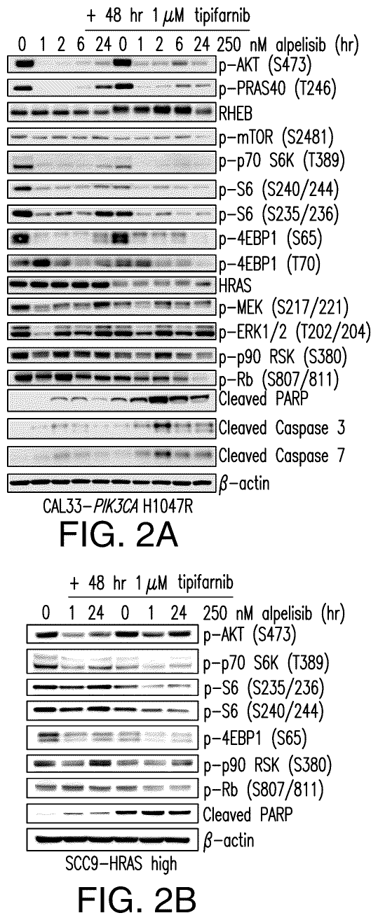

To interrogate the mechanistic underpinnings of this synergy, HNSCC cell lines were exposed to tipifarnib, alpelisib, or the combination, and their effect on RAS/PI3K pathway activity was assessed. In PIK3CA altered lines, single agent tipifarnib or alpelisib reduced phosphorylation of p90 RSK, and mTOR substrates, particularly S6 kinase and ribosomal protein S6. Combination treatment effects were robust and induced rapid apoptosis. In cells exposed to tipifarnib alone, marked rebound of RSK and mTOR substrate phosphorylation occurred after 24 hours, correlating with restored AKT activity. In contrast, though AKT activity rebounded in cells treated with the combination, RSK phosphorylation and markers of mTOR activity (including 4EBP1) remained suppressed (FIG. 2A). Thus, tipifarnib blunts both MAPK and mTOR reactivation following PI3K inhibition. This dual effect implies that the efficacy of tipifarnib in this context stems from inhibition of multiple targets, likely HRas protein and Rheb protein, which converge upon mTOR and synergize with alpelisib to durably block tumor growth.

Immunoblots of MAPK/PI3K pathway components and apoptotic markers in PIK3CA-mutant CAL33 cells treated with alpelisib (BYL-719) for 0, 1, 2, 6, and 24 hours in the absence or presence of tipifarnib (48-hour treatment) are shown in FIG. 2A. A shift in Rheb and HRas protein mobility is indicative of defarnesylation. Generally, immunoblotting was performed as follows: Cell lysates were prepared on ice by washing cells once with PBS, resuspending in 1× cell lysis buffer (Cell Signaling Technology #9803) or RIPA buffer supplemented with Halt protease inhibitor cocktail (Thermo Scientific #78430) and briefly sonicated or vortexed. Lysates were cleared by centrifugation (maximum speed, 10 min) and protein concentration was determined by BCA assay (Pierce). Lysate (20-60 μg) was loaded onto 4-12% Bis-Tris gels (NuPAGE, Invitrogen) for electrophoresis and immunoblotting. When RAS was evaluated, active RAS was detected using Thermo Scientific kit #16117. Membrane and cytosolic fractions were isolated using the Mem-PER Plus Membrane Protein Extraction Kit (Thermo Scientific kit #89842).

In PIK3CA H1047R mutant CAL33 cells, alpelisib rapidly inhibited the activity of AKT, mTOR, and ERK, as indicated by dephosphorylation of their substrates PRAS40, S6K, and RSK, respectively, after 1 hour of treatment (FIG. 2A). However, by 24 hours, phosphorylation of these proteins partially or completely rebounded. In contrast, when cells were treated with tipifarnib (causing HRAS and RHEB to become defarnesylated, as indicated by mobility shift) prior to addition of alpelisib, basal phosphorylation of S6K, S6, and RSK was reduced, the initial inhibition by alpelisib was deeper, and the subsequent rebound was blocked. This potent and durable inhibition of mTOR and RSK by the tipifarnib-alpelisib combination corresponded with cell cycle arrest (dephosphorylation of Rb) and induction of apoptosis (PARP, caspase 3, and caspase 7 cleavage). In sum, combination treatment resulted in stronger inhibition of mTOR activity/target phosphorylation (p70 S6K, S6, 4EBP1), cell cycle arrest (RB phosphorylation), and cell death (PARP and caspase cleavage).

Immunoblots of MAPK/PI3K pathway components and apoptotic markers in high HRas protein (i.e., overexpression of wild-type HRas protein) HNSCC cell line SCC9 cells and in PIK3CA copy gain (amplified) BICR22 cells are shown in FIG. 2B and FIG. 2D, respectively. SCC9 or BICR22 cells were treated with alpelisib (BYL-719) for 0, 1 and 24 hours in the absence or presence of tipifarnib (48-hour treatment). Phosphorylation of S6K, S6, 4EBP1, and RSK was transiently reduced by alpelisib, but rebounded by 24 hours. Combination treatment in each case resulted in stronger inhibition of mTOR activity/target phosphorylation (p70 S6K, S6, 4EBP1), cell cycle arrest (RB phosphorylation), and cell death (cleaved PARP). Addition of tipifarnib reduced baseline phosphorylation of these proteins, deepened their inhibition by alpelisib, and blunted their rebound after 24 hours of alpelisib exposure. More potent TOR and RSK blockade correlated with diminished Rb phosphorylation and induction of PARP cleavage, indicating that the combination inhibited cell cycling and induced cell death, as in PIK3CA-mutant cells.

The effect of the two agents on eIF4F complex formation was investigated. mTORC1 phosphorylates 4EBP1, preventing its binding to eIF4E, allowing eIF4E:eIF4G interaction and initiation of translation. eIF4G (or IgG control) was immunoprecipitated from CAL33 cells treated with DMSO, 250 nM alpelisib (24 hours), 1 μM tipifarnib (48 hours), or the combination. The levels of eIF4G and eIF4E co-immunoprecipitated and in the input cell lysate were assessed using immunoblots. The interaction of eIF4E with eIF4G was markedly reduced by combination treatment, as measured by eIF4G immunoprecipitation, indicating that tipifarnib and alpelisib inhibit protein translation by targeting mTOR activity. Taken together, these findings demonstrate that addition of tipifarnib increases the depth and duration of mTOR inhibition compared to alpelisib alone, leading to cell death in PIK3CA/HRAS-dysregulated HNSCC.

Live cell imaging experiments corroborated the results described above. Combination of tipifarnib and alpelisib induced cytotoxicity in PIK3CA-mutant CAL33 cell line compared to single agents or vehicle, as shown in FIG. 2C. Cytotoxicity was measured using a dye to evaluate the loss of membrane integrity on Incucyte live cell imager. Late apoptosis/necrosis (loss of membrane integrity, nuclei exposure) over time in CAL33 cells treated with alpelisib or tipifarnib monotherapy or the combination for 72 hours measured via Incucyte live cell imaging. Data are means+/−SD of three biological replicates.

Such a dual effect may imply that the efficacy of tipifarnib in this context stems from inhibition of multiple targets, likely HRas protein and Rheb protein, which converge upon mTOR and synergize with alpelisib to durably block tumor growth. Therefore, this combination may provide therapeutic potential for treatment of recurrent/metastatic HNSCCs harboring alterations in PIK3CA.

### Example 6: Tipifarnib and Alpelisib Synergize in PIK3CA-Altered Cell Lines

Sensitivity of a panel of PIK3CA- or HRAS-dysregulated HNSCC cell lines to tipifarnib and alpelisib was assessed. Cell lines with greater than 2.5 copies of PIK3CA were defined as copy gain and a cell line with a high level of active (GTP-bound) HRAS was selected as the HRAS-high model. The cells were cultured as 3D tumor spheroids and treated with increasing concentrations of tipifarnib and/or alpelisib for 7 days as described above in a checkerboard dose-response fashion. Degrees of growth inhibition or cytotoxicity induced by the compounds were assessed and data was collected for 3 biological replicates. Synergy was assessed by Loewe synergy score and sensitivity by a combination sensitivity score. Calculations were made using SynergyFinder R package (Zheng, et al., Genomics Proteomics Bioinformatics, 2022). As shown in Table 4 below, cell lines with gain-of-function PIK3CA mutations, PIK3CA copy gain, or high HRAS activity were sensitive to combined tipifarnib and alpelisib. Additive or synergistic inhibitory effects on cellular viability were observed in these lines with induction or enhancement of cytotoxicity by the combination. In contrast, cell lines lacking both PIK3CA mutation/amplification and HRAS overexpression did not respond to either single agent or to the combination. These results demonstrate that tipifarnib potentiates the antiproliferative effects of alpelisib in in vitro models of PI3Kα/HRAS-dysregulated HNSCC.

TABLE 4

| | PIK3CA | HRAS | HRAS | Combination | Loewe |
|---|---|---|---|---|---|
| Cell Line | Status | Status | Activity | Sensitivity Score | Synergy Score |
| HSC2 | H1047R | GAIN | — | 80.3 | 10.4 |
| SCC9 | WT | GAIN, | HIGH | 60.7 | 10.1 |
| | | L133R | | | |
| CAL33 | H1047R | WT | — | 58.3 | 10 |
| SCC25 | GAIN | WT | — | 58.3 | 9.5 |
| FaDu | AMP | LOSS | — | 50.4 | 7.8 |
| BICR22 | AMP | WT | — | 74.8 | 7.35 |
| Detroit 562 | H1047R | WT | — | 51.5 | 7 |
| HSC3 | WT | GAIN | LOW | 6.9 | −2.1 |
| PECAPJ15 | WT | GAIN | LOW | 24.1 | −10.2 |
| SAS | WT | WT | LOW | 10.6 | −15.6 |

### Example 7: Cell Line Model without PIK3CA Alteration or Elevated HRAS Activity

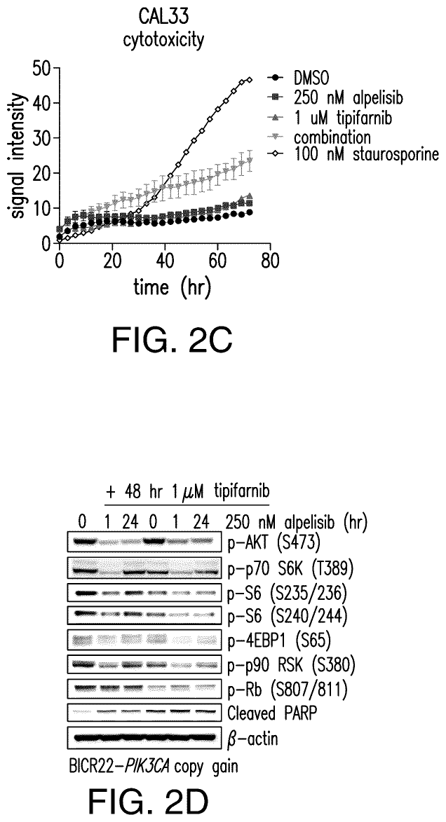

HSC3 cells (WT PIK3CA/HRAS) were treated with 250 nM alpelisib for 0, 1, or 24 hours in the presence or absence of 1 μM tipifarnib. Cells were treated with tipifarnib for 48 hours. Immunoblot of the indicated signaling proteins (representative of 3 biological replicates) are shown in FIG. 3A. HSC3 cells were treated with DMSO, 250 nM alpelisib, 1 μM tipifarnib, or the combination or the combination for 72 hours with 100 nM staurosporine as a positive control. Apoptosis (Annexin V; FIG. 3B) and cytotoxicity (DNA stain/loss of membrane integrity; FIG. 3C) over time were measured via Incucyte live cell imaging. Data represent means+/−SD of three biological replicates. Neither PARP cleavage (FIG. 3B) nor Annexin V/cytotoxicity (FIG. 3C) were significantly induced by the combination.

### Example 8: Tipifarnib Blocks Rheb Protein Localization to Lysosome

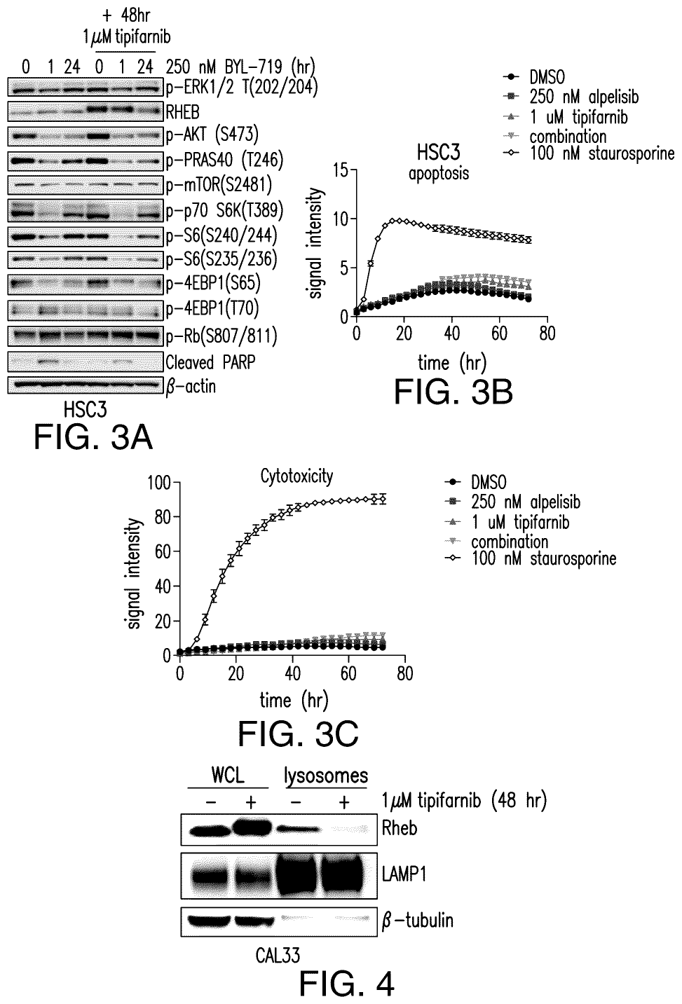

Active Rheb protein is thought to localize to lysosomes to regulate mTOR activity. Density gradient ultracentrifugation was utilized to extract lysosomes from CAL33 cells treated with DMSO or tipifarnib. Lysosomes were lysed and subjected to immunoblot analysis alongside whole-cell lysate (WCL), FIG. 4. LAMP1 is a lysosome-specific marker and is used in this assay. Tipifarnib treatment markedly diminished the lysosomal localization of Rheb protein as shown in FIG. 4.

### Example 9: IHC Assay for HRAS Overexpression in Head and Neck Cancers

Background. Evaluation of a commercial database of de-identified clinical and molecular data derived from >1000 HNSCC tumors and other comparative tumor types (Tempus, Chicago, IL) was performed to determine the prevalence of HRAS mutations and overexpression, the prevalence of PIK3CA alterations (mutations, amplification, overexpression), and the overlap between such biomarkers, in HNSCC.

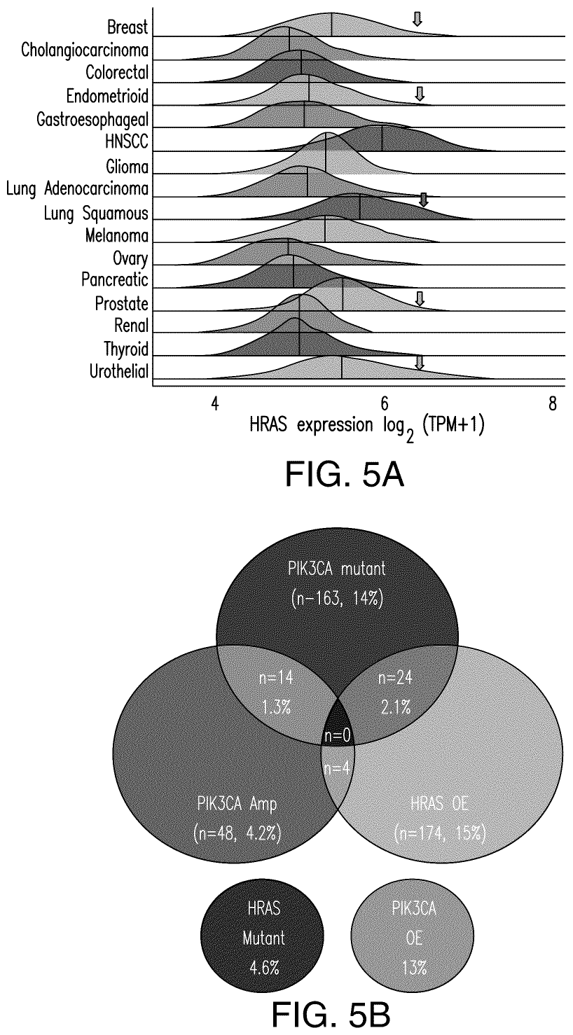

The distribution of HRAS expression by primary tissue site from the analysis of the database is shown in FIG. 5A. HRAS expression, as shown in FIG. 5A, was highest in HNSCC (n=1,119), while other tumors such as breast, endometrioid, lung squamous, prostate, and urothelial also contain a subset with high HRAS expression (arrows), possibly associated with squamous histology. Cervical squamous tumors have also been shown to exhibit HRAS overexpression. The genetic alterations or over-expression of the HRAS gene and the PIK3CA gene in HNSCC were also analyzed from the database, and are depicted in a Venn diagram as shown in FIG. 5B. In analyzing the database, HRAS overexpression was defined as an HRAS expression equal to or greater than 1 standard deviation above the mean (Z score ≥1). In an analysis of rates of mutation and amplification of PIK3CA and HRAS in HNSCCs from the Cancer Genome Atlas (TCGA) dataset, approximately 30% of head and neck tumors harbored mutations or amplification of PIK3CA, HRAS mutations occurred in approximately 6% of HNSCCs, and up to 15% of head and neck tumors exhibited high HRAS expression.

Overexpression of HRAS in HNSCC patients indicates that HRAS may be a viable target for biomarker-driven clinical studies for patients with recurrent/metastatic HNSCC. As such, a Clinical Laboratory Improvement Amendments (CLIA)-validated immunohistochemistry (IHC) assay was developed to assess HRAS expression in tumor biopsies.

IHC Assay Development and Procedure. To develop an IHC assay for clinical use, an antibody specific for HRAS isoform 1 (the mRNA of which includes the amino acid codons for the farnesylation motif of the protein, which allows for trafficking to the plasma membrane) was identified and validated. In brief, HEK293T cells over-expressing either N-Ras, H-Ras isoform 1, H-Ras isoform 2, K-Ras isoform a, or K-Ras isoform b were subjected to IHC staining with one of three antibodies to human HRAS: 1) mouse monoclonal (clone OTI1D9); 2) rabbit monoclonal (clone ARC0098); and 3) a rabbit polycolonal antibody. IHC staining was compared to staining in un-transfected HEK293T cells which served as the negative control. Antibody reactivity (i.e., staining) was graded as: “−” for no specific staining; “+” for minor staining; “++” for intermediate staining; or “+++” for strong staining. Results are provided in Table 5.

TABLE 5

| | | HEK293 | HEK293 | | HEK293 | HEK293 |
|---|---|---|---|---|---|---|
| | HEK293 | with HRAS | with HRAS | HEK293 | with KRAS | with KRAS |
| | (parental) | Isoform 1 | Isoform 2 | with NRAS | Isoform a | Isoform B |
| OTI1D9 | + | +++ | + | +++ | ++ | ++ |
| ARC0098 | − | +++ | − | − | − | − |
| Polyclonal | + | +++ | ++ | ++ | ++ | ++ |

Both the OTI1D9 clone from Origene and the rabbit polyclonal from ProteinTech were unable to differentiate between the various forms of RAS. Monoclonal antibody clone ARC0098 (“ARC0098”) only detected the HRAS isoform 1, but not isoform 2. ARC0098 did not detect NRAS, KRAS isoform a, or KRAS isoform b. Very low levels of staining with ARC0098 observed in the cell samples was also present in un-transfected HEK293T cells. Thus, the observed low-level ARC0098 staining was negative, background staining.

Validation of an IHC assay using the ARC0098 antibody clone was then performed. The IHC staining procedure used was as follows:

STEP 1) Slide Preparation: Formalin-fixed, paraffin-embedded (FFPE) human cancer tissues blocks were cut at 4-5 μm thickness and sections were mounted onto positively-charged glass slides. Slides were baked (60° C., dry heat) for 1 hour prior to Step 2. The appropriate Autostainer Link 48 program was loaded by selecting the appropriate protocol.

STEP 2) Dewaxing/Automated Antigen Retrieval. Tissue sections were de-waxed and pre-treated using the PT Link. Antigen/epitope retrieval (antigen unmasking) was performed using a Heat Induced Epitope Recovery (HIER) solution. Slides were loaded in racks and placed in pre-heated (65° C.) 1× High pH Target Retrieval Solution in the PT Link. The PT Link was heated to 97° C. for 20 minutes and automatically cooled to 65° C. After cooling, racks were placed in a container with 1×FLEX wash buffer for 1-5 minutes.

STEP 3) Automated Immunohistochemistry: All procedures were automated at room temperature using the Dako/Agilent Autostainer Link 48 platform. Slides were treated with Proteinase K (diluted in FLEX wash buffer), to further expose the epitopes for binding, on the Autostainer Link 48 platform. The Dako/Agilent Autostainer Link 48 platform protocol was ran as follows with intervening rinses of 1×FLEX wash buffer between each step: a) 10 minutes in Proteinase K, 300 μL per slide; b) 5 minutes in FLEX HP Block, 300 μL per slide; c) 60 minutes HRAS primary antibody diluted in Dako Antibody Diluent, 300 μL per slide; d) 20 minutes in FLEX HRP, 300 μL per slide; e) 5 minutes in FLEX wash buffer, 300 μL per slide; f) 10 minutes in FLEX DAB+ Substrate-Chromogen, 300 μL per slide; g) 5 minutes in FLEX Hematoxylin, 300 μL per slide; h) Rinse in Deionized (DI) Water; i) 5 minutes in FLEX wash buffer, 300 μL per slide.

STEP 4) Dehydration/Coverslipping. Slides were immersed in room temperature deionized water and transferred to the coverslip area. Slides were rinsed in distilled water and dehydrated with washes in an alcohol series (95%, 100% ethanol) and organic solvent (xylene, 100%, four changes). After dehydration, slides were coverslipped using non-aqueous semi-permanent mounting media.

A species-matched positive control (standard antibody) with established signal strength in control tissues was used in each IHC run (run control) to confirm proper detection reagent performance. The IHC run control used was CD3 (derived in rabbit) tested on formalin-fixed, paraffin-embedded (FFPE) control tonsil tissues. Rabbit IgG isotype-matched negative controls for the corresponding HRAS biomarker assay conditions were used to determine any non-specific staining inherent in the detection reagents or tissues and to define any potential background reactivity from these sources. Previously tested cancer tissues served as a control for HRAS reactivity during each IHC run. A multi-tissue block included different samples of head and neck (H&N) cancer and normal tonsil with various levels of HRAS expression as determined during previous testing.

Antibody Specifications: Monoclonal antibody ARC0098, a Rabbit IgG, Vendor Invitrogen (Catalog No. MA5-35323), synthesized peptide derived from human GTPase HRAS.

Next, to assess if differences exist between different commercial preparations of this antibody for IHC experimentation, lot variability testing was performed. In brief, lot variability testing was conducted using 2 different vendor lots of HRAS (ARC0098) antibody following the IHC procedures and scoring procedures as described above. The antibody lots were tested on FFPE serial sections of 8 different evaluable H&N cancer samples that represented various biomarker expression levels.

All HRAS scoring results for plasma membrane and cytoplasmic Percent Scores ≥3+ were identical between antibody lots. When taken together, the tests for the 8 evaluable H&N cancer samples with 2 antibody lots resulted in 16 concordant and 0 discordant results. As such, concordant staining was observed for different HRAS (ARC0098) lots by demonstrating a 95% CI of 100.0%±0.0%. That is, different preparations of HRAS (ARC008) antibody were highly congruent and acceptable for IHC testing.

The assay protocol was validated for sensitivity, specificity, and pathology concordance as described below. Throughout testing, samples that previously showed a range of HRAS (ARC0098) tumor staining served as positive/quality controls (QC) to demonstrate appropriate reactivity. Standard species-matched positive controls (Rabbit CD3) and negative controls (Rabbit IgG) were included throughout testing and reacted as expected. Samples were also stained with hematoxylin and eosin (H&E) for morphological assessment to assist in scoring by the pathologists.

Tumor Sensitivity Screening

Sensitivity testing for the HRAS (ARC0098) IHC was performed in a CLIA certified clinical laboratory on a panel of FFPE tumor tissues. The tumor screen was performed to understand the range of staining intensities and abundance (penetrance) of reactivity across a representative sample set from the head and neck (H&N) cancer indication. A total of 50 H&N cancer samples were tested: 46 samples contained histologic evidence of head and neck squamous cell carcinoma and 4 samples were normal/benign tissue from the oral cavity and larynx. Each evaluable cancer sample was scored for HRAS tumor cell staining by a board-certified pathologist according to the scoring scheme described above. Benign glands showed 1+ cytoplasmic staining, benign connective tissue showed 0-2+ cytoplasmic staining, and benign squamous epithelium ranged from 1-3+ plasma membrane staining. In samples with head and neck squamous cell carcinoma, areas of normal and tumor cells are easily distinguished on the H&E slide by the scoring pathologist.

Scoring results in H&N cancer samples, including Percent Scores ≥1+, ≥2+, ≥3+ and H-Scores, are shown in Table 6. Positive or negative status using the cut-off of plasma membrane or cytoplasmic Percent Score ≥3+ of ≥50 as positive (POS) is also listed in Table 6 (none of the H&N cancer samples evaluated showed a cytoplasmic tumor staining with a Percent Score ≥3+ above 25, and thus only the plasma membrane tumor staining results are shown in Table 6).

TABLE 6
HRAS Scoring in H&N Cancer

| | | HRAS Plasma Membrane Tumor Staining | | | | | | | | |
|---|---|---|---|---|---|---|---|---|---|---|
| Sample | | | | | | % | % | % | | |
| Info. | | % at Differential Intensities | | | | Staining | Staining | Staining | H- | POS/ |
| ID | Path. | 0 | 1+ | 2+ | 3+ | ≥1+ | 2+ | ≥3+ | Score | NEG |
| 1 | SQC | 40 | 20 | 20 | 20 | 60 | 40 | 20 | 120 | NEG |
| 2 | SQC | 20 | 30 | 30 | 20 | 80 | 50 | 20 | 150 | NEG |
| 3 | SQC | 0 | 60 | 40 | 0 | 100 | 40 | 0 | 140 | NEG |
| 4 | SQC | 0 | 0 | 60 | 40 | 100 | 100 | 40 | 240 | NEG |
| 5 | SQC | 10 | 60 | 30 | 0 | 90 | 30 | 0 | 120 | NEG |
| 6 | SQC | 5 | 10 | 25 | 60 | 95 | 85 | 60 | 240 | POS |
| 7 | SQC | 5 | 5 | 10 | 80 | 95 | 90 | 80 | 265 | POS |
| 8 | SQC | 0 | 0 | 40 | 60 | 100 | 100 | 60 | 260 | POS |
| 9 | HN | 0 | 0 | 0 | 100 | 100 | 100 | 100 | 300 | POS |
| 10 | HN | 25 | 25 | 25 | 25 | 75 | 50 | 25 | 150 | NEG |
| 11 | SQC | 0 | 20 | 40 | 40 | 100 | 80 | 40 | 220 | NEG |
| 12 | SQC | 0 | 0 | 0 | 100 | 100 | 100 | 100 | 300 | POS |
| 13 | SQC | 100 | 0 | 0 | 0 | 0 | 0 | 0 | 0 | NEG |
| 14 | SQC | 85 | 5 | 5 | 5 | 15 | 10 | 5 | 30 | NEG |
| 15 | SQC | 10 | 10 | 30 | 50 | 90 | 80 | 50 | 220 | POS |
| 16 | SQC | 0 | 10 | 30 | 60 | 100 | 90 | 60 | 250 | POS |
| 17 | SQC | 0 | 60 | 40 | 0 | 100 | 40 | 0 | 140 | NEG |
| 18 | SQC | 20 | 30 | 30 | 20 | 80 | 50 | 20 | 150 | NEG |
| 19 | SQC | 0 | 20 | 20 | 60 | 100 | 80 | 60 | 240 | POS |
| 20 | SQC | 10 | 30 | 30 | 30 | 90 | 60 | 30 | 180 | NEG |
| 21 | SQC | 0 | 40 | 30 | 30 | 100 | 60 | 30 | 190 | NEG |
| 22 | SQC | 0 | 20 | 60 | 20 | 100 | 80 | 20 | 200 | NEG |
| 23 | SQC | 0 | 0 | 0 | 100 | 100 | 100 | 100 | 300 | POS |
| 24 | SQC | 90 | 10 | 0 | 0 | 10 | 0 | 0 | 10 | NEG |
| 25 | SQC | 0 | 10 | 10 | 80 | 100 | 90 | 80 | 270 | POS |
| 26 | SQC | 100 | 0 | 0 | 0 | 0 | 0 | 0 | 0 | NEG |
| 27 | SQC | 0 | 0 | 0 | 100 | 100 | 100 | 100 | 300 | POS |
| 28 | SQC | 100 | 0 | 0 | 0 | 0 | 0 | 0 | 0 | NEG |
| 29 | SQC | 80 | 10 | 10 | 0 | 20 | 10 | 0 | 30 | NEG |
| 30 | SQC | 0 | 0 | 0 | 100 | 100 | 100 | 100 | 300 | POS |
| 31 | SQC | 0 | 30 | 40 | 30 | 100 | 70 | 30 | 200 | NEG |
| 32 | SQC | 70 | 10 | 10 | 10 | 30 | 20 | 10 | 60 | NEG |
| 33 | SQC | 0 | 0 | 0 | 100 | 100 | 100 | 100 | 300 | POS |
| 34 | SQC | 0 | 40 | 30 | 30 | 100 | 60 | 30 | 190 | NEG |
| 35 | SQC | 40 | 40 | 20 | 0 | 60 | 20 | 0 | 80 | NEG |
| 36 | SQC | 70 | 25 | 5 | 0 | 30 | 5 | 0 | 35 | NEG |
| 37 | SQC | 100 | 0 | 0 | 0 | 0 | 0 | 0 | 0 | NEG |
| 38 | SQC | 100 | 0 | 0 | 0 | 0 | 0 | 0 | 0 | NEG |
| 39 | SQC | 60 | 40 | 0 | 0 | 40 | 0 | 0 | 40 | NEG |
| 40 | SQC | 90 | 10 | 0 | 0 | 10 | 0 | 0 | 10 | NEG |
| 41 | SQC | 20 | 10 | 10 | 60 | 80 | 70 | 60 | 210 | POS |
| 42 | SQC | 60 | 20 | 10 | 10 | 40 | 20 | 10 | 70 | NEG |
| 43 | SQC | 0 | 0 | 0 | 100 | 100 | 100 | 100 | 300 | POS |
| 44 | SQC | 0 | 10 | 30 | 60 | 100 | 90 | 60 | 250 | POS |
| 45 | SQC | 60 | 10 | 10 | 20 | 40 | 30 | 20 | 90 | NEG |
| 46 | SQC | 0 | 30 | 40 | 30 | 100 | 70 | 30 | 200 | NEG |

Key: Path. = Pathologic Diagnosis;
% ≥ = % Staining ≥ listed grade;
POS = positive (Membrane or Cytoplasm Percent Score ≥3+ ≥50%);
NEG = negative (Membrane or Cytoplasm Percent Score ≥3+ <50%)
Note:
Four samples with no evaluable tumor (NET) not shown in table

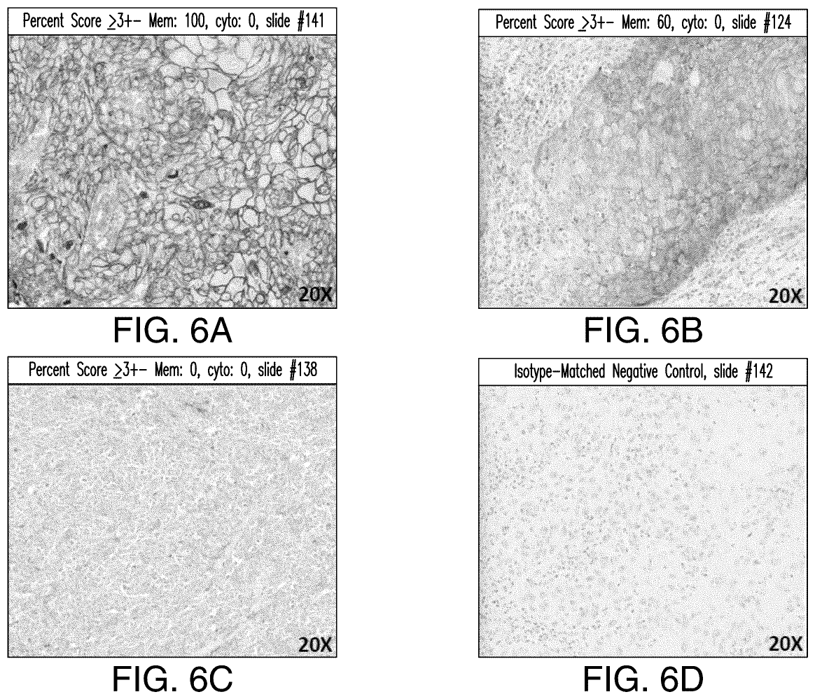

Representative images showing different levels of HRAS tumor expression in H&N cancer are shown in FIGS. 6A-6D. The images show only select areas of tissue according to the HRAS immunohistochemistry (IHC) assay disclosed herein using HRAS (ARC0098) & Rabbit IgG Staining in H&N Cancer: Percent Score ≥3+ with Plasma Membrane 100 and Cytoplasmic 0 (as shown in FIG. 6A); with Plasma Membrane 60, Cytoplasmic 0 (as shown in FIG. 6B); with Plasma Membrane 0, Cytoplasmic 0 (as shown in FIG. 6C), and an isotype-matched Negative Control is shown in FIG. 6D. However, the entire tumor region for each sample was evaluated to arrive at the Percent Scores ≥3+. Rabbit IgG isotype-matched negative controls were run in place of the HRAS (ARC0098) monoclonal antibody on each sample tested in the sensitivity screen. Results using these negative controls were nonreactive in each sample (data not shown).

As part of the standard analysis of sensitivity data and to aid in comparative evaluation of HRAS (ARC0098) tumor cell reactivity in the H&N cancer indication, summary tables based on Percent Scores [sum of percentages at different intensities (≥1+, ≥2+, and 3+)] and H-Scores [sum of each percentage score (0-100%) multiplied by its corresponding intensity score (0, 1+, 2+, 3+)] were prepared that divided the data for plasma membrane and cytoplasmic tumor cell staining according to different theoretical positivity thresholds. Thresholds for HRAS positivity based on plasma membrane tumor staining only on 46 H&N Cancer evaluable cases are presented in Table 7. Thresholds for HRAS positivity based on cytoplasmic tumor staining only on 46 H&N Cancer evaluable cases are presented in Table 8.

TABLE 7
HRAS (ARC0098) Thresholds for Plasma Membrane Tumor Reactivity Data (Staining) in H&N Cancer Screen

| ≥1+ Staining in | | ≥1+ Staining in | | ≥1+ Staining in | | ≥1+ Staining in | | ≥1+ Staining in | | ≥1+ Staining in | | |
|---|---|---|---|---|---|---|---|---|---|---|---|---|
| ≥1% Tumor Cells | | ≥10% Tumor Cells | | ≥20% Tumor Cells | | ≥50% Tumor Cells | | ≥75% Tumor Cells | | ≥90% Tumor Cells | | Average |
| No. of | % of | No. of | % of | No. of | % of | No. of | % of | No. of | % of | No. of | % of | Percent |
| Cases | Indication | Cases | Indication | Cases | Indication | Cases | Indication | Cases | Indication | Cases | Indication | Score ≥1+ |
| 41 | 89% | 41 | 89% | 38 | 83% | 32 | 70% | 30 | 65% | 26 | 57% | 70.2 |
| ≥2+ Staining in | | ≥2+ Staining in | | ≥2+ Staining in | | ≥2+ Staining in | | ≥2+ Staining in | | ≥2+ Staining in | | |
| ≥1% Tumor Cells | | ≥10% Tumor Cells | | ≥20% Tumor Cells | | ≥50% Tumor Cells | | ≥75% Tumor Cells | | ≥90% Tumor Cells | | Average |
| No. of | % of | No. of | % of | No. of | % of | No. of | % of | No. of | % of | No. of | % of | Percent |
| Cases | Indication | Cases | Indication | Cases | Indication | Cases | Indication | Cases | Indication | Cases | Indication | Score ≥2+ |
| 38 | 83% | 37 | 80% | 35 | 76% | 27 | 59% | 18 | 39% | 13 | 28% | 53.7 |
| ≥3+ Staining in | | ≥3+ Staining in | | ≥3+ Staining in | | ≥3+ Staining in | | ≥3+ Staining in | | ≥3+ Staining in | | |
| ≥1% Tumor Cells | | ≥10% Tumor Cells | | ≥20% Tumor Cells | | ≥50% Tumor Cells | | ≥75% Tumor Cells | | ≥90% Tumor Cells | | Average |
| No. of | % of | No. of | % of | No. of | % of | No. of | % of | No. of | % of | No. of | % of | Percent |
| Cases | Indication | Cases | Indication | Cases | Indication | Cases | Indication | Cases | Indication | Cases | Indication | Score ≥3+ |
| 32 | 70% | 31 | 67% | 29 | 63% | 16 | 35% | 9 | 20% | 7 | 15% | 35.9 |

| H-Score ≥1 | | H-Score ≥10 | | H-Score ≥50 | | H-Score ≥100 | | H-Score ≥200 | | H-Score ≥250 | | |
|---|---|---|---|---|---|---|---|---|---|---|---|---|
| No. of | % of | No. of | % of | No. of | % of | No. of | % of | No. of | % of | No. of | % of | Average |
| Cases | Indication | Cases | Indication | Cases | Indication | Cases | Indication | Cases | Indication | Cases | Indication | H-Score |
| 41 | 89% | 41 | 89% | 35 | 76% | 31 | 67% | 21 | 46% | 12 | 26% | 159.8 |

TABLE 8
HRAS (ARC0098) Thresholds for Cytoplasmic Tumor Reactivity Data (Staining) in H&N Cancer Screen

| ≥1+ Staining in | | ≥1+ Staining in | | ≥1+ Staining in | | ≥1+ Staining in | | ≥1+ Staining in | | ≥1+ Staining in | | |
|---|---|---|---|---|---|---|---|---|---|---|---|---|
| ≥1% Tumor Cells | | ≥10% Tumor Cells | | ≥20% Tumor Cells | | ≥50% Tumor Cells | | ≥75% Tumor Cells | | ≥90% Tumor Cells | | Average |
| No. of | % of | No. of | % of | No. of | % of | No. of | % of | No. of | % of | No. of | % of | Percent |
| Cases | Indication | Cases | Indication | Cases | Indication | Cases | Indication | Cases | Indication | Cases | Indication | Score ≥1+ |
| 45 | 98% | 45 | 98% | 43 | 93% | 29 | 63% | 18 | 39% | 14 | 30% | 62.0 |
| ≥2+ Staining in | | ≥2+ Staining in | | ≥2+ Staining in | | ≥2+ Staining in | | ≥2+ Staining in | | ≥2+ Staining in | | |
| ≥1% Tumor Cells | | ≥10% Tumor Cells | | ≥20% Tumor Cells | | ≥50% Tumor Cells | | ≥75% Tumor Cells | | ≥90% Tumor Cells | | Average |
| No. of | % of | No. of | % of | No. of | % of | No. of | % of | No. of | % of | No. of | % of | Percent |
| Cases | Indication | Cases | Indication | Cases | Indication | Cases | Indication | Cases | Indication | Cases | Indication | Score ≥2+ |
| 22 | 48% | 22 | 48% | 16 | 35% | 6 | 13% | 1 | 2% | 1 | 2% | 15.8 |
| ≥3+ Staining in | | ≥3+ Staining in | | ≥3+ Staining in | | ≥3+ Staining in | | ≥3+ Staining in | | ≥3+ Staining in | | |
| ≥1% Tumor Cells | | ≥10% Tumor Cells | | ≥20% Tumor Cells | | ≥50% Tumor Cells | | ≥75% Tumor Cells | | ≥90% Tumor Cells | | Average |
| No. of | % of | No. of | % of | No. of | % of | No. of | % of | No. of | % of | No. of | % of | Percent |
| Cases | Indication | Cases | Indication | Cases | Indication | Cases | Indication | Cases | Indication | Cases | Indication | Score ≥3+ |
| 7 | 15% | 6 | 13% | 5 | 11% | 0 | 0% | 0 | 0% | 0 | 0% | 2.6 |

| H-Score ≥1 | | H-Score ≥10 | | H-Score ≥50 | | H-Score ≥100 | | H-Score ≥200 | | H-Score ≥250 | | |
|---|---|---|---|---|---|---|---|---|---|---|---|---|
| No. of | % of | No. of | % of | No. of | % of | No. of | % of | No. of | % of | No. of | % of | Average |
| Cases | Indication | Cases | Indication | Cases | Indication | Cases | Indication | Cases | Indication | Cases | Indication | H-Score |
| 45 | 98% | 45 | 98% | 33 | 72% | 19 | 41% | 1 | 2% | 0 | 0% | 80.3 |

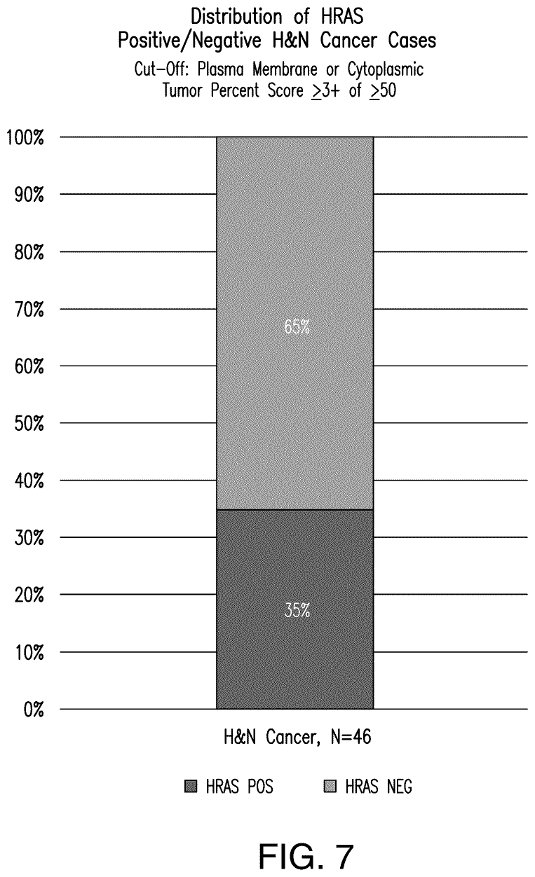

A cut-off for HRAS (ARC0098) positivity was selected that considers plasma membrane or cytoplasmic tumor staining as follows: Plasma Membrane Percent Score ≥3+(consisting of 3+ intensities) of ≥50 or Cytoplasmic Percent Score ≥3+(consisting of 3+ intensities) of ≥50=Positive. Using this cut-off to assess the 46 evaluable H&N cancer samples in the tumor screen, 35% (16/46) were positive (i.e., with 50% or more cells having a Percent Score ≥3+ Plasma Membrane or Cytoplasmic) and 65% (30/46) were negative. This breakdown is depicted in FIG. 7. In the tumor screen, no samples showed cytoplasmic tumor staining with Percent Score ≥3+ above 25. As such, only plasma membrane tumor staining contributed to the positivity determinations for this data set.

Specificity Testing in Normal Tissues

The specificity of the HRAS (ARC0098) IHC assay to its target was determined using a normal human tissue microarray (TMA) that included 96 different FDA-recommended tissues. The TMA included multi-normal human tissues (96 samples, 35 organs/sites from 3 individuals, 1.5 mm). Sections of all normal tissue samples were histology-stained with hematoxylin and eosin (H&E) and IHC-stained with HRAS (ARC0098) and rabbit IgG negative control. All stained normal tissues were assessed by board-certified pathologist for HRAS (ARC0098) staining in normal tissue components according to the methods and scoring scheme described above. Generally, HRAS staining in the normal tissue components was below the threshold used in the IHC assay for positive detection of HNSCC.

Pathology Concordance Testing

HRAS (ARC0098) staining was observed in a subset of tumor cells where it localized to the plasma membrane and the cytoplasm. Pathology analyses and HRAS (ARC0098) plasma membrane and cytoplasmic tumor staining were evaluated by board-certified pathologists using H-Scores and/or Percent Scores according to the scoring methods described as follows:
For tumor cell evaluation, HRAS staining is scored separately for plasma membrane and cytoplasmic staining. Staining in tumor cells is evaluated semi-quantitatively for each localization.
The pathologist only provides semi-quantitative scores for HRAS expression in tumor cells. That is, numeric scoring excludes any signal in surrounding stroma and areas of non-tumor.
Areas of ischemic changes, thermal artifact, and necrosis are also not scored.
Each tissue is stained with H&E to assist the pathologist in sample evaluation during scoring.
The main components for scoring HRAS expression in tumor cells include percentages at differential intensities to determine Percent Scores and H-Scores (described below).
The percentage of tumor cells with HRAS staining are recorded at a corresponding differential intensity on a four-point semi-quantitative scale (0, 1+, 2+, 3+). On this scale: 0=null, negative or non-specific staining, 1+=low or weak staining, 2+=medium or moderate staining, and 3+=high or strong staining.
The percentage of tumor cells staining at each intensity is estimated directly and typically reported as one of the following; though other increments can also be used at the pathologist's discretion: 0, 1, 2, 3, 5, 10, 15, 20, 25, 30, 35, 40, 45, 50, 55, 60, 65, 70, 75, 80, 85, 90, 95, 97, 98, 99, or 100%.

Analysis and scoring for HRAS expression included determination of Percent scores and H-scores. For the percent score method, tumor percent scores were calculated by summing the percentages of intensities at either ≥1+, ≥2+ or ≥3+. Thus, scores range from 0 to 100.
Percent Score ≥1+=(% at 1+)+(% at 2+)+(% at 3+)
Percent Score ≥2+=(% at 2+)+(% at 3+)
Percent Score ≥3+=(% at 3+)

For the H-Score method, the tumor H-score was calculated by summing the percentage of cells with intensity of expression (brown staining) multiplied by their corresponding differential intensity on a four-point semi-quantitative scale (0, 1+, 2+, 3+). Thus, scores ranged from 0 to 300.

H-Score=[(% at <1)×0]+[(% at 1+)×1]+[(% at 2+)×2]+[(% at 3+)×3]

A positivity cut-off for HRAS positivity was applied to confidence interval (CI) assessments to demonstrate positive/negative agreement for acceptable concordance in the validation studies herein. The positivity cut-off is also intended for use in clinical sample testing. The cut-off is described below:
Positivity Cut-Off: Plasma Membrane Percent Score ≥3+(consisting of 3+ intensities) of ≥50 OR Cytoplasmic Percent Score ≥3+(consisting of 3+ intensities) of ≥50=Positive.

Pathology concordance was tested to assess the consistency of HRAS (ARC0098) scoring between different pathologists (inter-reader scoring) and between different scoring sessions for the same pathologist (intra-reader scoring). Concordant scoring was observed among the pathologists and scoring sessions by demonstrating an appropriate 95% CI (confidence interval) when considering positive/negative agreement using the described positivity cut-offs for HRAS (ARC0098).

### Example 10: Tipifarnib and Alpelisib Activity in PIK3CA-Mutant Cell Line-Derived Xenograft Models

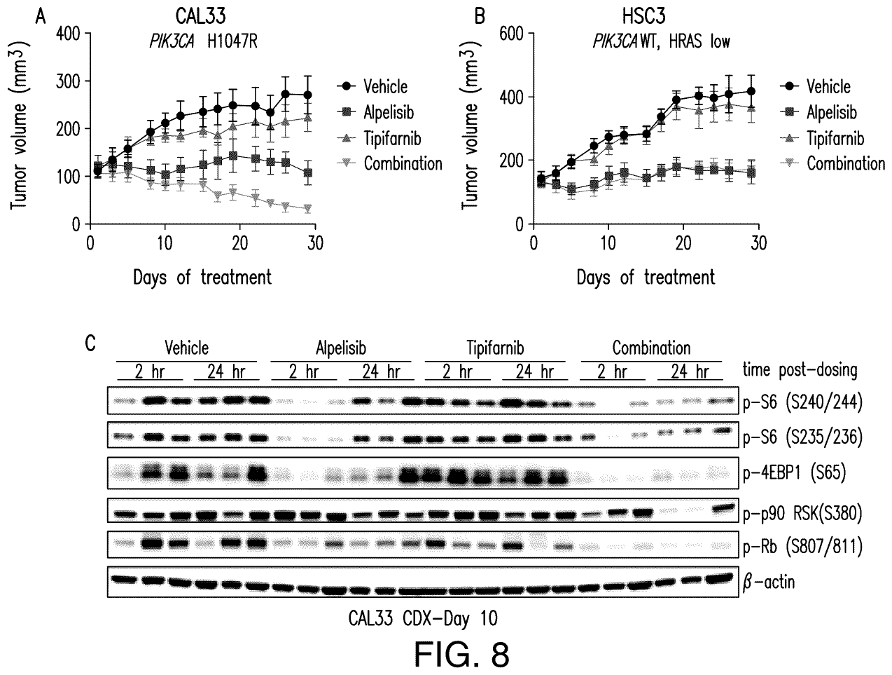

PIK3CA-mutant (CAL33) and PIK3CA WT/HRAS-low (HSC3) xenografts were implanted in athymic mice and were allowed to reach 200 mm³, at which time treatment vehicle, alpelisib (40 mg/kg QD), tipifarnib (60 mg/kg BID), or the combination (n=10 mice per group), was initiated and continued for 30 days. The combination blocked the growth of the PIK3CA-mutant tumors and induced tumor regression better than either agent alone (FIG. 8A). No additive effect was observed in the control PIK3CA WT/HRAS-low tumors (FIG. 8B).

To assess the underlying mechanism of tumor regression, the PIK3CA-mutant CAL33 tumors were treated with tipifarnib (60 mg/kg BID), alpelisib (40 mg/kg QD), or the combination for 10 days. On day 10, tumors were collected from all cohorts at 2 and 24 hours after the final dose of alpelisib for immunohistochemistry and immunoblot analysis. Immunoblot analysis of HRAS and RHEB in the CAL33 xenograft tumors showed that tipifarnib treatment resulted in defarnesylation of HRAS and RHEB (2 tumors per treatment group). Immunoblot analysis of signaling proteins (tumors from 3 animals per treatment group/collection timepoint) showed that alpelisib alone transiently inhibited mTOR activity (4EBP1, S6 phosphorylation at 2 hours), but by 24 hours, a significant rebound was observed (FIG. 8C). Addition of tipifarnib blocked this rebound and promoted cell cycle arrest as indicated by decreased Rb phosphorylation. Immunohistochemical analysis of cleaved PARP, caspase 3, and Ki67 in the collected CAL33 tumors revealed the tipifarnib-alpelisib doublet inhibited tumor proliferation (observed by Ki67 staining) and enhanced induction of apoptosis (PARP/caspase 3 cleavage) compared to single agent exposure. The combination of alpelisib and tipifarnib durably inhibited mTORC1 activity in vivo, leading to cell death and tumor regression.

### Example 11: Tipifarnib Effect on Signaling Proteins

The contribution of obligately farnesylated signaling proteins (HRAS, RHEB, p-AKT, p-p70 S6K (T389), p-S6 (S240/244), p-S6 (S235/236), p-4EBP1 (S65), p-MED (S217/221), p-ERK1/2 (T202/204), p-p90 RSK (S380)) to the sensitivity of tipifarnib and alpelisib was investigated. Upon farnesyltransferase inhibition, these proteins are defarnesylated and lose their membrane localization and activity.

CAL33, BICR22, and SCC9 cells were treated with DMSO, 250 nM alpelisib (24 hr), 1 μM tipifarnib (48 hr), or the combination. Using subcellular fractionation, immunoblot analysis of HRAS and RHEB in the cytosol (p-tubulin control) and membrane (LAMP1 control) fractions showed that tipifarnib treatment resulted in the membrane delocalization of the majority of HRAS and RHEB in all three cell types. This result suggests that these proteins were sufficiently defarnesylated to impair their activity.

In a separate study, CAL33 (PIK3CA H1047R), BICR22 (PIK3CA Copy Gain), and SCC9 (HRAS high) cells were transfected with small interfering RNAs to deplete RHEB and HRAS expression (vs. control non-targeting pool). After 48 hours to allow for knockdown, the cells were treated with 250 nM alpelisib. Cells were collected and lysed after 0, 1, or 24 h of alpelisib treatment and immunoblot analysis was performed. As with tipifarnib treatment, reduction of RHEB and HRAS expression decreased basal phosphorylation of S6K, S6, and 4EBP1 to varying degrees across the PIK3CA-mutant/amplified and high active cell lines. Inhibition of phospho-S6K, phospho-S6, and phospho-4EBP1 by alpelisib was greater in the double knockdown (siRHEB and siHRAS) cells compared to control-transfected cells, and this inhibition was more durable, exhibiting minimal rebound of phosphorylation after 24 hours. The effect of co-depletion of RHEB and HRAS expression on mTOR substrate phosphorylation was greatest in PIK3CA-mutant cells, whereas its effect on MAPK activity was largest in HRAS-high cells, pointing to a potential hierarchy of target dependence corresponding to genotype. The genetic targeting of HRAS and RHEB closely mimicked tipifarnib treatment in these models, indicating these are key farnesylation-dependent proteins in the context of PI3Kα inhibition in HNSCC.

### Example 12: Effect of Combination of Tipifarnib and Alpelisib in Patient-Derived Xenograft Models

PIK3CA-mutant, PIK3CA-amplified, or HRAS-overexpressing patient-derived xenograft (PDX) models (Crown Bioscience, Beijing, China) were prepared using the methods described in Example 4. Models were treated with vehicle, tipifarnib (60 mg/kg BID), alpelisib (40 mg/kg QD), or the combination on a continuous schedule. HRAS mRNA expression levels (log 2 FKPM) for xenograft models tested in this experiment are shown in Table 9 (see https://crownbio.com). Lines with HRAS mRNA expression (log 2 FKPM) above approx. 7.0 were characterized as HRAS high.

TABLE 9

| Cell Line | Type | Exp Level |
|---|---|---|
| HN3690 | PIK3CA^{E545K}; HRAS^{WT-high} | 7.22 |
| HN2593 | PIK3CA^{G118D}; HRAS^{WT-high} | 7.39 |
| HN0626 | PIK3CA^{H1047L-AMP}; HRAS^{WT} | 6.77 |
| HN3067 | PIK3CA^{AMP}; HRAS^{WT-high} | 7.04 |
| HN3540 | PIK3CA^{AMP}; HRAS^{WT} | 6.15 |
| HN2574 | PIK3CA^{AMP}; HRAS^{WT} | 6.13 |
| HN2594 | HRAS^{WT-high} | 7.11 |
| HN2576 | HRAS^{WT-high} | 7.22 |
| HN3411 | HRAS^{WT-high} | 7.64 |

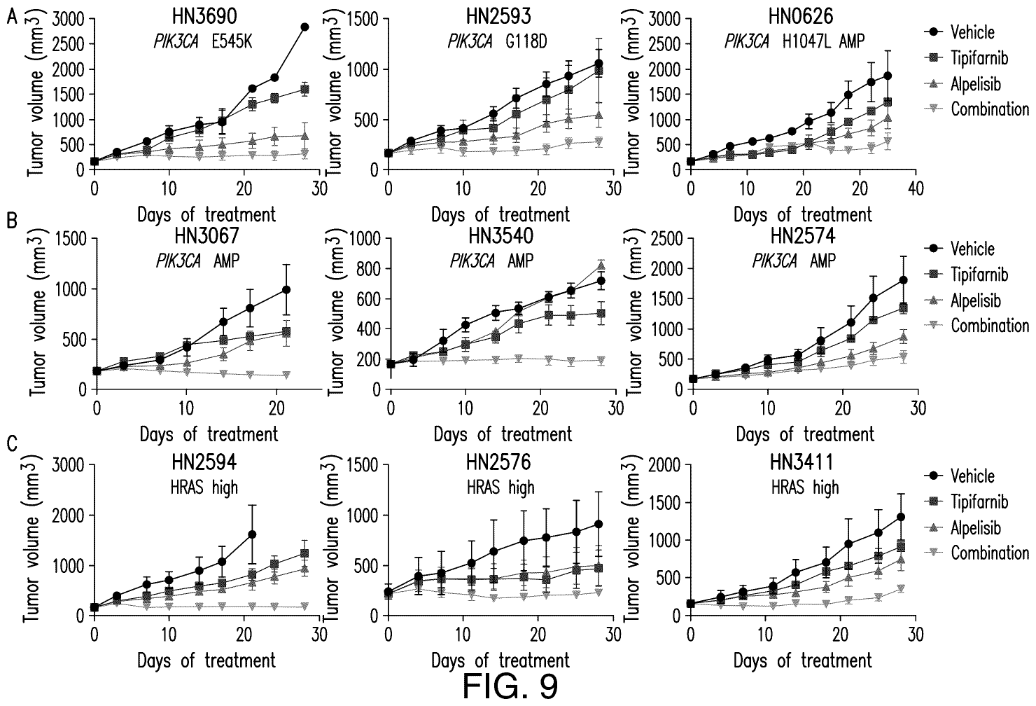

As shown in FIGS. 9A-C, combination treatment consistently slowed or blocked tumor growth more effectively than alpelisib or tipifarnib alone, and induced tumor regressions in several models. Data represent means+/−SEM, n=3 mice per group.

### Example 13: Mechanistic Studies

Using methods analogous to those described above, the following additional experiments were conducted.

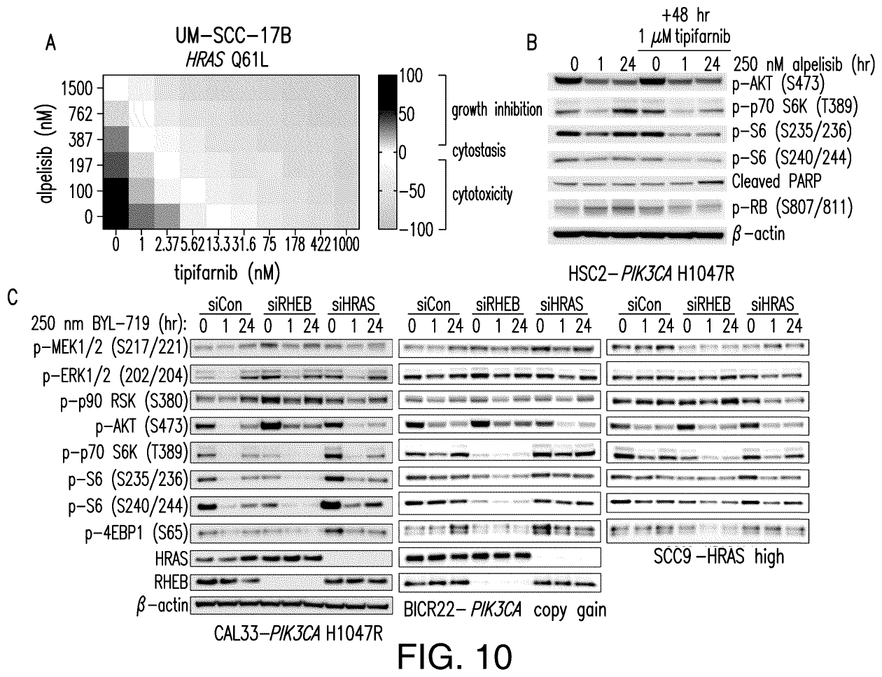

Inhibition of HRAS-mutant cell line spheroid growth by tipifarnib and/or alpelisib. The HRAS-mutant HNSCC cell line UM-SCC-17B (Q61L) was cultured as 3D tumor spheroids and treated with tipifarnib and/or alpelisib in a checkerboard dose-response fashion for 7 days. Both single agent tipifarnib and combined tipifarnib and alpelisib induced cytotoxicity in this cell line (FIG. 10A).

PI3K-mTOR Signaling in PIK3CA-mutant HSC2 Cell Line. The PIK3CA-mutant HNSCC cell line HSC2 (H1047R) was treated with the tipifarnib-alpelisib combination and mTOR activity was assessed by immunoblot. While mTOR activity, as evidenced by phospho-S6K and S6, was transiently inhibited by alpelisib alone at 1 h, the activity rebounded by 24 h (FIG. 10B). In contrast, minimal rebound of p-S6K and p-S6, and induction of cell death (PARP cleavage) was observed when cells were exposed to the tipifarnib/alpelisib combination.

Genetic Depletion of HRAS or RHEB Expression on MAPK and mTOR Signaling. To assess the individual contributions of RHEB and HRAS inhibition to the phenotype observed in HNSCC cells upon exposure to tipifarnib, RHEB and HRAS expression was depleted using siRNA in CAL33, BICR22, and SCC9 cells. Depletion of RHEB expression lowered basal levels of mTOR substrate phosphorylation, and blocked the rebound of their phosphorylation after 24 hours of alpelisib exposure (FIG. 10C).

### Example 14: Immunohistochemistry of CAL33 CDX Tumor Samples

CAL33 xenograft tumors treated with alpelisib (40 mg/kg QD), tipifarnib (60 mg/kg BID), or the combination, for 10 days were subjected to immunohistochemistry analysis to assess levels of cell proliferation and apoptosis.

Fixation of tumor samples was performed by zinc formalin (Z-Fix, Anatech) and tissues were paraffin-embedded; samples were sectioned into 5 μm sections. Samples were deparaffinized, hydrated with graded ethanols, and the endogenous peroxidase was blocked with 3% H₂O₂ in 70% ethanol. After washing with distilled water, antigen retrieval was performed with IHC antigen retrieval solution (ThermoFisher Scientific, #00-4955-58) in a microwave at the high setting. Slides were then washed with water and PBS, and incubated with the primary and secondary antibodies, and developed with the ABC reagent (Vector Laboratories, #PK-6100) and the DAB substrate kit (Vector Laboratories, #SK-4105). The following antibodies were used: Cleaved PARP (Cell Signaling Technology #9661, 1:400 dilution) and Ki67 (DACO #M7240, 1:50). Percent positive cells in three fields per slide were counted using a microscope.

The tipifarnib-alpelisib combination inhibited tumor proliferation (Ki67 staining) and induced apoptosis. Results are shown in Table 10.

TABLE 10

| | Ki67 | Cleaved PARP |
|---|---|---|
| | (% positive cells) | (% positive cells) |
| Vehicle | 78.7 ± 0.9 | 0.6 ± 0.4 |
| Alpelisib | 53.7 ± 9.8 | 5.1 ± 0.4 |
| Tipifarnib | 35.1 ± 8.1 | 4.9 ± 2.1 |
| Combination | 1.7 ± 0.2 | 16.9 ± 6.7 |

### 5.1 Exemplary Embodiments

One or more than one (including for instance all) of the following exemplary Embodiments may comprise each of the other embodiments or parts thereof.

A1. A method of treating head and neck squamous cell carcinoma (HNSCC) in a patient, comprising administering to the patient (a) tipifarnib and (b) a PI3K inhibitor, preferably alpelisib, such as 10-400 mg of alpelisib.

A2. A method of mitigating, slowing the progression of, or overcoming cetuximab-resistance in a patient having HNSCC, comprising administering to the HNSCC patient (a) tipifarnib and (b) a PI3K inhibitor, preferably alpelisib; wherein the HNSCC patient is currently being treated, or was previously treated, with cetuximab.

A3. A method of preventing or delaying emergence of cetuximab-resistance in a patient having HNSCC, comprising administering to the HNSCC patient (a) tipifarnib and (b) a PI3K inhibitor, preferably alpelisib; wherein the patient is a cetuximab-naïve subject.

A4. The method of any one of Embodiments A1-A3, wherein the method improves the efficacy of tipifarnib.

A5. The method of any one of Embodiments A1-A4, wherein the method improves the efficacy of the PI3K inhibitor, preferably alpelisib.

A6. The method of any one of Embodiments A1-A5, wherein the method delays, halts or prevents progression of HNSCC.

A7. The method of any one of Embodiments A1-A6, wherein the method mitigates or reduces the severity of at least one symptom associated with HNSCC.

A8. The method of any one of Embodiments A1-A7, wherein the method mitigates, slows the progression of, or overcomes PI3K inhibitor-resistance or cetuximab-resistance.

A9. The method of any one of Embodiments A1-A8, wherein the method reduces or mitigates toxicities associated with the PI3K inhibitor or cetuximab.

A10. The method of any one of Embodiments A1-A9, wherein the HNSCC has an HRas protein mutation.

A11. The method of any one of Embodiments A1-A10, wherein the HNSCC is an HRAS-mutant dependent HNSCC.

A12. The method of Embodiment A11, wherein the HRAS mutation is or comprises a modification in a codon that encodes an amino acid substitution at a specific position selected from a group consisting of G12, G13, Q61, Q22, K117, A146, and any combination thereof, in the corresponding mutant HRas protein.

A13. The method of any one of Embodiments A1-A12, wherein the HNSCC has an overexpression of wild-type HRas protein.

A14. The method of any one of Embodiments A1-A13, wherein the HNSCC is a PIK3CA-dependent HNSCC.

A15. The method of any one of Embodiments A1-A14, wherein the HNSCC has a PIK3CA alteration or dysregulation.

A16. The method of Embodiment A15, wherein the PIK3CA alteration or dysregulation is a PIK3CA mutation.

A17. The method of Embodiment A16, wherein the PIK3CA mutation is or comprises a modification in a codon that encodes an amino acid substitution at a specific position selected from a group consisting of R38, E39, E78, R88, R93, E103, P104, V105, G106, R108, E109, E110, K111, G118, P124, E218, V344, N345, D350, G364, E365, P366, C378, C420, P447, P449, H450, G451, E453, P471, P539, E542, E545, Q546, D549, E579, E600, C604, S629, V638, C901, G914, D939, E970, M1004, G1007, Y1021, T1025, D1029, E1037, M1043, N1044, H1047, G1049, A1066, and N1068, and any combination thereof, in the corresponding mutant PI3K protein.

A18. The method of Embodiment A16 or Embodiment A17, wherein the PIK3CA mutation is or comprises a modification in a codon that encodes an amino acid substitution at a specific position selected from a group consisting of G118, C420, E542, E545, Q546, H1047, and any combination thereof, in the corresponding mutant PI3K protein.

A19. The method of any one of Embodiments A15-A18, wherein the PIK3CA alteration or dysregulation is a PIK3CA gene amplification.

A20. The method of any one of Embodiments A15-A19, wherein the PIK3CA alteration or dysregulation is a PIK3CA copy gain.

Embodiments A21-A30 intentionally left blank

A31. The method of any one of Embodiments A1-A30, wherein the HNSCC is early stage, advanced, relapsed, refractory, or metastatic HNSCC.

A32. The method of any one of Embodiments A1-A31, wherein the HNSCC is advanced HNSCC.

A33. The method of any one of Embodiments A1-A32, wherein the HNSCC is relapsed or refractory HNSCC.

A34. The method of any one of Embodiments A1-A33, wherein the HNSCC is metastatic HNSCC.

A35. The method of any one of Embodiments A1-A34, wherein the HNSCC is recurrent/metastatic HNSCC.

A36. The method of any one of Embodiments A1-A31, wherein the HNSCC is early stage HNSCC.

A37. The method of any one of Embodiments A1-A36, wherein the HNSCC is HPV-negative HNSCC.

A38. The method of any one of Embodiments A1-A37, wherein the HNSCC patient is currently being treated, or has been previously treated, with a first-line therapy for HNSCC.

A39. The method of Embodiment A38, wherein the method improves the efficacy of the first-line therapy.

A40. The method of any one of Embodiments A1-A39, wherein the HNSCC patient is currently being treated, or has been previously treated, with a second-line therapy for HNSCC.

A41. The method of Embodiment A40, wherein the method improves the efficacy of the second-line therapy.

A42. The method of any one of Embodiments A1-A41, wherein the HNSCC patient is currently being treated, or has been previously treated, with an anti-EGFR antibody therapy for HNSCC.

A43. The method of Embodiment A42, wherein the method improves the efficacy of the anti-EGFR antibody therapy.

A44. The method of any one of Embodiments A1-A43, wherein the HNSCC patient is currently being treated, or has been previously treated, with an immunotherapy for HNSCC.

A45. The method of Embodiment A44, wherein the method improves the efficacy of the immunotherapy.

A46. The method of any one of Embodiments A1-A45, wherein the HNSCC patient is currently being treated, or has been previously treated, with a localized or loco-regional disease therapies for HNSCC.

A47. The method of Embodiment A46, wherein the method improves the efficacy of the localized and loco-regional disease therapy.

A48. The method of any one of Embodiments A1-A45, wherein the HNSCC patient has received at least one prior treatment selected from the group consisting of first-line therapy, second-line therapy, anti-EGFR antibody therapy, immunotherapy, or localized and loco-regional disease therapy.

A49. The method of Embodiment A48, wherein the at least one prior treatment failed to treat the HNSCC.

A50. The method of Embodiment A48, wherein the at least one prior treatment failed to delay, halt or prevent progression of the HNSCC.

A51. The method of Embodiment A48, wherein the at least one prior treatment failed to mitigate or reduce the severity of at least one symptom associated with the HNSCC.

Embodiments A52-A55 intentionally left blank

A56. The method of any one of Embodiments A1-A55, wherein the tipifarnib and the PI3K inhibitor, preferably alpelisib are administered concurrently or sequentially.

A57. The method of any one of Embodiments A1-A56, wherein the tipifarnib is administered before the administration of the PI3K inhibitor, preferably alpelisib.

A58. The method of any one of Embodiments A1-A56, wherein the tipifarnib is administered after the administration of the PI3K inhibitor, preferably alpelisib.

A59. The method of any one of Embodiments A1-A58, wherein the tipifarnib is administered to the patient orally.

A60. The method of any one of Embodiments A1-A59, wherein the tipifarnib is administered at a dose of 0.01-50 mg/kg body weight per day.

A61. The method of any one of Embodiments A1-A60, wherein the tipifarnib is administered at a dose of 1-2400 mg per day.

A62. The method of any one of Embodiments A1-A61, wherein the tipifarnib is administered at a dose of about 100 mg, about 200 mg, about 300 mg, about 400 mg, about 600 mg, about 900 mg, about 1200 mg per day, or about 1800 mg per day.

A63. The method of any one of Embodiments A1-A62, wherein the tipifarnib is administered at a dose of about 300 mg per day.

A64. The method of any one of Embodiments A1-A62, wherein the tipifarnib is administered at a dose of about 600 mg per day.

A65. The method of any one of Embodiments A1-A62, wherein the tipifarnib is administered at a dose of about 900 mg per day.

A65A. The method of any one of Embodiments A1-A62, wherein the tipifarnib is administered at a dose of about 1200 mg per day.

A65B. The method of any one of Embodiments A1-A62, wherein the tipifarnib is administered at a dose of about 1800 mg per day.

A66. The method of any one of Embodiments A1-A65B, wherein the dose of the tipifarnib is administered 1, 2, 3, or 4 times per day.

A67. The method of any one of Embodiments A1-A66, wherein the dose of the tipifarnib is administered twice per day.

A68. The method of any one of Embodiments A1-A66, wherein the dose of the tipifarnib is administered once per day.

A69. The method of any one of Embodiments A1-A68, wherein the PI3K inhibitor, preferably alpelisib, is administered to the patient orally.

A70. The method of any one of Embodiments A1-A69, wherein the PI3K inhibitor, preferably alpelisib, is administered to the patient at a dose of about 50 mg, about 100 mg, about 150 mg, about 200 mg, about 250 mg, or about 300 mg.

A71. The method of any one of Embodiments A1-A69, wherein the PI3K inhibitor, preferably alpelisib, is administered to the patient at a dose of about 50 mg.

A72. The method of any one of Embodiments A1-A69, wherein the PI3K inhibitor, preferably alpelisib, is administered to the patient at a dose of about 150 mg.

A73. The method of any one of Embodiments A1-A69, wherein the PI3K inhibitor, preferably alpelisib, is administered to the patient at a dose of about 150 mg.

A74. The method of any one of Embodiments A1-A69, wherein the PI3K inhibitor, preferably alpelisib, is administered to the patient at a dose of about 200 mg.

A75. The method of any one of Embodiments A1-A69, wherein the PI3K inhibitor, preferably alpelisib, is administered to the patient at a dose of about 250 mg.

A76. The method of any one of Embodiments A1-A69, wherein the PI3K inhibitor, preferably alpelisib, is administered to the patient at a dose of about 300 mg.

A77. The method of any one of Embodiments A1-A69, wherein the PI3K inhibitor, preferably alpelisib, is administered to the patient at a dose of about 400 mg.

A78. The method of any one of Embodiments A1-A77, wherein the dose of the PI3K inhibitor, preferably alpelisib, is administered to the patient once or twice per day.

A79. The method of any one of Embodiments A1-A77, wherein the dose of the PI3K inhibitor, preferably alpelisib, is administered once per day.

A80. The method of any one of Embodiments A1-A79, wherein the tipifarnib is administered once or twice per day, preferably twice per day, on days 1-7 of a 28-day cycle and the PI3K inhibitor, preferably alpelisib, is administered once per day of a 28-day cycle.

A81. The method of any one of Embodiments A1-A79, wherein the tipifarnib is administered once or twice per day, preferably twice per day, on days 1-7 and 15-21 of a 28-day cycle and the PI3K inhibitor, preferably alpelisib, is administered once per day of a 28-day cycle.

A82. The method of any one of Embodiments A1-A79, wherein the tipifarnib is administered once or twice per day, preferably twice per day, on days 1-21 of a 28-day cycle and the PI3K inhibitor, preferably alpelisib, is administered once per day of a 28-day cycle.

A83. The method of any one of Embodiments A1-A79, wherein the tipifarnib and the PI3K inhibitor, preferably alpelisib, are administered once or twice per day of a 28-day cycle.

A84. The method of any one of Embodiments A1-A83, wherein the tipifarnib and the PI3K inhibitor, preferably alpelisib, are administered for one or more 28-day cycles.

A85. The method of any one of Embodiments A1-A84, wherein the tipifarnib and the PI3K inhibitor, preferably alpelisib, act synergistically.

A86. The method of any one of Embodiments A1-A85, wherein the method further comprises administering cetuximab to the patient.

A87. The method of any one of Embodiments A1-A86, wherein the method improves the efficacy of cetuximab.

A88. The method of any one of Embodiments A1-A87, wherein the PI3K inhibitor is selected from the group comprising alpelisib (BYL719), AMG319, AZD8168, AZD8835, buparlisib, B591, CH5132799, copanlisib (aliqopa), delalisib (zydelig), duvelisib (copiktra), eganelisib, GSK2636771, leniolisib, linperlisib, parsaclisib, pictilisib, pilaralisib, RIDR-PI-103, serabelisib, sonolisib, taselisib, tenalisib, TG-100-115, umbralisib, zandelisib, or ZSTK474, or a pharmaceutically acceptable form thereof.

A89. The method of any one of Embodiments A1-A88, wherein the PI3K inhibitor is alpelisib.

Embodiments A90-A94 intentionally left blank

A95. The method of any one of Embodiments A1-A94, wherein the method increases Time To Progression (TTP), Progression Free Survival (PFS), Event-free survival (EFS), Overall Survival (OS), overall response rate (ORR), or duration of response (DoR), disease control rate (DCR; complete response (CR) plus partial response (PR) plus stable disease (SD)), rate of CR, or rate of SD, or combinations thereof, compared to single agent therapy, first-line therapy, second-line therapy, anti-EGFR antibody therapy, immunotherapy, or localized and loco-regional disease therapy.

A96. The method of any one of Embodiments A1-A95, wherein the method increases TPP compared to single agent therapy, first-line therapy, second-line therapy, anti-EGFR antibody therapy, immunotherapy, or localized and loco-regional disease therapy.

A97. The method of any one of Embodiments A1-A96, wherein the method increases PFS compared to single agent therapy, first-line therapy, second-line therapy, anti-EGFR antibody therapy, immunotherapy, or localized and loco-regional disease therapy.

A98. The method of any one of Embodiments A1-A97, wherein the method increases EFS compared to single agent therapy, first-line therapy, second-line therapy, anti-EGFR antibody therapy, immunotherapy, or localized and loco-regional disease therapy.

A99. The method of any one of Embodiments A1-A98, wherein the method increases OS compared to single agent therapy, first-line therapy, second-line therapy, anti-EGFR antibody therapy, immunotherapy, or localized and loco-regional disease therapy.

A100. The method of any one of Embodiments A1-A99, wherein the method increases ORR compared to single agent therapy, first-line therapy, second-line therapy, anti-EGFR antibody therapy, immunotherapy, or localized and loco-regional disease therapy.

A101. The method of any one of Embodiments A1-A100, wherein the method increases DoR compared to single agent therapy, first-line therapy, second-line therapy, anti-EGFR antibody therapy, immunotherapy, or localized and loco-regional disease therapy.

A102. The method of any one of Embodiments A1-A101, wherein the method decreases time to response (TTR), compared to single agent therapy, first-line therapy, second-line therapy, anti-EGFR antibody therapy, immunotherapy, or localized and loco-regional disease therapy.’

A103. A method of treating head and neck squamous cell carcinoma (HNSCC) in a patient, comprising administering to the patient (a) tipifarnib and (b) alpelisib, wherein the tipifarnib and the alpelisib are administered sequentially or consecutively, and wherein the tipifarnib is administered once or twice per day, preferably twice per day, on days 1-7 and 15-21 of a 28-day cycle and the alpelisib is administered once per day every day of the 28-day cycle.

A104. A method of treating head and neck squamous cell carcinoma (HNSCC) in a patient, comprising administering to the patient (a) tipifarnib and (b) alpelisib, wherein the tipifarnib and the alpelisib are administered sequentially or consecutively, and wherein the tipifarnib is administered once or twice per day, preferably twice per day, on days 1-7 of a 28-day cycle and the alpelisib is administered once per day every day of the 28-day cycle.

A105. A method of treating head and neck squamous cell carcinoma (HNSCC) in a patient, comprising administering to the patient (a) tipifarnib and (b) alpelisib, wherein the tipifarnib and the alpelisib are administered sequentially or consecutively, and wherein the tipifarnib is administered once or twice per day, preferably twice per day, on days 1-21 of a 28-day cycle and the alpelisib is administered once per day every day of the 28-day cycle.

A106. A method of treating head and neck squamous cell carcinoma (HNSCC) in a patient, comprising administering to the patient (a) tipifarnib and (b) alpelisib, wherein the tipifarnib and the alpelisib are administered sequentially or consecutively, and wherein the tipifarnib is administered once or twice per day, preferably twice per day, every day of a 28-day cycle and the alpelisib is administered once per day every day of the 28-day cycle.

A107. A method of treating head and neck squamous cell carcinoma (HNSCC) in a patient, comprising administering to the patient (a) tipifarnib and (b) alpelisib, wherein the tipifarnib and the alpelisib are administered sequentially or consecutively in a 28-day cycle, and wherein at least one day of the 28-day cycle the tipifarnib and the alpelisib are each administered to the patient.

A108. The method of Embodiment A107, wherein the tipifarnib and the alpelisib are each administered to the patient for 2, 3, 4, 5, 6, or 7 days of the 28-day cycle.

A109. The method of Embodiment A107, wherein the tipifarnib and the alpelisib are each administered to the patient for 14 days of the 28-day cycle.

A110. The method of Embodiment A107, wherein the tipifarnib and the alpelisib are each administered to the patient for 21 days of the 28-day cycle.

A111. The method of Embodiment A107, wherein the tipifarnib and the alpelisib are each administered to the patient for 28 days of the 28-day cycle.

A112. The method of any one of Embodiments A103-A110, wherein the method is further defined as detailed in any one of Embodiments A4-A102.

A113. The method of any one of Embodiments A1-A112, wherein 50% or more of cells in an obtained HNSCC tissue sample from the patient have a Cytoplasmic Percent Score of ≥3+, as determined by the HRAS immunohistochemistry (IHC) assay.

A114. The method of any one of Embodiments A1-A113, wherein 50% or more of cells in an obtained HNSCC tissue sample from the patient have a Plasma Membrane Percent Score of ≥3+, as determined by the HRAS immunohistochemistry (IHC) assay.

A115. The method of any one of Embodiments A1-A114, wherein the HNSCC has an overexpression of wild-type HRas protein, as determined by an HRAS immunohistochemistry (IHC) assay.

B1. A pharmaceutical composition, comprising (a) tipifarnib, (b) a PI3K inhibitor, or a pharmaceutically acceptable salt thereof, preferably alpelisib, such as 10-400 mg of alpelisib, and (c) a pharmaceutically acceptable carrier, excipient or diluent.

B2. The pharmaceutical composition of Embodiment B1, wherein the pharmaceutical composition comprises a therapeutically effective amount of the tipifarnib.

B3. The pharmaceutical composition of Embodiment B1 or Embodiment B2, wherein the pharmaceutical composition of comprises 1-1000 mg of the tipifarnib.

B4. The pharmaceutical composition of any one of Embodiments B1-B3, wherein the pharmaceutical composition comprises 1-5 mg, 1-10 mg, 1-25 mg, 1-50 mg, 1-75 mg, 1-100 mg, 1-300 mg, 1-600 mg, 1-900 mg, 50-75 mg, 50-100 mg, 50-150 mg, 50-200 mg, 50-250 mg, 50-300 mg, 50-600 mg, 50-900 mg, 100-300 mg, 200-400 mg, 300-600 mg, 300-900 mg, 400-600 mg, 500-700 mg, 600-900 mg, and 800-1000 mg of the tipifarnib.

B5. The pharmaceutical composition of any one of Embodiments B1-B4, wherein the pharmaceutical composition comprises about 100 mg, about 200 mg, about 300 mg, about 400 mg, about 600 mg, about 900 mg, or about 1200 mg of the tipifarnib.

B6. The pharmaceutical composition of any one of Embodiments B1-B5, wherein the pharmaceutical composition comprises about 300 mg of the tipifarnib.

B7. The pharmaceutical composition of any one of Embodiments B1-B5, wherein the pharmaceutical composition comprises about 600 mg of the tipifarnib.

B8. The pharmaceutical composition of any one of Embodiments B1-B5, wherein the pharmaceutical composition comprises about 900 mg of the tipifarnib.

B9. The pharmaceutical composition of any one of Embodiments B1-B8, wherein the pharmaceutical composition comprises a therapeutically effective amount of the PI3K inhibitor, or pharmaceutically acceptable salt thereof, preferably alpelisib.

B10. The pharmaceutical composition of any one of Embodiments B1-B9, wherein the PI3K inhibitor is selected from the group comprising alpelisib (BYL719), AMG319, AZD8168, AZD8835, buparlisib, B591, CH5132799, copanlisib (aliqopa), delalisib (zydelig), duvelisib (copiktra), eganelisib, GSK2636771, leniolisib, linperlisib, parsaclisib, pictilisib, pilaralisib, RIDR-PI-103, serabelisib, sonolisib, taselisib, tenalisib, TG-100-115, umbralisib, zandelisib, or ZSTK474, or a pharmaceutically acceptable form thereof.

B11. The pharmaceutical composition of any one of Embodiments B1-B10, wherein the PI3K inhibitor is alpelisib.

B12. The pharmaceutical composition of Embodiment B11, wherein the pharmaceutical composition comprises 10-400 mg of the alpelisib.

B13. The pharmaceutical composition of Embodiment B11 or Embodiment B12, wherein the pharmaceutical composition comprises 10-300 mg, 10-200 mg, 10-150 mg, 10-100 mg, 10-50 mg, 25-400 mg, 25-300 mg, 25-200 mg, 25-150 mg, 25-100 mg, 25-50 mg, 50-400 mg, 50-300 mg, 50-200 mg, 50-150 mg, 50-100 mg, 100-400 mg, 100-300 mg, 100-200 mg, 150-250 mg, 175-225 mg, 200-400 mg, or 200-300 mg of the alpelisib.

B14. The pharmaceutical composition of any one of Embodiments B11-B13, wherein the pharmaceutical composition comprises about 25 mg, about 50 mg, about 100 mg, about 150 mg, about 200 mg, about 250 mg, or about 300 mg of alpelisib.

B15. The pharmaceutical composition of any one of Embodiments B11-B14, wherein the pharmaceutical composition comprises about 50 mg, about 150 mg, or about 200 mg of the alpelisib.

B16. The pharmaceutical composition of any one of Embodiments B11-B15, wherein the pharmaceutical composition comprises about 50 mg of the alpelisib.

B17. The pharmaceutical composition of any one of Embodiments B11-B15, wherein the pharmaceutical composition comprises about 150 mg of the alpelisib.

B18. The pharmaceutical composition of any one of Embodiments B11-B15, wherein the pharmaceutical composition comprises about 200 mg of the alpelisib.

B19. A pharmaceutical kit comprising the pharmaceutical composition of any one of Embodiments B1-B18.

B20. A pharmaceutical packaging comprising the pharmaceutical composition of any one of Embodiments B1-B18.

B21. A pharmaceutical kit, comprising (a) a pharmaceutical composition comprising tipifarnib and a pharmaceutically acceptable carrier, excipient or diluent, and (b) a pharmaceutical composition comprising a PI3K inhibitor, or a pharmaceutically acceptable salt thereof, preferably alpelisib, such as 10-400 mg of alpelisib, and a pharmaceutically acceptable carrier, excipient or diluent.

B22. A pharmaceutical packaging, comprising (a) a pharmaceutical composition comprising tipifarnib and a pharmaceutically acceptable carrier, excipient or diluent, and (b) a pharmaceutical composition comprising a PI3K inhibitor, or a pharmaceutically acceptable salt thereof, preferably alpelisib, such as 10-400 mg of alpelisib, and a pharmaceutically acceptable carrier, excipient or diluent.

B23. The pharmaceutical kit or pharmaceutical packaging of any one of Embodiments B19-B22, wherein the pharmaceutical kit or pharmaceutical packaging further comprises instructions for administering the contents of the packaging to a subject having HNSCC.

B24. The pharmaceutical kit or pharmaceutical packaging of any one of Embodiments B21-B23, wherein the pharmaceutical composition of (a) comprises a therapeutically effective amount of the tipifarnib.

B25. The pharmaceutical kit or pharmaceutical packaging of any one of Embodiments B21-B24, wherein the pharmaceutical composition of (a) comprises 1-1000 mg of the tipifarnib.

B26. The pharmaceutical kit or pharmaceutical packaging of any one of Embodiments B21-B25, wherein the pharmaceutical composition of (a) comprises 1-5 mg, 1-10 mg, 1-25 mg, 1-50 mg, 1-75 mg, 1-100 mg, 1-300 mg, 1-600 mg, 1-900 mg, 50-75 mg, 50-100 mg, 50-150 mg, 50-200 mg, 50-250 mg, 50-300 mg, 50-600 mg, 50-900 mg, 100-300 mg, 200-400 mg, 300-600 mg, 300-900 mg, 400-600 mg, 500-700 mg, 600-900 mg, and 800-1000 mg of the tipifarnib.

B27. The pharmaceutical kit or pharmaceutical packaging of any one of Embodiments B21-B26, wherein the pharmaceutical composition of (a) comprises about 100 mg, about 200 mg, about 300 mg, about 400 mg, about 600 mg, about 900 mg, or about 1200 mg of the tipifarnib.

B28. The pharmaceutical kit or pharmaceutical packaging of any one of Embodiments B21-B27, wherein the pharmaceutical composition of (a) comprises about 300 mg of the tipifarnib.

B29. The pharmaceutical kit or pharmaceutical packaging of any one of Embodiments B21-B27, wherein the pharmaceutical composition of (a) comprises about 600 mg of the tipifarnib.

B30. The pharmaceutical kit or pharmaceutical packaging of any one of Embodiments B21-B27, wherein the pharmaceutical composition of (a) comprises about 900 mg of the tipifarnib.

B31. The pharmaceutical kit or pharmaceutical packaging of any one of Embodiments B21-B30, wherein the pharmaceutical composition of (b) comprises a therapeutically effective amount of the PI3K inhibitor, or pharmaceutically acceptable salt thereof, preferably alpelisib.

B32. The pharmaceutical kit or pharmaceutical packaging of any one of Embodiments B21-B31, wherein the PI3K inhibitor is selected from the group comprising alpelisib (BYL719), AMG319, AZD8168, AZD8835, buparlisib, B591, CH5132799, copanlisib (aliqopa), delalisib (zydelig), duvelisib (copiktra), eganelisib, GSK2636771, leniolisib, linperlisib, parsaclisib, pictilisib, pilaralisib, RIDR-PI-103, serabelisib, sonolisib, taselisib, tenalisib, TG-100-115, umbralisib, zandelisib, or ZSTK474, or a pharmaceutically acceptable form thereof.

B33. The pharmaceutical kit or pharmaceutical packaging of any one of Embodiments B21-B32, wherein the PI3K inhibitor is alpelisib.

B34. The pharmaceutical kit or pharmaceutical packaging of Embodiment B33, wherein the pharmaceutical composition of (b) comprises 10-400 mg of the alpelisib.

B35. The pharmaceutical kit or pharmaceutical packaging of Embodiment B33 or Embodiment B34, wherein the pharmaceutical composition of (b) comprises 10-300 mg, 10-200 mg, 10-150 mg, 10-100 mg, 10-50 mg, 25-400 mg, 25-300 mg, 25-200 mg, 25-150 mg, 25-100 mg, 25-50 mg, 50-400 mg, 50-300 mg, 50-200 mg, 50-150 mg, 50-100 mg, 100-400 mg, 100-300 mg, 100-200 mg, 150-250 mg, 175-225 mg, 200-400 mg, or 200-300 mg of the alpelisib.

B36. The pharmaceutical kit or pharmaceutical packaging of any one of Embodiments B33-B35, wherein the pharmaceutical composition of (b) comprises about 25 mg, about 50 mg, about 100 mg, about 150 mg, about 200 mg, about 250 mg, or about 300 mg of alpelisib.

B37. The pharmaceutical kit or pharmaceutical packaging of any one of Embodiments B33-B36, wherein the pharmaceutical composition of (b) comprises about 50 mg, about 150 mg, or about 200 mg of the alpelisib.

B38. The pharmaceutical kit or pharmaceutical packaging of any one of Embodiments B33-B36, wherein the pharmaceutical composition of (b) comprises 50 mg of the alpelisib.

B39. The pharmaceutical kit or pharmaceutical packaging of any one of Embodiments B33-B36, wherein the pharmaceutical composition of (b) comprises 150 mg of the alpelisib.

B40. The pharmaceutical kit or pharmaceutical packaging of any one of Embodiments B33-B36, wherein the pharmaceutical composition of (b) comprises 200 mg of the alpelisib.

B41. A pharmaceutical kit, comprising (a) tipifarnib and (b) a PI3K inhibitor, preferably alpelisib, such as 10-400 mg of alpelisib.

B42. A pharmaceutical packaging, comprising (a) tipifarnib and (b) a PI3K inhibitor, preferably alpelisib, such as 10-400 mg of alpelisib.

B43. The pharmaceutical kit or pharmaceutical packaging of Embodiment B41 or Embodiment B42, wherein the pharmaceutical kit or pharmaceutical packaging further comprises instructions for administering the contents of the packaging to a subject having HNSCC.

B44. The pharmaceutical kit or pharmaceutical packaging of any one of Embodiments B41-B43, wherein the pharmaceutical kit or pharmaceutical packaging comprises a therapeutically effective amount of the tipifarnib.

B45. The pharmaceutical kit or pharmaceutical packaging of any one of Embodiments B41-B44, wherein the pharmaceutical kit or pharmaceutical packaging comprises 1-1000 mg of the tipifarnib.

B46. The pharmaceutical kit or pharmaceutical packaging of any one of Embodiments B41-B45, wherein the pharmaceutical kit or pharmaceutical packaging comprises 1-5 mg, 1-10 mg, 1-25 mg, 1-50 mg, 1-75 mg, 1-100 mg, 1-300 mg, 1-600 mg, 1-900 mg, 50-75 mg, 50-100 mg, 50-150 mg, 50-200 mg, 50-250 mg, 50-300 mg, 50-600 mg, 50-900 mg, 100-300 mg, 200-400 mg, 300-600 mg, 300-900 mg, 400-600 mg, 500-700 mg, 600-900 mg, and 800-1000 mg of the tipifarnib.

B47. The pharmaceutical kit or pharmaceutical packaging of any one of Embodiments B41-B46, wherein the pharmaceutical kit or pharmaceutical packaging comprises about 100 mg, about 200 mg, about 300 mg, about 400 mg, about 600 mg, about 900 mg, or about 1200 mg of the tipifarnib.

B48. The pharmaceutical kit or pharmaceutical packaging of any one of Embodiments B41-B47, wherein the pharmaceutical kit or pharmaceutical packaging comprises about 300 mg of the tipifarnib.

B49. The pharmaceutical kit or pharmaceutical packaging of any one of Embodiments B41-B47, wherein the pharmaceutical kit or pharmaceutical packaging comprises about 600 mg of the tipifarnib.

B50. The pharmaceutical kit or pharmaceutical packaging of any one of Embodiments B41-B47, wherein the pharmaceutical kit or pharmaceutical packaging comprises about 900 mg of the tipifarnib.

B51. The pharmaceutical kit or pharmaceutical packaging of any one of Embodiments B41-B50, wherein the pharmaceutical kit or pharmaceutical packaging comprises a therapeutically effective amount of the PI3K inhibitor, or pharmaceutically acceptable salt thereof, preferably alpelisib.

B52. The pharmaceutical kit or pharmaceutical packaging of any one of Embodiments B41-B51, wherein the PI3K inhibitor is selected from the group comprising alpelisib (BYL719), AMG319, AZD8168, AZD8835, buparlisib, B591, CH5132799, copanlisib (aliqopa), delalisib (zydelig), duvelisib (copiktra), eganelisib, GSK2636771, leniolisib, linperlisib, parsaclisib, pictilisib, pilaralisib, RIDR-PI-103, serabelisib, sonolisib, taselisib, tenalisib, TG-100-115, umbralisib, zandelisib, or ZSTK474, or a pharmaceutically acceptable form thereof.

B53. The pharmaceutical kit or pharmaceutical packaging of any one of Embodiments B41-B52, wherein the PI3K inhibitor is alpelisib.

B54. The pharmaceutical kit or pharmaceutical packaging of Embodiment B53, wherein the pharmaceutical kit or pharmaceutical packaging comprises 10-400 mg of the alpelisib.

B55. The pharmaceutical kit or pharmaceutical packaging of Embodiment B53 or Embodiment B54, wherein the pharmaceutical kit or pharmaceutical packaging comprises 10-300 mg, 10-200 mg, 10-150 mg, 10-100 mg, 10-50 mg, 25-400 mg, 25-300 mg, 25-200 mg, 25-150 mg, 25-100 mg, 25-50 mg, 50-400 mg, 50-300 mg, 50-200 mg, 50-150 mg, 50-100 mg, 100-400 mg, 100-300 mg, 100-200 mg, 150-250 mg, 175-225 mg, 200-400 mg, or 200-300 mg of the alpelisib.

B56. The pharmaceutical kit or pharmaceutical packaging of any one of Embodiments B53-B55, wherein the pharmaceutical kit or pharmaceutical packaging comprises about 25 mg, about 50 mg, about 100 mg, about 150 mg, about 200 mg, about 250 mg, or about 300 mg of alpelisib.

B57. The pharmaceutical kit or pharmaceutical packaging of any one of Embodiments B53-B56, wherein the pharmaceutical kit or pharmaceutical packaging comprises about 50 mg, about 150 mg, or about 200 mg of the alpelisib.

B58. The pharmaceutical kit or pharmaceutical packaging of any one of Embodiments B53-B56, wherein the pharmaceutical kit or pharmaceutical packaging comprises 50 mg of the alpelisib.

B59. The pharmaceutical kit or pharmaceutical packaging of any one of Embodiments B53-B56, wherein the pharmaceutical kit or pharmaceutical packaging comprises 150 mg of the alpelisib.

B60. The pharmaceutical kit or pharmaceutical packaging of any one of Embodiments B53-B56, wherein the pharmaceutical kit or pharmaceutical packaging comprises 200 mg of the alpelisib.

B61. The method of any one of Embodiments A1-A102, wherein the method administers the pharmaceutical composition, pharmaceutical kit, or pharmaceutical packaging of any one of Embodiments B1-B60.

C1. An HRAS immunohistochemistry (IHC) assay, comprising:
a) obtaining a tumor tissue sample from a patient having or suspected of having a head and neck squamous cell carcinoma (HNSCC);
b) preparing a slide from a section of the tumor tissue sample;
c) dewaxing and unmasking antigens from the slide;
d) immunohistochemically treating the dewaxed and antigen unmasked slide with a monoclonal antibody to human HRAS;
e) transferring the IHC treated slides to a cover slip area;
f) dehydrating the transferred slides; and
g) detecting intensity and cellular location of a visually detectable signal of HRAS protein expression from the dehydrated slide.

C2. A method of immunohistochemically staining a sample for HRAS protein expression, comprising:
a) obtaining a tumor tissue sample from a human subject having or suspected of having a head and neck squamous cell carcinoma (HNSCC);
b) fixing the tumor tissue sample in formalin;
c) embedding the formalin-fixed tumor tissue sample in a paraffin block;
d) slicing the paraffin block into tissue sections having a thickness of about 4 μm to about 5 μm and mounting the sections on glass slides;
e) dewaxing tissue sections on the glass slides;
f) contacting the dewaxed tissue sections with a monoclonal antibody to human HRAS;
g) contacting the antibody-contacted tissue section with a reagent that detects the monoclonal antibody to human HRAS; and
h) incubating the tissue section in a chromogen solution containing 3,3′-diaminobenzidine to react with the reagent that detects the monoclonal antibody to human HRAS to generate a visually detectable signal,
   wherein the visually detectable signal is a stain for the cellular location of HRAS protein expression in the tumor tissue.

C3. The method of Embodiment C2, wherein the method further comprises i) transferring the incubated tissue section to a cover slip area.

C4. The method of Embodiment C3, wherein the method further comprises j) dehydrating the transferred tissue section.

C5. The method of any one of Embodiments C2-C4, wherein the method further comprises k) detecting visually detectable signal intensity and cellular location of HRAS protein expression.

C6. The assay of Embodiment C1 or the method of Embodiments C2-C5, wherein the assay or the method optionally comprises staining the tumor tissue sample with hematoxylin and/or eosin.

C7. The assay of Embodiment C1 or the method of Embodiments C2-C6, wherein the monoclonal antibody to human HRAS is ARC0098.

C8. The assay of Embodiment C1 or the method of Embodiments C2-C7, wherein the assay or the method is performed on an automated platform.

C9. A method for identifying the level of HRAS expression in a head and neck squamous cell carcinoma (HNSCC) patient or for identifying a HNSCC patient who is likely to respond to a combination therapy comprising:
a) obtaining a tissue sample from a subject having or suspected of having HNSCC;
b) immunohistochemically staining the tissue sample for HRAS protein expression;
c) calculating a percent score based on intensity and cellular location of the HRAS protein expression in the immunohistochemically stained tissue sample; and
d) (for the method of identifying a HNSCC patient who is likely to respond to a combination therapy, and optionally for the method for identifying the level of HRAS expression in a HNSCC patient) determining the likely responsiveness of the HNSCC patient to the combination therapy based on the percent score, wherein the combination therapy comprises a farnesyltransferase inhibitor and a PI3K inhibitor.

C10. The method of Embodiment C9, wherein the percent score is calculated separately for cytoplasmic expression and membrane expression of the HRAS protein in the cells of the immunohistochemically stained tissue sample.

C11. The method of Embodiment C9 or Embodiment C10, wherein the percent score is calculated by summing the percentages of HRAS staining intensity, wherein HRAS staining intensity is determined on a four-point semi-quantitative scale where 0=null, negative or non-specific staining, 1+=low or weak staining, 2+=medium or moderate staining, and 3+=high or strong staining.

C12. The method of any one of Embodiments C9-C11, wherein the HNSCC patient is likely to respond to the combination therapy when 50% or more of the cells in the immunohistochemically stained tissue sample have a cytoplasmic percent score of ≥3+ or when 50% or more of the cells in the immunohistochemically stained tissue sample have a plasma membrane percent score of ≥3+.

C13. The method of any one of Embodiments C9-C12, wherein the HNSCC is an HRAS overexpressing HNSCC when 50% or more of the cells in the immunohistochemically stained tissue sample have a cytoplasmic percent score of ≥3+ or, preferably, when 50% or more of the cells in the immunohistochemically stained tissue sample have a plasma membrane percent score of ≥3+.

C14. The method of any one of Embodiments C9-C13, wherein 50% or more of the cells in the immunohistochemically stained tissue sample have a cytoplasmic percent score of ≥3+.

C15. The method of any one of Embodiments C9-C14, wherein 50% or more of the cells in the immunohistochemically stained tissue sample have a plasma membrane percent score of ≥3+.

C16. The method of any one of Embodiments C9-C15, wherein the HNSCC patient tissue sample has an overexpression of HRAS protein.

C17. The method of any one of Embodiments C9-C16, wherein the HNSCC patient has an HRAS overexpressing HNSCC.

C18. The method of any one of Embodiments C9-C17, wherein the HRAS is wild type HRAS.

C19. The method of any one of Embodiments C9-C18, wherein the farnesyltransferase inhibitor is tipifarnib.

C20. The method of any one of Embodiments C9-C19, wherein the PI3K inhibitor is alpelisib.

D1. A kit for use in a HRAS immunohistochemistry (IHC) assay, comprising a monoclonal antibody for detection of human HRAS expression and at least one reagent for carrying out the HRAS IHC assay.

D2. The kit of Embodiment D1, wherein the kit further comprises a secondary antibody that reacts with the monoclonal antibody for detection of human HRAS protein expression.

D3. The kit of Embodiment D1 or Embodiment D2, wherein the kit further comprises a reagent for visualizing the monoclonal antibody for detection of human HRAS protein expression.

D4. The kit of any one of Embodiments D1-D3, wherein the kit further comprises a package insert and/or instructions to carry out the HRAS IHC assay.

D5. The kit of any one of Embodiments D1-D4, wherein the kit further comprises a package insert and/or instructions to score the results of the HRAS IHC assay.

The embodiments described above are intended to be merely exemplary, and those skilled in the art will recognize, or are able to ascertain using no more than routine experimentation, numerous equivalents of specific compounds, materials, and procedures. All such equivalents are considered to be within the scope of the invention and are encompassed by the appended claims.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference in its entirety. In case of conflict, the present application, including any definitions herein, will control.

1. A method of treating head and neck squamous cell carcinoma (HNSCC) in a patient, comprising administering to the patient (a) tipifarnib and (b) 10-400 mg of alpelisib.

2. A method of mitigating, slowing the progression of, or overcoming cetuximab-resistance in a patient having HNSCC, comprising administering to the HNSCC patient (a) tipifarnib and (b) alpelisib; wherein the HNSCC patient is currently being treated, or was previously treated, with cetuximab.

3. The method of claim 1, wherein the method delays, halts or prevents progression of HNSCC.

4. The method of claim 1, wherein the method mitigates or reduces the severity of at least one symptom associated with HNSCC.

5. The method of claim 1, wherein the method mitigates, slows the progression of, or overcomes cetuximab-resistance.

6. (canceled)

7. (canceled)

8. The method of claim 1, wherein the HNSCC has an overexpression of wild-type HRas protein.

9. The method of claim 1, wherein the HNSCC has an HRas protein mutation.

10. The method of claim 1, wherein the HNSCC is an HRAS-mutant dependent HNSCC.

11. (canceled)

12. The method of claim 1, wherein the HNSCC is a PIK3CA-dependent HNSCC.

13. The method of claim 1, wherein the HNSCC has a PIK3CA alteration or dysregulation.

14. The method of claim 13, wherein the PIK3CA alteration or dysregulation is a PIK3CA mutation.

15. (canceled)

16. (canceled)

17. The method of claim 13, wherein the PIK3CA alteration or dysregulation is a PIK3CA gene amplification.

18. The method of claim 13, wherein the PIK3CA alteration or dysregulation is a PIK3CA copy gain.

19. The method of claim 1, wherein the HNSCC is early stage, advanced, relapsed, refractory, or metastatic HNSCC.

20. The method of claim 1, wherein the HNSCC is recurrent/metastatic HNSCC.

21. The method of claim 1, wherein the tipifarnib and the alpelisib are administered concurrently or sequentially.

22. (canceled)

23. (canceled)

24. The method of claim 1, wherein the tipifarnib is administered to the patient orally.

25. (canceled)

26. (canceled)

27. The method of claim 1, wherein the tipifarnib is administered at a dose of about 100 mg, about 200 mg, about 300 mg, about 400 mg, about 600 mg, about 900 mg, or about 1200 mg per day, or about 1800 mg per day.

28. The method of claim 1, wherein the tipifarnib is administered 1, 2, 3, or 4 times per day.

29. (canceled)

30. (canceled)

31. The method of claim 1, wherein the alpelisib is administered to the patient orally.

32. The method of claim 1, wherein the alpelisib is administered to the patient at a dose of about 50 mg, about 100 mg, about 150 mg, about 200 mg, about 250 mg or about 300 mg per day.

33. The method of claim 1, wherein the alpelisib is administered to the patient once or twice per day.

34. (canceled)

35. The method of claim 1, wherein the tipifarnib is administered once or twice per day, on days 1-7 of a 28-day cycle and the alpelisib is administered once per day of a 28-day cycle.

36. The method of claim 1, wherein the tipifarnib is administered once or twice per day on days 1-7 and 15-21 of a 28-day cycle and the alpelisib is administered once per day of a 28-day cycle.

37. The method of claim 1, wherein the tipifarnib is administered once or twice per day on days 1-21 of a 28-day cycle and the alpelisib is administered once per day of a 28-day cycle.

38. The method of claim 1, wherein the tipifarnib and the alpelisib are administered once or twice per day of a 28-day cycle.

39. The method of claim 1, wherein the tipifarnib and the alpelisib are administered for one or more 28-day cycles.

40. (canceled)

41. (canceled)

42. The method of claim 1, wherein the method increases Time To Progression (TTP), Progression Free Survival (PFS), Event-free survival (EFS), Overall Survival (OS), overall response rate (ORR), or duration of response (DoR), or combinations thereof, compared to single agent therapy, first-line therapy, second-line therapy, anti-EGFR antibody therapy, immunotherapy, or localized and loco-regional disease therapy.

43. The method of claim 1, wherein the method decreases time to response (TTR), compared to single agent therapy, first-line therapy, second-line therapy, anti-EGFR antibody therapy, immunotherapy, or localized and loco-regional disease therapy.

44. A pharmaceutical kit, comprising (a) a pharmaceutical composition comprising tipifarnib, and a pharmaceutically acceptable carrier, excipient or diluent, and (b) a pharmaceutical composition comprising 10-400 mg of alpelisib and a pharmaceutically acceptable carrier, excipient or diluent.

45. (canceled)
